SEQUENCE LISTING (59 sequences)

SEQ ID NO: 1 - protein, 414 aa, Homo sapiens
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 2-9 - skipped (8 entries)

SEQ ID NO: 10 - protein, 120 aa, synthetic construct
EVQLVESGGDLVKPGGSLKLSCAASGFTFANYAMSWIRQTPEKRLEWVATISGGGLYINYPANLKGRFTISRDNAKNNLYLQMSHLKSEDTAMYYCVKDRGQNWSWFAYWGQGTLVTVSA

SEQ ID NO: 11 - protein, 5 aa, synthetic construct
NYAMS

SEQ ID NO: 12 - protein, 17 aa, synthetic construct
TISGGGLYINYPANLKG

SEQ ID NO: 13 - protein, 11 aa, synthetic construct
DRGQNWSWFAY

SEQ ID NO: 14 - protein, 115 aa, synthetic construct
QLVLTQSSSASFSLGASAKLTCTLSSQHSTYTIEWYQQQPLKPPKYVMELEKDGSHSTGDGIPDRFSGSSSGADRYLSISNIQPEDEAIYICGVGDTIKEQFVYVFGGGTKVTVL

SEQ ID NO: 15 - protein, 12 aa, synthetic construct
TLSSQHSTYTIE

SEQ ID NO: 16 - protein, 11 aa, synthetic construct
LEKDGSHSTGD

SEQ ID NO: 17 - protein, 13 aa, synthetic construct
GVGDTIKEQFVYV

SEQ ID NO: 18 - DNA, 360 bp, synthetic construct
  source 1..360: mol_type = other DNA
gaagtgcaactggtggagtctgggggagacttagtgaagcctggagggtccctgaaactctcctgtgcagcctctggattcactttcgctaactatgccatgtcttggattcgccagactccggaaaagaggctggagtgggtcgcaaccatttctggtggtggtctttatatcaattatccagccaatttaaagggccgattcaccatctccagagacaatgccaagaacaacttgtacctgcaaatgagccatctgaagtctgaagacacagccatgtattactgtgttaaagataggggccaaaactggtcctggtttgcctactggggccaagggactctggtcactgtctctgca

SEQ ID NO: 19 - DNA, 345 bp, synthetic construct
  source 1..345: mol_type = other DNA
caacttgtgctcactcagtcatcttcagcctctttctccctgggagcctcagcaaaactcacgtgcaccttgagtagtcagcacagtacgtacaccattgaatggtatcagcaacagccactcaagcctcctaagtatgtgatggaacttgagaaagatggaagccacagcacaggtgatgggattcctgatcgcttttctggatccagctctggtgctgatcgctaccttagcatttccaacatccagcctgaagatgaagcaatatacatctgtggtgtgggtgatacaattaaggaacaatttgtgtatgttttcggcggtggaaccaaggtcactgtccta

SEQ ID NO: 20 - protein, 120 aa, synthetic construct
EVQLVESGGVLVKPGGSLKLSCAASGFIFSSFSMSWVRQNPEKRLEWVATISAAGRYIYYPDNVKGRFTISRDNAKNNLYLYMSHVRSEDTAIYYCVKDRGLNWSWFAYWGQGTLVTVSA

SEQ ID NO: 21 - protein, 5 aa, synthetic construct
SFSMS

SEQ ID NO: 22 - protein, 17 aa, synthetic construct
TISAAGRYIYYPDNVKG

SEQ ID NO: 23 - protein, 11 aa, synthetic construct
DRGLNWSWFAY

SEQ ID NO: 24 - protein, 115 aa, synthetic construct
QLVLTQSSSASFSLGASAKLTCTLSSQHSTYTIEWYQQQPLKPPKYVMELKKDGSHSTGDGIPDRFSGSSSGADRYLSISDIQPEDEAVYICGVGDTIKEQFVYVFGGGTKVTVL

SEQ ID NO: 25 - skipped

SEQ ID NO: 26 - protein, 11 aa, synthetic construct
LKKDGSHSTGD

SEQ ID NO: 27 - skipped

SEQ ID NO: 28 - DNA, 360 bp, synthetic construct
  source 1..360: mol_type = other DNA
gaagtgcagctggtggagtctggtggagtcttagtgaagcctggagggtccctgaaactctcctgtgcagcctctggattcattttcagtagcttttccatgtcttgggttcgccagaatccggaaaagaggctggagtgggtcgcaaccattagtgctgctggtcgttacatttactatccagacaatgttaagggccgattcaccatctccagagacaatgccaagaacaacctgtacctgtacatgagccatgtgaggtctgaggacacagccatctattactgtgtaaaagataggggcctaaactggtcctggtttgcttactggggccaagggactctggtcactgtctctgca

SEQ ID NO: 29 - DNA, 345 bp, synthetic construct
  source 1..345: mol_type = other DNA
caacttgtgctcactcagtcatcttcagcctctttctccctgggagcctcagcaaaactcacgtgcaccttgagtagtcagcacagtacgtacaccattgaatggtatcaacaacagccactcaagcctcctaagtatgtgatggagcttaagaaagatggaagccacagcacaggtgatgggattcctgatcgcttctctggatccagctctggtgctgatcgctaccttagcatttccgacatccagcctgaagatgaagcagtatacatctgtggtgtgggtgatacaattaaggaacaatttgtgtatgttttcggcggtggaaccaaggtcactgtccta

SEQ ID NO: 30 - protein, 111 aa, synthetic construct
EVQLQQSGPELVKPGASVKISCKTSGYTFTEYTMHWVKQSHGQSLEWIGGIHPNNGGTIYNQKFKGRATLTVDKSSSTAYMEFRSLTSDDSAVYYCASPCWGQGTSVTVSS

SEQ ID NO: 31 - protein, 5 aa, synthetic construct
EYTMH

SEQ ID NO: 32 - protein, 17 aa, synthetic construct
GIHPNNGGTIYNQKFKG

SEQ ID NO: 33 - skipped

SEQ ID NO: 34 - protein, 112 aa, synthetic construct
DVVMTQTPLTLSVSIGQPASISCKSSQSLLHDDGKTYLNWLLQRPGQSPKRLISLVSKLDSKFPDRFTGSGSGTDFTLKISRVEAEDLGVYYCWQGTHFPHTFGAGTKLELK

SEQ ID NO: 35 - protein, 16 aa, synthetic construct
KSSQSLLHDDGKTYLN

SEQ ID NO: 36 - protein, 7 aa, synthetic construct
LVSKLDS

SEQ ID NO: 37 - protein, 9 aa, synthetic construct
WQGTHFPHT

SEQ ID NO: 38 - DNA, 333 bp, synthetic construct
  source 1..333: mol_type = other DNA
gaggtccagctacaacagtctggacctgagctggtgaagcctggggcttcagtgaagatatcctgcaagacttctggatacacattcactgaatacaccatgcactgggtgaagcagagccatggacagagccttgaatggattggaggtattcatcctaacaatggtggtactatctacaaccagaagttcaagggcagggccacattgactgtagacaagtcctccagcacagcctacatggagttccgcagcctgacatctgatgattctgcagtctattactgtgcaagtccctgctggggtcaaggaacctcagtcaccgtctcctca

SEQ ID NO: 39 - DNA, 336 bp, synthetic construct
  source 1..336: mol_type = other DNA
gatgttgtgatgacccagactccactcactttgtcggtttccattggacaaccagcctccatctcttgcaagtcaagtcagagcctcttacatgatgatggaaagacatatttgaattggttgttacagaggccaggccagtctccaaagcgcctgatctctctggtgtctaaactggactctaaattccctgacaggttcactggcagtggatcagggacagatttcacactgaaaatcagcagagtggaggctgaggatttgggagtttattattgctggcaaggtacacattttcctcacacgttcggtgctgggaccaagctggagctgaaa

SEQ ID NO: 40 - protein, 115 aa, synthetic construct
DVKLEDSGGGLVQPEGSMKLSCVASGFIFSDAWMAWVRQSPEKGLEWVAEIRGRASSHATYYAESVKGRFTISRDDSKSSVYLQMNSLRAEDTGVYYCTRGVNYWGQGTALTVSS

SEQ ID NO: 41 - protein, 5 aa, synthetic construct
DAWMA

SEQ ID NO: 42 - protein, 19 aa, synthetic construct
EIRGRASSHATYYAESVKG

SEQ ID NO: 43 - protein, 4 aa, synthetic construct
GVNY

SEQ ID NO: 44 - protein, 112 aa, synthetic construct
DVVMTQIPLTLSVTIGQPASISCKSSQSLLDSDGKTYLIWLLQRPGQSPKRLIYLVSKKDSGVPDRFTGSGSGTDFTLKISRVEAEDLGVYYCWQGTHFPWTFGGGTKLEIK

SEQ ID NO: 45 - protein, 16 aa, synthetic construct
KSSQSLLDSDGKTYLI

SEQ ID NO: 46 - protein, 7 aa, synthetic construct
LVSKKDS

SEQ ID NO: 47 - protein, 9 aa, synthetic construct
WQGTHFPWT

SEQ ID NO: 48 - DNA, 345 bp, synthetic construct
  source 1..345: mol_type = other DNA
gatgtgaagcttgaggactctggaggaggcttggtgcaacctgaaggatccatgaaactctcttgtgttgcctctggattcatttttagtgacgcctggatggcctgggtccgccagtctccagaaaaggggcttgagtgggttgctgaaattagaggcagagctagtagtcatgcaacatactatgctgagtctgtgaaagggaggttcaccatctcaagagatgattccaaaagtagtgtctacctgcaaatgaacagcttaagagctgaagacactggcgtttattactgtaccaggggagtaaactactggggccaaggcaccgctctcacagtctcctca

SEQ ID NO: 49 - DNA, 336 bp, synthetic construct
  source 1..336: mol_type = other DNA
gatgttgtgatgacccagattccactcactttgtcggttaccattggacaaccagcctccatctcttgcaagtcaagtcagagcctcttagatagtgatggaaagacatatttgatttggttattacagaggccaggccagtctccaaagcgcctaatctatctggtgtctaaaaaggactctggagtccctgacaggttcactggcagtggatcagggacagatttcacactgaaaatcagcagagtggaggctgaggatctgggagtttattattgctggcaaggaacacattttccgtggacgttcggtggaggcaccaagctggaaatcaaa

SEQ ID NO: 50 - protein, 116 aa, synthetic construct
QVQLQQSGAELVRPGASVKLSCKALDYTFTDYEMHWVKQTPVHGLEWIGAVHPRSGGTVYNQRFRAKATLTADKSSSTAFIELSSLTSEDSAVYFCTYYGYDEIYWGQGTLVTVSA

SEQ ID NO: 51 - protein, 5 aa, synthetic construct
DYEMH

SEQ ID NO: 52 - protein, 17 aa, synthetic construct
AVHPRSGGTVYNQRFRA

SEQ ID NO: 53 - protein, 7 aa, synthetic construct
YGYDEIY

SEQ ID NO: 54 - protein, 112 aa, synthetic construct
DVVMTQISFSLPVSLGDQASISCRSSQSLVHSDGTTYLHWYLQKPGQSPKLLIYKVSNRFSGVPDRFSGSGSGTDFTLKISRVEAEDLGVYFCSQSTHAPYTFGGGTKLEIK

SEQ ID NO: 55 - protein, 16 aa, synthetic construct
RSSQSLVHSDGTTYLH

SEQ ID NO: 56 - protein, 7 aa, synthetic construct
KVSNRFS

SEQ ID NO: 57 - protein, 9 aa, synthetic construct
SQSTHAPYT

SEQ ID NO: 58 - DNA, 348 bp, synthetic construct
  source 1..348: mol_type = other DNA
caggttcaactgcagcagtctggggctgagctggtgaggcctggggcttcagtgaagctgtcctgcaaggctttggactacacatttactgactatgagatgcactgggtgaagcagacacctgtacatggcctggaatggattggagcggttcatccacgaagtggtggtactgtctataatcagagattcagggccaaggccacactgactgctgacaaatcctccagcacagccttcatagagctcagcagcctgacatctgaagactctgcagtctatttctgtacctactatggttacgacgaaatttattggggccaagggactctggtcactgtctctgca

SEQ ID NO: 59 - DNA, 336 bp, synthetic construct
  source 1..336: mol_type = other DNA
gatgttgtgatgacccaaatttcattctccctgcctgtcagtcttggagatcaagcctccatctcttgcagatctagtcagagccttgtacacagtgatggaaccacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgttctcaaagtacacatgctccgtacacgttcggaggggggaccaagctggaaataaaa

(19) United States
(12) Patent Application Publication
(10) Pub. No.: US 2026/0285956 A1
(43) Pub. Date: Sep. 24, 2026

(54) ANTI-TDP-43 BINDING MOLECULES AND USES THEREOF

(71) Applicant: AC Immune SA, Lausanne (CH)

(72) Inventors: Tariq Afroz, Lausanne (CH); Tamara Seredenin, Lausanne (CH); Elodie Chevalier, Lausanne (CH)

(21) Appl. No.: 19/161,967

(22) PCT Filed: Mar. 7, 2024
(86) PCT No.: PCT/EP2024/056105

§ 371 Date: Sep. 3, 2025

(30) Foreign Application Priority Data
Mar. 8, 2023 (EP) 23160832.4

(51) Int. Cl. C07K 16/18; A61K 39/00; A61K 45/06; A61K 47/68; A61P 25/28; C12N 15/86; G01N 33/58; G01N 33/68

(52) U.S. Cl. CPC C07K 16/18; A61K 45/06; A61K 47/6843; A61P 25/28; C12N 15/86; G01N 33/58; G01N 33/6896; A61K 2039/505; C07K 2317/34; C07K 2317/76; C07K 2317/92; C12N 2750/14143; C12N 2750/14145; G01N 2800/2821

(57) ABSTRACT

The present invention is in the field of transactive response DNA binding protein with a molecular weight of 43 kDa (TARDB or also TDP-43). The invention relates to TDP-43 specific binding molecules, in particular to anti-TDP-43 antibodies or antigen-binding fragment or a derivative thereof and uses thereof. The present invention provides means and methods to diagnose, prevent, alleviate and/or treat a disease, disorder and/or abnormality associated with TDP-43 aggregates including but not limited to Frontotemporal dementia (FTD), amyotrophic lateral sclerosis (ALS), Alzheimer's disease (AD), Parkinson's disease (PD), Chronic Traumatic Encephalopathy (CTE), and limbic-predominant age-related TDP-43 encephalopathy (LATE).

### FIELD OF THE INVENTION

The present invention is in the field of transactive response DNA binding protein with a molecular weight of 43 kDa (TARDB or also TDP-43). The invention relates to TDP-43 specific binding molecules, in particular to anti-TDP-43 antibodies or an antigen-binding fragment or a derivative thereof and uses thereof. The present invention provides means and methods to diagnose, prevent, alleviate and/or treat a disease, a disorder and/or abnormality associated with TDP-43, in particular associated with TDP-43 aggregates, or TDP-43 proteinopathy, including but not limited to Frontotemporal dementia (FTD), amyotrophic lateral sclerosis (ALS), Alzheimer's disease (AD), Parkinson's disease (PD), Chronic Traumatic Encephalopathy (CTE), and limbic-predominant age-related TDP-43 encephalopathy (LATE).

### BACKGROUND

Age-associated brain disorders characterised by pathological aggregation of proteins in the central nervous system (CNS) (proteinopathies) and peripheral organs represent one of the leading causes of disability and mortality in the world. The best characterised protein that forms aggregates is amyloid beta in Alzheimer's disease and related disorders. Other disease-associated, aggregation-prone proteins leading to neurodegeneration include but are not limited to tau, alpha-synuclein (aSyn, a-syn), huntingtin, fused in sarcoma (FUS), dipeptide repeat proteins (DPRs) produced by unconventional translation of the C9orf72 repeat expansion, superoxide dismutase 1 (SOD1), and TDP-43. Diseases involving TDP-43 aggregates are generally listed as TDP-43 proteinopathies including, but not limited to, ALS and FTD.

### I. TDP-43 Introduction

Transactive response (TAR) DNA binding protein 43 kDa (TDP-43) is a 414-amino acid protein encoded by the TARDBP gene on chromosome 1p36.2 (ALS10). TARDBP is comprised of six exons (exon 1 is non-coding; exons 2-6 are protein-coding). TDP-43 belongs to the family of heterogeneous ribonucleoprotein (hnRNP) RNA binding proteins (Wang et al., Trends in Molecular Medicine Vol. 14 No. 11, 2008, 479-485; Lagier-Tourenne et al., Human Molecular Genetics, 2010, Vol. 19, Review Issue 1 R46-R64). TDP-43 contains five functional domains (FIG. 1 in Warraich et al., The International Journal of Biochemistry & Cell Biology 42 (2010) 1606-1609): two RNA recognition motifs (RRM1 and RRM2), which have two highly conserved hexameric ribonucleoprotein 2 (RNP2) and octameric ribonucleoprotein 1 (RNP1) regions, a nuclear export signal (NES) and a nuclear localization signal (NLS) enabling it to shuttle between the nucleus and the cytoplasm transporting bound mRNA, and a glycine-rich domain at the C-terminal, which mediates protein-protein interactions. TDP-43 is involved in multiple aspects of RNA processing, including transcription, splicing, transport, and stabilization (Buratti and Baralle, FEBS Journal 277 (2010) 2268-2281). It is a highly conserved, ubiquitously expressed protein with a tightly autoregulated expression level that shuttles continuously between the nucleus and cytoplasm, but is predominantly localized to the nucleus. In 2006, TDP-43 was identified as the protein that accumulates in the vast majority of cases of frontotemporal lobar degeneration (FTLD) with tau-negative, ubiquitin-positive inclusions (then referred to as FTLD-TDP), and in most cases of amyotrophic lateral sclerosis (ALS) (Arai et al., Biochemical and Biophysical Research Communications 351 (2006) 602-611; Neumann et al., Science 314, (2006), 130-133).

Thirty-eight negative-dominant mutations in TDP-43 have been identified in sporadic and familial ALS patients as well as in patients with inherited FTD mainly located in the glycine-rich domain (FIG. 1 in Lagier-Tourenne and Cleveland, Cell 136, 2009, 1001-1004). TDP-43 is inherently aggregation-prone, as shown by sedimentation assays, and this propensity is further increased by some of the ALS-associated TARDBP mutations (Ticozzi et al., CNS Neurol. Disord. Drug Targets. 2010, 9(3), 285-296.) connecting TDP-43 aggregation with clinical disease manifestation.

### II. TDP-43 in Neurodegeneration

TDP-43 aggregates have been identified in a growing list of neurodegenerative conditions (Lagier-Tourenne et al., Human Molecular Genetics, 2010, Vol. 19, Review Issue 1 R46-R64), including but not limited to: Frontotemporal dementia (FTD, such as sporadic or familial with or without motor-neuron disease (MND), with progranulin (GRN) mutation, with C9orf72 mutations, with TARDBP mutation, with valosin-containing protein (VCP) mutation, linked to chromosome 9p, corticobasal degeneration, frontotemporal lobar degeneration (FTLD) with ubiquitin-positive TDP-43 inclusions (FTLD-TDP), Argyrophilic grain disease, Pick's disease, semantic variant Primary Progressive Aphasia (svPPA), behavioural variant FTD (bvFTD), nonfluent variant Primary Progressive Aphasia (nfvPPA) and the like), Amyotrophic lateral sclerosis (ALS, such as sporadic ALS, with TARDBP mutation, with angiogenin (ANG) mutation), Alexander disease (AxD), limbic-predominant age-related TDP-43 encephalopathy (LATE), Chronic Traumatic Encephalopathy (CTE), Perry syndrome, Alzheimer's disease (AD, including sporadic and familial forms of AD), Down syndrome, Familial British dementia, Polyglutamine diseases (Huntington's disease and spinocerebellar ataxia type 3 (SCA3; also known as Machado Joseph Disease)), Hippocampal sclerosis dementia and Myopathies (sporadic inclusion body myositis, Inclusion body myopathy with a mutation in the valosin-containing protein ((VCP); associated with Paget disease of bone and frontotemporal dementia), Oculo-pharyngeal muscular dystrophy with rimmed vacuoles, Myofibrillar myopathies with mutations in the myotilin (MYOT) gene or mutations in the gene coding for desmin (DES)), Traumatic Brain Injury (TBI), Dementia with Lewy Bodies (DLB) or Parkinson's Disease (PD). The term LATE is intended to encompass several previously used designations related to TDP-43 proteinopathy that may be associated with cognitive impairment, including hippocampal sclerosis, hippocampal sclerosis of ageing, hippocampal sclerosis dementia, cerebral age-related TDP-43 with sclerosis (CARTS), and TDP-43 pathologies in the elderly (for reviews see Kuslansky et al., 2004; Lippa and Dickson, 2004; Nelson et al., 2013, 2016b; Dutra et al., 2015).

Aggregated TDP-43 from patient brains shows a number of abnormal modifications, including hyperphosphorylation, ubiquitination, acetylation and C-terminal fragments through proteolytic cleavage (Arai et al., Biochemical and Biophysical Research Communications 351 (2006) 602-611; Neumann et al., Science 314, (2006), 130-133; Neumann et al., Acta Neuropathol. (2009) 117: 137-149; Hasegawa et al., (2008) Annals of Neurology Vol 64 No 1, 60-70; Cohen et al., Nat Commun. 6: 5845, 2015). The TDP-43 aggregation mechanisms and the TDP-43 species involved in aggregation are not fully understood. However, evidence points to the importance of the C-terminal region of TDP-43 in pathological mechanisms. The C-terminal domain, known as the low complexity domain (LCD), is intrinsically disordered and contains regions enriched in glycine, hydrophobic residues, glutamine and asparagine (Afroz et al., 2019). While this region of TDP-43 has intrinsic properties to form higher-order physiological assemblies such as stress granules (Gasset-Rosa et al., 2019), in disease, irreversible inter- and intra-molecular interactions within this region can result in pathologic aggregates (Gasset-Rosa et al., 2019). In fact, recent structural and biochemical studies have demonstrated that the region composed of amino acids 272-360 of TDP-43, within the C-terminal region, adopts a stable protease-resistant amyloid core structure in the brain of ALS and FTD patients (Arseni et al., 2021; Arseni et al., 2023). Moreover, the disease-specific proteolytic cleavage exposing this amyloid-core is shown to further enhance its seeding activity important for templated aggregation (Kumar et al., 2023). Such proteolytic processing of TDP-43 and their enrichment in patient brains is a disease-specific pathological signature in addition to the aforementioned post-translation modifications.

Another characteristic feature of TDP-43 pathology is redistribution and accumulation of TDP-43 from nucleus to cytoplasm. The hallmark lesions of FTLD-TDP are neuronal and glial cytoplasmic inclusions (NCI and GCI, respectively) and dystrophic neurites (DN) that are immunoreactive for TDP-43, as well as ubiquitin and p62, but negative for other neurodegenerative disease-related proteins. Differences in inclusion morphology and tissue distribution thereof are associated with specific mutations and/or clinical representations. Four types of TDP-43 pathology are described so far by histological classification (Mackenzie and Neumann, J. Neurochem. (2016) 138 (Suppl. 1), 54-70). FTLD-TDP type A cases are characterised by abundant short dystrophic neuritis (DN) and compact oval or crescentic NCI, predominantly in layer II of the neocortex (FIG. 2f in Mackenzie et al., 2016 J. Neurochem. 138 (Suppl. 1), 54-70). Cases with this pathology usually present clinically with either behavioural-variant frontotemporal dementia (bvFTD) or nonfluent/agrammatic variants of Primary Progressive Aphasia (nfvPPA) and are associated with progranulin (GRN) mutations. Type B cases show moderate numbers of compact or granular NCI in both superficial and deep cortical layers with relatively few DN and NII (neuronal intranuclear inclusions; FIG. 2g in Mackenzie et al., 2016 J. Neurochem. 138 (Suppl. 1), 54-70). Most cases with co-appearance of FTD and ALS symptoms are found to have FTLD-TDP type B pathology. Type C cases have an abundance of long, tortuous neurites, predominantly in the superficial cortical laminae, with few or no NCI (FIG. 2j in Mackenzie et al., 2016 J. Neurochem. 138 (Suppl. 1), 54-70). This pathology is particularly found in cases presenting with semantic variant of Primary Progressive Aphasia (svPPA). FTLD-TDP type D displays with abundant lentiform neuronal intranuclear inclusions (NII) and short DN in the neocortex with only rare NCI (FIG. 2k in Mackenzie et al., 2016 J. Neurochem. 138 (Suppl. 1), 54-70). Type E is characterised by granulofilamentous neuronal inclusions (GFNIs) and very fine, dot-like neuropil aggregates affecting all neocortical layers in addition to curvilinear oligodendroglial inclusions in the white matter (Edward B. Lee et al., Acta Neuropathol. 2017 July; 134(1): 65-78.). This pattern of pathology is only found in cases with VCP in association with inclusion body myositis.

### III. TDP-43 in FTD

Frontotemporal dementia (FTD) is a clinical term that covers a wide spectrum of disorders based on the degeneration of frontal and temporal lobes—a pathological feature termed frontotemporal lobar degeneration (FTLD). FTD is the second most abundant cause of early degenerative dementias in the age group below 65 years (Le Ber, Revue Neurologique 169 (2013) 811-819). FTD is presented by several syndromes including bvFTD which is characterised by changes in personality and behaviour; semantic dementia (SD) and progressive nonfluent aphasia (PNFA) characterised by changes in the language function; corticobasal syndrome (CBS), progressive supranuclear palsy syndrome and motor neuron disease (FTD-MND) characterised by movement dysfunction. Clinical diagnosis of these syndromes is complicated and final conclusion can only be achieved through post-mortem histopathological analysis to detect aggregated protein and define affected brain regions. In terms of pathological, proteinaceous inclusions, about 45% of cases show pathological accumulation of misfolded tau, 45% of cases have pathological TDP-43 and a smaller subgroup has aggregates of FUS and other proteins.

### IV. TDP-43 in ALS

Amyotrophic lateral sclerosis (ALS) is a neurodegenerative disorder characterised by the premature loss of upper and lower motor neurons. The progression of ALS is marked by fatal paralysis and respiratory failure with a disease course from diagnosis to death of 1 to 5 years. In most cases of sporadic ALS, the neuropathology is characterised by abnormal cytoplasmic accumulations of TDP-43 in neurons and glia of the primary motor cortex, brainstem motor nuclei, spinal cord and the associated white matter tracts. ALS with dementia involves accumulation of TDP-43 in extramotor neocortex and hippocampus. The role of phosphorylation of TDP-43 in ALS patients has been explored with the help of antibodies that specifically bind to phosphorylated TDP-43 in nuclear and cytoplasmic inclusions with amino acids S379, S403, S404, S409, S410 as the major sites of phosphorylation of TDP-43 (Hasegawa et al., Ann Neurol 2008; 64: 60-70; Neumann et al., Acta Neuropathol (2009) 117: 137-149).

### V. TDP-43 in AD and Other Diseases

TDP-43 pathology occurs in up to 57% of brains of patients with Alzheimer's disease (Josephs K A et al., Acta Neuropathol. 2014; 127(6): 811-824; Josephs K A et al., Acta Neuropathol. 2014; 127(3): 441-450; McAleese et al., Brain Pathol. 2017 July; 27(4): 472-479). TDP-43 aggregation is associated with patient's age and correlates with cognitive decline, memory loss and medial temporal atrophy in AD. It appears that in AD, TDP-43 represents a secondary or independent pathology that shares overlapping brain distribution with amyloid beta and tau pathologies in the medial temporal lobe. Pathologic TDP-43 follows a stereotypical pattern of progressive deposition that has been described by the so-called TDP-43 in AD (TAD) staging scheme: TDP-43 first deposits in the amygdala (stage I) followed by hippocampus, limbic, temporal, and finally the frontostriatum (stage V) (Josephs K A et al., Acta Neuropathol. 2014; 127(6): 811-824; Josephs K A et al., Acta Neuropathol. 2014; 127(3): 441-450).

### VI. TDP-43 Spreading

Although ALS and FTD onset and first symptoms vary significantly between patients, the common feature of disease progression is spreading of pathology from an initial focal area to anatomically connected brain regions. The continuous worsening of symptoms might be explained by the progressive spread of TDP-43 pathology. TDP-43 pathology in an ALS patient's brain appears to be spreading in a four-stage process and it is believed that propagation occurs transynaptically via corticofugal axonal projections using anterograde axonal transport (Brettschneider et al., Ann Neurol. 2013 July; 74(1): 20-38.). Recent experimental evidence supports the hypothesis of protein propagation in neuronal tissue for amyloid-beta, tau, alpha-synuclein and TDP-43 by a prion-like mechanism (Hasegawa et al., 2017), with starting points and the topographical spreading patterns being distinct for the four proteins (Brettschneider J et al., Nature Rev. Neuroscience, 2015, 109). The common, disease unifying mechanism is believed to be based on the cell-to-cell spreading of pathological protein aggregates. This mechanism consists of the release of aggregates from a diseased cell, uptake by a naïve cell and seeding of the pathological protein conformation by a templated conformational change of endogenous proteins. Pathological TDP-43 able to induce aggregation of physiological (i.e., non-pathological TDP-43) is defined as seeding-competent TDP-43. Indeed, TDP-43 has been found to misfold and aggregate into seeds that are propagating agents with the ability to trigger de novo misfolding. This “prion-like” paradigm is suspected to be one of the key elements in the disease progression.

TDP-43 cell-to cell spreading has been studied at a molecular level in few in vitro models, where insoluble TDP-43 preparations from patient brain are able to induce intracellular aggregate formation in receptor cells (Nonaka et al., Cell Reports 4 (2013), 124-134; Feiler et al., 2015; Porta et al., Nat. Comm., 2018). Further, it has been observed that intracellular TDP-43 aggregates are released in association with exosome prior to spreading to the next cell (Nonaka et al., Cell Reports 4 (2013, 124-134)). Similarly, adenovirus-transduced TDP-43 expression led to cytoplasmic aggregates which were phosphorylated, ubiquitinated and more importantly acted as seeds initiating cell-to-cell spreading (Ishii et al., PLoS ONE 12(6): e0179375, 2017). The patient-derived pathological TDP-43 can lead to widespread deposition of endogenous TDP-43 following intracerebral inoculation into transgenic and wildtype mice (Porta et al., Nat. Comm., 2018). The presence of TDP-43 seeding-competent species in CSF or ALS patients was recently confirmed using a TDP-43 seed amplification assay (Audrain et al., 2023). A TDP-43 mAb targeting the C-terminal domain was able to neutralize these seeding species (Audrain et al., 2023).

### VII. Prevention and Treatment of TDP-43 Proteinopathies

TDP-43 aggregation and spreading of pathology are major hallmarks of ALS and FTD—fatal diseases for which currently no cure is available. Therefore, there is a need for new methods for the treatment and prevention of TDP-43 proteinopathies. Mutations in TDP-43 are associated with familial cases of ALS and FTD providing a causative link between TDP-43 misfolding and disease progression.

### VIII. Diagnosis of TDP-43 Proteinopathies

The diagnosis of FTD based on clinical manifestations is insufficient since the clinical representation can overlap with other diseases, in particular in the earlier stages.

A number of approaches aims at the development of biochemical biomarkers to distinguish different types of FTD pathology. Development of antibodies against different conformations of TDP-43 may permit generating more sensitive and specific diagnostic tools. In parallel to biochemical biomarkers, the development of imaging biomarkers may enable early and specific detection of the pathology in TDP-43 proteinopathies. The ability to image TDP-43 deposition in the brain may be a substantial achievement for diagnosis and drug development for TDP-43 proteinopathies. Using cell permeable antibody fragments could enable such detection.

The earliest event in neurodegenerative diseases based on misfolding of different proteins is the acquisition of an alternative conformation that renders the protein toxic. Moreover, this misfolded conformation can self-propagate by recruiting the endogenous, normal protein into the misfolded conformation as mechanistic basis for the observed spread through affected tissue.

To develop antibodies against different conformational states of a given protein, supramolecular antigenic constructs were designed in which the conformation of the presented antigen was controlled to raise conformational-specific antibodies against a given target in a specific conformational state (WO2012/055933 and WO2012/020124). Conformational-specific antibodies offer many advantages since they can discriminate between the disease-associated and the functional, endogenous conformation of these proteins. This approach offers many advantages in the therapeutic application since such antibodies are less likely to be adsorbed by the normal conformation of proteins while targeting the misfolded, disease associated isoform thereof. Similar to this, for diagnostic application such antibodies only recognize the disease-associated, structural state of a protein, which is paramount for the development of the sensitive and specific diagnostics.

The use of a TDP-43-based biomarker in TDP-43 proteinopathies still remains to be established. Such evaluation has been hindered in part due to the lack of high affinity antibodies that can be employed in a suitable immunoassay for quantification of pathological TDP-43 in biofluids (Feneberg et al., Molecular Neurobiology, 2018).

Therefore, there is a clear need for biomarkers able to detect misfolded aggregated TDP-43 and non-aggregated physiological TDP-43, in particular in a human sample, for diagnosing different types of TDP-43 proteinopathies and/or for monitoring efficacy of therapeutic drugs used for treatment of diseases, disorders and abnormalities associated with TDP-43, in particular associated with TDP-43 aggregates or TDP-43 proteinopathy.

The TDP-43 proteinopathies are defined as a set of neurodegenerative disorders characterised by pathological TDP-43.

### IX. Prior Art

Patent application WO2008/151055 discloses methods and materials for using the levels of TDP-43 polypeptides and/or TDP-43 polypeptide cleavage products (e.g. 25 kD and 35 kD TDP-43 polypeptide cleavage products) in a biological fluid to determine whether or not a mammal has a neurodegenerative disease.

Patent application WO2013/061163 discloses TDP-43 specific binding molecules including polypeptides such as human antibodies as well as fragments, derivatives and variants thereof. Patent application WO2020/234473 discloses specific binding molecules including polypeptides such as murine antibodies or antigen-binding fragments thereof.

Patent application WO2022/034228 discloses TDP-43 specific binding molecules including polypeptides such as humanized antibodies or antigen-binding fragments thereof.

In view of the foregoing, there is a need for anti-TDP-43 binding molecules which bind misfolded aggregated TDP-43 and non-aggregated physiological TDP-43, particularly human TDP-43 (SEQ ID NO: 1).

### SUMMARY

At present there are no approved therapies on the market to treat and/or prevent TDP-43 associated diseases. There is therefore a pressing need to identify new therapies that can treat and/or prevent these diseases. Accordingly, the invention relates to binding molecules, in particular antibodies or antigen-binding fragments thereof, which specifically recognize misfolded aggregated TDP-43 and non-aggregated physiological TDP-43. Within the invention, misfolded TDP-43 includes misfolded monomeric and/or misfolded oligomeric and/or misfolded aggregated and/or post-translationally modified and/or misfolded truncated TDP-43. Post-translationally modified TDP-43 comprises phosphorylated, ubiquitylated, acetylated, sumoylated, and/or methylated TDP-43. Physiological TDP-43 includes soluble nuclear TDP-43. It is demonstrated herein that the binding molecules of the invention are capable of binding pathological TDP-43, including TDP-43 aggregates and phosphorylated TDP-43. Thus, the invention provides binding molecules, in particular antibodies or antigen-binding fragments thereof, which specifically recognize misfolded aggregated TDP-43 and non-aggregated physiological TDP-43. Such binding molecules are referred to herein as “pan-TDP-43” binding molecules, in particular pan-TDP-43 antibodies. As explained herein, the TDP-43 binding molecules of the invention may bind misfolded aggregated TDP-43 and non-aggregated physiological TDP-43 equally, or to one preferentially to the other whilst binding to both categories of TDP-43 specifically. The invention also provides the binding molecules, in particular antibodies or antigen-binding fragments thereof, for the prevention, alleviation, treatment and/or diagnosis of diseases, disorders and abnormalities associated with TDP-43, in particular associated with TDP-43 aggregates, or TDP-43 proteinopathy. The invention also provides the binding molecules, in particular antibodies or antigen-binding fragments thereof, for detecting and/or understanding (i.e. identifying) the specific type of pathology causing neurodegeneration. Envisaged are uses as diagnostic biomarkers enabling more efficient and precise subject selection for longitudinal monitoring in clinical studies, supporting the development of novel therapeutics for TDP-43 proteinopathies.

The invention also provides the TDP-43 binding molecules, in particular antibodies or antigen-binding fragments thereof, as a medicine (therapeutic agent).

Without wishing to be bound by any particular theory, the present invention was developed based on the assumption that modified conformation-specific antigenic peptides and peptide fragments derived from TDP-43 protein or the whole TDP-43 protein and the antibodies obtainable or obtained by using said peptides or fragments or the whole TDP-43 protein as antigen block TDP-43 cell-to-cell propagation, and/or disaggregate TDP-43 aggregates and/or block TDP-43 seeding and/or neutralize seeding-competent TDP-43 and/or inhibit the aggregation of TDP-43 protein or fragments thereof and/or potentiate TDP-43 clearance. The binding molecules of the invention, in particular polypeptides, more particularly antibodies or antigen-binding fragments thereof, bind to misfolded aggregated TDP-43, particularly to cytoplasmic and extracellular misfolded TDP-43. The humanized binding molecules of the invention, in particular polypeptides, more particularly antibodies or antigen-binding fragments thereof, bind to full-length TDP-43 and/or truncated TDP-43. In one embodiment, the binding molecules of the invention, in particular polypeptides, more particularly antibodies or antigen-binding fragments thereof, specifically bind to cytoplasmic misfolded TDP-43. In one embodiment, the TDP-43 binding molecules of the invention, in particular antibodies or antigen-binding fragments thereof, bind and neutralize seeding-competent TDP-43. In one embodiment, the TDP-43 binding molecules of the invention, in particular antibodies or antigen-binding fragments thereof, bind extracellular and/or aggregated TDP-43 and potentiates its clearance by immune cells such as microglia through antibody-dependent cellular phagocytosis (ADCP).

Misfolded aggregated, or pathology-associated, TDP-43 is composed of TDP-43 proteins that lose their normal folding (i.e. are misfolded) and localization. Misfolded aggregated TDP-43 can be found in preinclusions and in neuronal and glial cytoplasmic inclusions (NCI and GCI, respectively), neuronal intranuclear inclusions (NII) and dystrophic neurites (DN) that are immunoreactive for TDP-43.

Non-aggregated physiological TDP-43 is physiologically functional TDP-43 protein predominantly located in the nucleus and shuttling to the cytoplasm, being in a status able to exhibit its desired function in an in vivo cellular environment.

The TDP-43 binding molecules of the invention, in particular the anti-TDP-43 antibodies or antigen-binding fragments thereof, surprisingly have at least one, preferably two, more preferably three, even more preferably four, still more preferably five, still even more preferably six, still even more preferably seven, most preferably all eight of the following characteristics:
blocks TDP-43 cell-to-cell propagation;
disaggregates TDP-43 aggregates;
inhibits the aggregation of TDP-43 protein or fragments thereof;
blocks TDP-43 seeding;
neutralizes seeding-competent TDP-43;
blocks TDP-43 spreading;
potentiates TDP-43 clearance; and
reduces phosphorylated TDP-43 level in vivo.

Independent of the combination of one, two, three, four, five, six, seven or eight above listed characteristics, the anti-TDP-43 binding molecules, preferably anti-TDP-43 antibodies or antigen-binding fragments thereof, of the invention may decrease phosphorylated TDP-43 level in the brain and/or ameliorate/inhibit/reduce the formation of TDP-43 pathology in an in vivo model of TDP-43 proteinopathies and more importantly in patients with TDP-43 pathology.

The anti-TDP-43 binding molecules bind to an epitope within amino acids 304-414 of human TDP-43 (SEQ ID NO: 1). The anti-TDP-43 binding molecules bind to an epitope comprising, consisting essentially of or consisting of amino acids residues 304-313, 356-361 or 397-407 of human TDP-43 (SEQ ID NO: 1). Alternatively, the anti-TDP-43 binding molecules may bind to an epitope within amino acid residues 396-414 of human TDP-43 (SEQ ID NO: 1). According to the invention there is provided a TDP-43 binding molecule, in particular a TDP-43 antibody or an antigen-binding fragment thereof, comprising:
a. a Heavy Chain Variable Region (VH) which comprises a VH-CDR1 comprising the amino acid sequence of SEQ ID NO: 51, a VH-CDR2 comprising the amino acid sequence of SEQ ID NO: 52 and a VH-CDR3 comprising the amino acid sequence of SEQ ID NO: 53; and a Light Chain Variable Region (VL) which comprises a VL-CDR1 comprising the amino acid sequence of SEQ ID NO: 55, a VL-CDR2 comprising the amino acid sequence of SEQ ID NO: 56 and a VL-CDR3 comprising the amino acid sequence of SEQ ID NO: 57; or
b. a Heavy Chain Variable Region (VH) which comprises a VH-CDR1 comprising the amino acid sequence of SEQ ID NO: 41, a VH-CDR2 comprising the amino acid sequence of SEQ ID NO: 42 and a VH-CDR3 comprising the amino acid sequence of SEQ ID NO: 43; and a Light Chain Variable Region (VL) which comprises a VL-CDR1 comprising the amino acid sequence of SEQ ID NO: 45, a VL-CDR2 comprising the amino acid sequence of SEQ ID NO: 46 and a VL-CDR3 comprising the amino acid sequence of SEQ ID NO: 47; or
c. a Heavy Chain Variable Region (VH) which comprises a VH-CDR1 comprising the amino acid sequence of SEQ ID NO: 31, a VH-CDR2 comprising the amino acid sequence of SEQ ID NO: 32 and a VH-CDR3 comprising the amino acid sequence PC (Pro-Cys); and a Light Chain Variable Region (VL) which comprises a VL-CDR1 comprising the amino acid sequence of SEQ ID NO: 35, a VL-CDR2 comprising the amino acid sequence of SEQ ID NO: 36 and a VL-CDR3 comprising the amino acid sequence of SEQ ID NO: 37; or
d. a Heavy Chain Variable Region (VH) which comprises a VH-CDR1 comprising the amino acid sequence of SEQ ID NO: 21, a VH-CDR2 comprising the amino acid sequence of SEQ ID NO: 22 and a VH-CDR3 comprising the amino acid sequence of SEQ ID NO: 23; and a Light Chain Variable Region (VL) which comprises a VL-CDR1 comprising the amino acid sequence of SEQ ID NO: 15, a VL-CDR2 comprising the amino acid sequence of SEQ ID NO: 26 and a VL-CDR3 comprising the amino acid sequence of SEQ ID NO: 17; or
e. a Heavy Chain Variable Region (VH) which comprises a VH-CDR1 comprising the amino acid sequence of SEQ ID NO: 11, a VH-CDR2 comprising the amino acid sequence of SEQ ID NO: 12 and a VH-CDR3 comprising the amino acid sequence of SEQ ID NO: 13; and a Light Chain Variable Region (VL) which comprises a VL-CDR1 comprising the amino acid sequence of SEQ ID NO: 15, a VL-CDR2 comprising the amino acid sequence of SEQ ID NO: 16 and a VL-CDR3 comprising the amino acid sequence of SEQ ID NO: 17.

The invention is further directed, inter alia, to (i) an immunoconjugate comprising the TDP-43 binding molecule, (ii) a labelled antibody comprising the TDP-43 binding molecule, (iii) a pharmaceutical composition comprising the TDP-43 binding molecule and a pharmaceutically acceptable carrier and/or excipients and/or diluents, (iv) a TDP-43 binding molecule for human or veterinary medicine use, (v) a TDP-43 binding molecule for use in the prevention, alleviation, treatment of diseases, disorders and/or abnormalities associated with TDP-43 or TDP-43 proteinopathy, (vi) a TDP-43 binding molecule for diagnostic use (in particular for in vivo diagnosis, but also for in vitro testing), (vii) a TDP-43 binding molecule for research use, in particular as an analytical tool or reference molecule, (viii) a TDP-43 binding molecule for use as a diagnostic tool to monitor diseases, disorders and/or abnormalities associated with TDP-43 or TDP-43 proteinopathy, (ix) a method of retaining or increasing cognitive memory capacity or slowing memory loss in an individual with a disease, disorder and/or abnormality associated with TDP-43 or a TDP-43 proteinopathy by treating the individual with a TDP-43 binding molecule, (x) a method of reducing the level of aggregated TDP-43 and/or phosphorylated TDP-43 in an individual by treating the individual with a TDP-43 binding molecule, (xi) a nucleic acid molecule encoding the TDP-43 binding molecule, (xii) a recombinant expression vector comprising a nucleic acid molecule of the present invention, (xiii) a host cell comprising the nucleic acid and/or the vector of present invention, (xiv) a cell-free expression system containing the recombinant expression vector of present invention, (xv) a method for producing a TDP-43 binding molecule, (xvi) a method of quantifying TDP-43 in a sample obtained from a subject using a TDP-43 binding molecule, and (xvii) a kit comprising TDP-43 binding molecules of the invention and/or nucleic acids, expression vectors, host cells and/or cell free expression systems for producing the same.

The TDP-43 binding molecules of the invention, in particular the anti-TDP-43 antibodies or antigen-binding fragments thereof, may recruit and/or activate microglia. More specifically, the TDP-43 binding molecules of the invention may affect microglial morphology in terms of cell size and activation state. This may contribute to the reduction of TDP-43 pathology demonstrated by the TDP-43 binding molecules of the invention.

In the present invention, the binding molecules, in particular antibodies or antigen-binding fragments thereof, specifically recognize TDP-43. The binding molecules of the invention include polypeptides and/or antibodies and/or antigen-binding fragments thereof specific to/for the TDP-43 protein. “Specifically recognize TDP-43” means that the binding molecules of the invention specifically, generally, and collectively, bind to TDP-43, in particular some epitopes within TDP-43, in particular an epitope exposed/accessible in one or more pathological conformation(s) of TDP-43 protein, with greater affinity than for other epitopes. The binding molecules of the invention, in particular polypeptides, more particularly antibodies or antigen-binding fragments thereof, that specifically bind to TDP-43, specifically recognize misfolded aggregated TDP-43 and non-aggregated physiological TDP-43.

The TDP-43 binding molecules of the invention, in particular the antibodies or antigen-binding fragments thereof, bind to both non-aggregated physiological TDP-43 and aggregated TDP-43. Thus, TDP-43 binding molecules of the invention, in particular the antibodies or antigen-binding fragments thereof, may bind approximately equally well to soluble and aggregated TDP-43. TDP-43 binding molecules of the invention, in particular the antibodies or antigen-binding fragments thereof, may bind approximately equally to aggregated TDP-43 as compared with non-aggregated TDP-43. More particularly, TDP-43 binding molecules of the invention, in particular the antibodies or antigen-binding fragments thereof, may bind approximately equally to aggregated TDP-43 in the cytoplasm as compared with non-aggregated TDP-43 in the nucleus. In other embodiments, TDP-43 binding molecules of the invention, in particular the antibodies or antigen-binding fragments thereof, may preferentially bind to aggregated TDP-43 as compared with non-aggregated TDP-43, whilst binding to both species. More particularly, TDP-43 binding molecules of the invention, in particular the antibodies or antigen-binding fragments thereof, may preferentially bind to aggregated TDP-43 in the cytoplasm as compared with non-aggregated TDP-43 in the nucleus, whilst binding to both species. Alternatively, in other embodiments, TDP-43 binding molecules of the invention, in particular the antibodies or antigen-binding fragments thereof, may preferentially bind to non-aggregated TDP-43 as compared with aggregated TDP-43, whilst binding to both species. More particularly, TDP-43 binding molecules of the invention, in particular the antibodies or antigen-binding fragments thereof, may preferentially bind to non-aggregated TDP-43 in the nucleus as compared with aggregated TDP-43 in the cytoplasm, whilst binding to both species. These binding properties may be demonstrated for example using immunohistochemistry.

In some embodiments, the invention encompasses binding molecules, particularly antibodies and antigen-binding fragments thereof of the invention as described herein that specifically bind TDP-43 and the use of these binding molecules to diagnose, prevent, alleviate and/or treat a disease, disorder and/or abnormality associated with TDP-43, in particular associated with TDP-43 aggregates, or TDP-43 proteinopathy including, but not limited to, frontotemporal dementia (FTD), amyotrophic lateral sclerosis (ALS), Alzheimer's disease (AD), Parkinson's disease (PD), Chronic Traumatic Encephalopathy (CTE) and limbic-predominant age-related TDP-43 encephalopathy (LATE). The methods and compositions disclosed herein have applications in diagnosing, preventing, alleviating and/or treating a disease, disorder and/or abnormality associated with TDP-43, in particular associated with TDP-43 aggregates, or TDP-43 proteinopathy including but not limited to frontotemporal dementia (FTD), amyotrophic lateral sclerosis (ALS). Preferably, the use of these binding molecules to diagnose, prevent, alleviate and/or treat a disease, disorder and/or abnormality associated with TDP-43, in particular associated with TDP-43 aggregates, or TDP-43 proteinopathy is directed to amyotrophic lateral sclerosis (ALS), Alzheimer's disease (AD) or Frontotemporal dementia (FTD). More preferably, the use is directed to amyotrophic lateral sclerosis (ALS). More preferably, the use is directed to Alzheimer's disease (AD). More preferably, the use is directed to Frontotemporal dementia (FTD).

In another embodiment, a TDP-43 binding molecule, particularly an anti TDP-43 antibody or an antigen-binding fragment thereof of the invention as described herein specific for TDP-43 is contacted with a sample to detect, diagnose and/or monitor a disease, disorder and/or abnormality associated with TDP-43, in particular associated with TDP-43 aggregates, or TDP-43 proteinopathy, selected from Frontotemporal dementia (FTD, such as sporadic or familial with or without motor-neuron disease (MND), with progranulin (GRN) mutation, with C9orf72 mutations, with TARDBP mutation, with valosin-containing protein (VCP) mutation, linked to chromosome 9p, corticobasal degeneration, frontotemporal lobar degeneration (FTLD) with ubiquitin-positive TDP-43 inclusions (FTLD-TDP), Argyrophilic grain disease, Pick's disease, semantic variant Primary Progressive Aphasia (svPPA), behavioural variant FTD (bvFTD), nonfluent variant Primary Progressive Aphasia (nfvPPA) and the like), Amyotrophic lateral sclerosis (ALS, such as sporadic ALS, with TARDBP mutation, with angiogenin (ANG) mutation), Alexander disease (AxD), limbic-predominant age-related TDP-43 encephalopathy (LATE), Chronic Traumatic Encephalopathy (CTE), Perry syndrome, Alzheimer's disease (AD, including sporadic and familial forms of AD), Down syndrome, Familial British dementia, Polyglutamine diseases (Huntington's disease and spinocerebellar ataxia type 3 (SCA3; also known under Machado Joseph Disease)), Hippocampal sclerosis dementia and Myopathies (sporadic inclusion body myositis, Inclusion body myopathy with a mutation in the valosin-containing protein ((VCP); associated with Paget disease of bone and frontotemporal dementia), Oculo-pharyngeal muscular dystrophy with rimmed vacuoles, Myofibrillar myopathies with mutations in the myotilin (MYOT) gene or mutations in the gene coding for desmin (DES)), Traumatic Brain Injury (TBI), Dementia with Lewy Bodies (DLB) or Parkinson's disease (PD).

In one embodiment, the invention encompasses binding molecules, particularly antibodies or antigen-binding fragments thereof of the invention as described herein that specifically bind TDP-43 and the use of these binding molecules, particularly of these antibodies, to detect the presence of TDP-43 in a sample. Accordingly, TDP-43 binding molecules of the invention, such as anti-TDP43 antibodies as described herein, can be used, inter alia, to screen a clinical sample, in particular human blood, cerebrospinal fluid (CSF), interstitial fluid (ISF) and/or urine for the presence of TDP-43 in samples, for example, by using an ELISA-based or surface adapted assay. Tissue samples may be used in some circumstances, such as brain tissue samples. The methods and compositions of the invention also have applications in diagnosing presymptomatic disease and/or in monitoring disease progression and/or therapeutic efficacy. According to some embodiments, an antibody specific for TDP-43 (e.g., a full-length antibody or a TDP-43 binding fragment or derivative of an antibody) is contacted with a sample (e.g., blood, urine, cerebrospinal fluid (CSF), interstitial fluid (ISF) or brain tissue) to detect, diagnose and/or monitor Frontotemporal dementia (FTD, such as sporadic or familial with or without motor-neuron disease (MND), with progranulin (GRN) mutation, with C9orf72 mutations, with TARDBP mutation, with valosin-containing protein (VCP) mutation, linked to chromosome 9p, corticobasal degeneration, frontotemporal lobar degeneration (FTLD) with ubiquitin-positive TDP-43 inclusions (FTLD-TDP), Argyrophilic grain disease, Pick's disease, semantic variant Primary Progressive Aphasia (svPPA), behavioural variant FTD (bvFTD), nonfluent variant Primary Progressive Aphasia (nfvPPA) and the like), Amyotrophic lateral sclerosis (ALS, such as sporadic ALS, with TARDBP mutation, with angiogenin (ANG) mutation), Alexander disease (AxD), limbic-predominant age-related TDP-43 encephalopathy (LATE), Chronic Traumatic Encephalopathy (CTE), Perry syndrome, Alzheimer's disease (AD, including sporadic and familial forms of AD), Down syndrome, Familial British dementia, Polyglutamine diseases (Huntington's disease and spinocerebellar ataxia type 3 (SCA3; also known under Machado Joseph Disease)), Hippocampal sclerosis dementia and Myopathies (sporadic inclusion body myositis, Inclusion body myopathy with a mutation in the valosin-containing protein ((VCP); associated with Paget disease of bone and frontotemporal dementia), Oculo-pharyngeal muscular dystrophy with rimmed vacuoles, Myofibrillar myopathies with mutations in the myotilin (MYOT) gene or mutations in the gene coding for desmin (DES)), Traumatic Brain Injury (TBI), Dementia with Lewy Bodies (DLB) or Parkinson's disease (PD). The TDP-43 binding molecules of the invention may be used to quantify TDP-43 in suitable samples, in particular clinical samples such as blood, brain tissue, CSF, ISF or urine, with relatively high TDP-43 levels, as compared to a suitable control, indicating disease and/or more advanced disease. Many suitable immunoassay formats are known. Thus, the methods (such as ELISA, MSD (Meso Scale Discovery), HTRF (Homogeneous Time Resolved Fluorescence), SIMOA® (Single Molecule Array) and AlphaLISA®) may be performed for diagnostic purposes with high levels of TDP-43 indicating disease. Alternatively, the methods may be performed for monitoring purposes. Increased levels over time may indicate progression of the disease. Decreased levels over time may indicate regression of the disease. The methods may also be used to monitor therapy, in particular to monitor the efficacy of a particular treatment. Successful therapy may be measured with reference to stable or decreasing levels of TDP-43 following treatment. It is demonstrated in Example 12 of WO2020/234473 that TDP-43 levels were higher in CSF samples from TDP-43 proteinopathy patients than in control samples taken from healthy subjects (healthy control). The control samples may or may not be run in parallel with the test samples. In some embodiments control levels are determined from a series of control samples taken from healthy subjects under similar or the same experimental conditions and used as a comparator for levels determined in the test sample. Methods of quantifying TDP-43 in suitable samples using binding molecules of the invention may also be used to select a therapy (for further treatment of the subject). Thus, personalized treatment methods are envisaged. A sample is taken before and after treatment. If treatment using the therapy results in stable or, preferably, decreasing levels of TDP-43 following treatment the therapy may be selected for that subject. If the therapy does not result in stable or, preferably, decreasing levels of TDP-43 following treatment the therapy is not selected for the subject. The therapy may be any suitable candidate therapeutic agent for treatment of TDP-43 proteinopathies. In preferred embodiments, the therapy comprises a TDP-43 binding molecule of the invention, typically in the form of a pharmaceutical composition as described herein.

The TDP-43 binding molecules of the invention may also be used for disease classification into particular types or subtypes. Thus, there is provided a method for classifying a disease, disorder and/or abnormality associated with TDP-43, in particular associated with TDP-43 aggregates or for classifying a TDP-43 proteinopathy comprising:
a. performing the methods of the invention in which levels of TDP-43 are quantified, as compared to suitable controls,
b. optionally identifying mutations in a sample from the subject including but not limited to progranulin (GRN) mutation, C9orf72 mutations, TARDBP mutation, angiogenin (ANG) mutation, mutation in the valosin-containing protein (VCP), mutation in the myotilin (MYOT) gene or mutations in the gene coding for desmin (DES), and
c. classifying the disease, disorder and/or abnormality associated with TDP-43, in particular associated with TDP-43 aggregates, or TDP-43 proteinopathy.

Similarly, there is provided a method for classifying a disease, disorder and/or abnormality associated with TDP-43, in particular associated with TDP-43 aggregates, or for classifying a TDP-43 proteinopathy comprising: performing the methods of the invention in which levels of TDP-43 are quantified in a sample obtained from a subject with a disease, disorder and/or abnormality associated with TDP-43, or TDP-43 proteinopathy, wherein the levels are compared with control samples taken from subjects with different types or subtypes of disease, disorder and/or abnormality (i.e. a representative set of control levels are determined for the types or subtypes of interest) associated with TDP-43, in particular associated with TDP-43 aggregates, or TDP-43 proteinopathy; and classifying the disease, disorder and/or abnormality associated with TDP-43, in particular associated with TDP-43 aggregates, or TDP-43 proteinopathy based on the comparison. Thus, the classification is based on determining the closest match between the test sample and one or more of the control samples. These methods may further comprise identifying mutations in the sample including but not limited to progranulin (GRN) mutation, C9orf72 mutations, TARDBP mutation, angiogenin (ANG) mutation, mutation in the valosin-containing protein (VCP), mutation in the myotilin (MYOT) gene or mutations in the gene coding for desmin (DES), wherein the identified mutations are also used to classify the disease, disorder and/or abnormality associated with TDP-43, in particular associated with TDP-43 aggregates, or TDP-43 proteinopathy. For the avoidance of doubt, the identification of mutations in a sample may be performed by any suitable method, for example, based on nucleic acid sequencing of nucleic acid molecules within the sample. The sample may be separate and distinct from the sample in which TDP-43 levels are determined, but is from the same subject.

In other embodiments, the invention provides methods for preventing, alleviating and/or treating a disease, disorder and/or abnormality associated with TDP-43, in particular associated with TDP-43 aggregates, or TDP-43 proteinopathy. According to one embodiment, the methods of the invention comprise administering an effective concentration of a binding molecule, particularly an antibody of the invention specific for TDP-43 (e.g., a full-length antibody or a TDP-43 binding fragment or derivative of an antibody) as described herein to a subject. In another embodiment, the invention provides a method for preventing, alleviating and/or treating a TDP-43 proteinopathy. According to some embodiments, a binding molecule, particularly an antibody of the invention or an antigen-binding fragment thereof as described herein specific for TDP-43 is administered to treat, alleviate and/or prevent frontotemporal degeneration (FTD) or amyotrophic lateral sclerosis (ALS). In another embodiment, a binding molecule, particularly an antibody of the invention or an antigen-binding fragment thereof as described herein specific for TDP-43 is administered to prevent, alleviate and/or treat a neurodegenerative disease selected from frontotemporal dementia (FTD), amyotrophic lateral sclerosis (ALS), Alzheimer's disease (AD, including sporadic and familial forms of AD), Parkinson's disease (PD), Chronic Traumatic Encephalopathy (CTE), limbic-predominant age-related TDP-43 encephalopathy (LATE).

In another embodiment, a binding molecule, particularly an antibody of the invention or antigen-binding fragment thereof as described herein specific for TDP-43 is administered to prevent, alleviate and/or treat a disease selected from: Frontotemporal dementia (FTD, such as sporadic or familial with or without motor-neuron disease (MND), with progranulin (GRN) mutation, with C9orf72 mutations, with TARDBP mutation, with valosin-containing protein (VCP) mutation, linked to chromosome 9p, corticobasal degeneration, frontotemporal lobar degeneration (FTLD) with ubiquitin-positive TDP-43 inclusions (FTLD-TDP), Argyrophilic grain disease, Pick's disease, semantic variant Primary Progressive Aphasia (svPPA), behavioural variant FTD (bvFTD), nonfluent variant Primary Progressive Aphasia (nfvPPA) and the like), Amyotrophic lateral sclerosis (ALS, such as sporadic ALS, with TARDBP mutation, with angiogenin (ANG) mutation), Alexander disease (AxD), limbic-predominant age-related TDP-43 encephalopathy (LATE), Chronic Traumatic Encephalopathy (CTE), Perry syndrome, Alzheimer's disease (AD, including sporadic and familial forms of AD), Down syndrome, Familial British dementia, Polyglutamine diseases (Huntington's disease and spinocerebellar ataxia type 3 (SCA3; also known under Machado Joseph Disease)), Hippocampal sclerosis dementia and Myopathies (sporadic inclusion body myositis, Inclusion body myopathy with a mutation in the valosin-containing protein ((VCP); associated with Paget disease of bone and frontotemporal dementia), Oculo-pharyngeal muscular dystrophy with rimmed vacuoles, Myofibrillar myopathies with mutations in the myotilin (MYOT) gene or mutations in the gene coding for desmin (DES)), Traumatic Brain Injury (TBI), Dementia with Lewy Bodies (DLB) or Parkinson's disease (PD).

### DETAILED DESCRIPTION OF EMBODIMENTS OF THE INVENTION

### X. Definitions

An “antigen binding molecule,” as used herein, is any molecule that can specifically or selectively bind to an antigen, in particular TDP-43. A binding molecule may include or be an antibody or a fragment thereof. An anti-TDP-43 binding molecule is a molecule that binds to the TDP-43 protein, such as an anti-TDP-43 antibody or fragment thereof, at a specific recognition site, epitope. That is, antigen-binding molecules of the invention bind to an epitope within the amino acid sequence of SEQ ID NO: 1. The antigen-binding molecules, in particular antibodies or antigen-binding fragments thereof, provided herein recognize full-length TDP-43. Other anti-TDP-43 binding molecules may also include multivalent molecules, multi-specific molecules (e.g., diabodies), fusion molecules, aptamers, avimers, or other naturally occurring or recombinantly created molecules. Illustrative antigen-binding molecules useful in the present invention include antibody-like molecules. An antibody-like molecule is a molecule that can exhibit functions by binding to a target molecule (See, e.g., Current Opinion in Biotechnology 2006, 17:653-658; Current Opinion in Biotechnology 2007, 18:1-10; Current Opinion in Structural Biology 1997, 7:463-469; Protein Science 2006, 15:14-27), and includes, for example, DARPins (WO 2002/020565), Affibody (WO 1995/001937), Avimer (WO 2004/044011; WO 2005/040229), Adnectin (WO 2002/032925) and fynomers (WO 2013/135588).

The terms “anti-TDP-43 antibody” and “an antibody that binds to TDP-43” or simply “antibody” as used herein refer to an antibody that is capable of binding TDP-43 with sufficient affinity such that the antibody is considered for further assessment as a potential diagnostic and/or therapeutic agent in targeting TDP-43. In general, the term “antibody” is used herein in the broadest sense and encompasses various antibody structures, including but not limited to monoclonal antibodies, polyclonal antibodies, multi-specific antibodies (e.g., bispecific or biparatopic antibodies), fully-human antibodies and antibody fragments so long as they exhibit the desired antigen-binding activity. Antibodies within the present invention may also be chimeric antibodies, recombinant antibodies, antigen-binding fragments of recombinant antibodies, humanized antibodies or antibodies displayed upon the surface of a phage or displayed upon the surface of a chimeric antigen receptor (CAR) T cell.

An “antigen-binding fragment” of an antibody, or “functional fragment thereof” refers to a molecule other than an intact, or full-length, antibody that comprises a portion of an intact, or full-length, antibody and that binds (fully or partially) the antigen to which the intact, or full-length, antibody binds. Examples of antibody fragments include but are not limited to Fv, Fab, Fab′, Fab′-SH, F(ab′)2; diabodies; linear antibodies; single-chain antibody molecules (e.g. scFv); and multi-specific antibodies formed from antibody fragments. Antigen-binding fragments may also be referred to as “functional fragments” as they retain the binding function of the original antibody from which they are derived.

An “antibody that binds to an epitope” within a defined region of a protein is an antibody that requires the presence of one or more of the amino acids within that region for binding to the protein.

In certain embodiments, an “antibody that binds to an epitope” within a defined region of a protein is identified by mutation analysis, in which amino acids of the protein are mutated, and binding of the antibody to the resulting altered protein (e.g., an altered protein comprising the epitope) is determined to be at least 20% of the binding to unaltered protein. In some embodiments, an “antibody that binds to an epitope” within a defined region of a protein is identified by mutation analysis, in which amino acids of the protein are mutated, and binding of the antibody to the resulting altered protein (e.g., an altered protein comprising the epitope) is determined to be at least 30%, at least 40%, at least 50%, at least 60%, at least 70%, at least 80%, or at least 90% of the binding to unaltered protein. In certain embodiments, binding of the antibody is determined by FACS, WB or by a suitable binding assay such as ELISA.

The term “binding to” as used in the context of the present invention defines a binding (interaction) of at least two “antigen-interaction-sites” with each other. The term “antigen-interaction-site” defines, in accordance with the present invention, a motif of a polypeptide, i.e., a part of the antibody or antigen-binding fragment of the present invention, which shows the capacity of specific interaction with a specific antigen or a specific group of antigens of TDP-43. Said binding/interaction is also understood to define a “specific recognition”. The term “specifically recognizing” means in accordance with this invention that the antibody is capable of specifically interacting with and/or binding to at least two amino acids of TDP-43 as defined herein, in particular interacting with/binding to at least two amino acids within amino acids residues 304-414 of human TDP-43 (SEQ ID NO: 1), even more particularly interacting with binding to at least two amino acids within amino acids residues 304-313, 356-361, 397-407 or 396-414 of human TDP-43 (SEQ ID NO: 1).

The term “pan TDP-43 antibody” refers to an antibody that binds to misfolded aggregated TDP-43 and non-aggregated physiological TDP-43, including monomeric TDP-43, oligomeric TDP-43, post-translationally modified TDP-43 (such as phosphorylated, ubiquitinated, acetylated, sumoylated, and/or methylated), aggregated TDP-43 and truncated TDP-43.

The term “specific interaction” as used in accordance with the present invention means that the antibody or antigen-binding fragment thereof of the invention does not or does not essentially cross-react with (poly)peptides of similar structures. Accordingly, the antibody or antigen-binding fragment thereof of the invention specifically binds to/interacts with structures of TDP-43 formed by particular amino acid sequences within amino acids residues 304-414 of human TDP-43 (SEQ ID NO: 1), more particularly binds to/interacts with structures of TDP-43 formed by particular amino acid sequences within amino acids residues 304-313, 356-361, 397-407 or 396-414 of human TDP-43 (SEQ ID NO: 1).

Cross-reactivity of antigen-binding molecules, in particular a panel of antibodies or antigen-binding fragments thereof under investigation may be tested, for example, by assessing binding of said panel of antibodies or antigen-binding fragments thereof under conventional conditions (see, e.g., Harlow and Lane, Antibodies: A Laboratory Manual, Cold Spring Harbor Laboratory Press, (1988) and Using Antibodies: A Laboratory Manual, Cold Spring Harbor Laboratory Press, (1999)) to the (poly)peptide of interest as well as to a number of more or less (structurally and/or functionally) closely related (poly)peptides. Only those constructs (i.e. antibodies, antigen-binding fragments thereof and the like) that bind to the certain structure of TDP-43 as defined herein, e.g., a specific epitope or (poly)peptide/protein of TDP-43 as defined herein but do not or do not essentially bind to any of the other epitope or (poly)peptides of the same TDP-43, are considered specific for the epitope or (poly)peptide/protein of interest and selected for further studies in accordance with the method provided herein. These methods may comprise, inter alia, binding studies, blocking and competition studies with structurally and/or functionally closely related molecules. These binding studies also comprise FACS analysis, surface plasmon resonance (SPR, e.g. with BIACORE™), analytical ultracentrifugation, isothermal titration calorimetry, fluorescence anisotropy, fluorescence spectroscopy or by radiolabelled ligand binding assays.

Accordingly, specificity can be determined experimentally by methods known in the art and methods as described herein. Such methods comprise, but are not limited to Western Blots, ELISA-, RIA-, ECL-, IRMA-tests and peptide scans.

The term “monoclonal antibody” as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Monoclonal antibodies are advantageous in that they may be synthesized by a hybridoma culture, essentially uncontaminated by other immunoglobulins. The modified “monoclonal” indicates the character of the antibody as being amongst a substantially homogeneous population of antibodies, and is not to be construed as requiring production of the antibody by any particular method. As mentioned above, the monoclonal antibodies to be used in accordance with the present invention may be made by the hybridoma method described by Kohler, Nature 256 (1975), 495.

The term “polyclonal antibody” as used herein, refers to an antibody which was produced among or in the presence of one or more other, non-identical antibodies. In general, polyclonal antibodies are produced from a B-lymphocyte in the presence of several other B-lymphocytes which produced non-identical antibodies. Usually, polyclonal antibodies are obtained directly from an immunized animal.

The term “fully-human antibody” as used herein refers to an antibody which comprises human immunoglobulin protein sequences only. A fully human antibody may contain murine carbohydrate chains if produced in a mouse, in a mouse cell or in a hybridoma derived from a mouse cell. Similarly, “mouse antibody” or “murine antibody” refers to an antibody which comprises mouse/murine immunoglobulin protein sequences only. Alternatively, a “fully-human antibody” may contain rat carbohydrate chains if produced in a rat, in a rat cell, in a hybridoma derived from a rat cell. Similarly, the term “rat antibody” refers to an antibody that comprises rat immunoglobulin sequences only. Fully-human antibodies may also be produced, for example, by phage display which is a widely used screening technology which enables production and screening of fully human antibodies. Also phage antibodies can be used in context of this invention. Phage display methods are described, for example, in U.S. Pat. Nos. 5,403,484, 5,969,108 and 5,885,793. Another technology which enables development of fully-human antibodies involves a modification of mouse hybridoma technology. Mice are made transgenic to contain the human immunoglobulin locus in exchange for their own mouse genes (see, for example, U.S. Pat. No. 5,877,397).

The term “chimeric antibodies”, refers to an antibody which comprises a variable region of the present invention fused or chimerized with an antibody region (e.g., constant region) from another, human or non-human species (e.g., mouse, horse, rabbit, dog, cow, chicken).

The term antibody also relates to recombinant human antibodies, heterologous antibodies and heterohybrid antibodies. The term “recombinant (human) antibody” includes all human sequence antibodies that are prepared, expressed, created or isolated by recombinant means, such as antibodies isolated from an animal (e.g., a mouse) that is transgenic for human immunoglobulin genes; antibodies expressed using a recombinant expression vector transfected into a host cell, antibodies isolated from a recombinant, combinatorial human antibody library, or antibodies prepared, expressed, created or isolated by any other means that involves splicing of human immunoglobulin gene sequences to other DNA sequences. Such recombinant human antibodies have variable and constant regions (if present) derived from human germline immunoglobulin sequences. Such antibodies can, however, be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

A “heterologous antibody” is defined in relation to the transgenic non-human organism producing such an antibody. This term refers to an antibody having an amino acid sequence or an encoding nucleic acid sequence corresponding to that found in an organism not consisting of the transgenic non-human animal, and generally from a species other than that of the transgenic non-human animal.

The term “heterohybrid antibody” refers to an antibody having light and heavy chains of different organismal origins. For example, an antibody having a human heavy chain associated with a murine light chain is a heterohybrid antibody. Examples of heterohybrid antibodies include chimeric and humanized antibodies.

The term antibody also relates to humanized antibodies. “Humanized” forms of non-human (e.g. murine or rabbit) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Often, humanized antibodies are human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Furthermore, humanized antibody may comprise residues, which are found neither in the recipient antibody nor in the imported CDR or framework sequences. These modifications are made to further refine and optimize antibody performance. In general, the humanized antibody will comprise substantially all of at least one, and typically two variable domains, in which all or substantially all of the CDR regions correspond to those of a non-human immunoglobulin and all or substantially all of the FR regions are those of a human immunoglobulin consensus sequence. The humanized antibody may also comprise at least a portion of an immunoglobulin constant region (Fc), typically that of a human immunoglobulin. For further details, see: Jones et al., Nature 321 (1986), 522-525; Reichmann Nature 332 (1998), 323-327 and Presta Curr Op Struct Biol 2 (1992), 593-596.

A popular method for humanization of antibodies involves CDR grafting, where a functional antigen-binding site from a non-human ‘donor’ antibody is grafted onto a human ‘acceptor’ antibody. CDR grafting methods are known in the art and described, for example, in U.S. Pat. Nos. 5,225,539, 5,693,761 and 6,407,213. Another related method is the production of humanized antibodies from transgenic animals that are genetically engineered to contain one or more humanized immunoglobulin loci which are capable of undergoing gene rearrangement and gene conversion (see, for example, U.S. Pat. No. 7,129,084).

Accordingly, in the context of the present invention, the term “antibody” relates to full immunoglobulin molecules as well as to parts of such immunoglobulin molecules (i.e., “antigen-binding fragment thereof”). Furthermore, the term relates, as discussed above, to modified and/or altered antibody molecules. The term also relates to recombinantly or synthetically generated/synthesized antibodies. The term also relates to intact antibodies as well as to antibody fragments thereof, like, separated light and heavy chains, Fab, Fv, Fab′, Fab′-SH, F(ab′)2. The term antibody also comprises but is not limited to fully-human antibodies, chimeric antibodies, humanized antibodies, CDR-grafted antibodies and antibody constructs, like single chain Fvs (scFv) or antibody-fusion proteins. “Single-chain Fv” or “scFv” antibody fragments have, in the context of the invention, the V_{H} and V_{L} domains of an antibody, wherein these domains are present in a single polypeptide chain. Generally, the scFv polypeptide further comprises a polypeptide linker between the V_{H} and V_{L} domains which enables the scFv to form the desired structure for antigen binding. Techniques described for the production of single chain antibodies are described, e.g., in Plückthun in The Pharmacology of Monoclonal Antibodies, Rosenburg and Moore eds. Springer-Verlag, N.Y. (1994), 269-315.

A “Fab fragment” as used herein is comprised of one light chain and the C_{H}1 and variable regions of one heavy chain. The heavy chain of a Fab molecule cannot form a disulfide bond with another heavy chain molecule.

An “Fc” region contains two heavy chain fragments comprising the C_{H}2 and C_{H}3 domains of an antibody. The two heavy chain fragments are held together by two or more disulfide bonds and by hydrophobic interactions of the C_{H}3 domains.

A “Fab fragment” contains one light chain and a portion of one heavy chain that contains the V_{H} domain and the C_{H}1 domain and also the region between the C_{H}1 and CH₂ domains, such that an interchain disulfide bond can be formed between the two heavy chains of two Fab′ fragments to form a F(ab′)₂ molecule.

A “F(ab′)₂ fragment” contains two light chains and two heavy chains containing a portion of the constant region between the C_{H}1 and C_{H}2 domains, such that an interchain disulfide bond is formed between the two heavy chains. A F(ab′)₂ fragment thus is composed of two Fab′ fragments that are held together by a disulfide bond between the two heavy chains.

The “Fv region” comprises the variable regions from both the heavy and light chains, but lacks the constant regions.

Antibodies, antibody constructs, antibody fragments, antibody derivatives (all being Ig-derived) to be employed in accordance with the invention or their corresponding immunoglobulin chain(s) can be further modified using conventional techniques known in the art, for example, by using amino acid deletion(s), insertion(s), substitution(s), addition(s), and/or recombination(s) and/or any other modification(s) known in the art either alone or in combination. Methods for introducing such modifications in the DNA sequence underlying the amino acid sequence of an immunoglobulin chain are well known to the person skilled in the art; see, e.g., Sambrook et al., Molecular Cloning: A Laboratory Manual; Cold Spring Harbor Laboratory Press, 2^{nd} edition (1989) and 3^{rd} edition (2001). The term “Ig-derived domain” particularly relates to (poly)peptide constructs comprising at least one CDR. Fragments or derivatives of the recited Ig-derived domains define (poly)peptides which are parts of the above antibody molecules and/or which are modified by chemical/biochemical or molecular biological methods. Corresponding methods are known in the art and described inter alia in laboratory manuals (see Sambrook et al., Molecular Cloning: A Laboratory Manual; Cold Spring Harbor Laboratory Press, 2nd edition (1989) and 3rd edition (2001); Gerhardt et al., Methods for General and Molecular Bacteriology ASM Press (1994); Lefkovits, Immunology Methods Manual: The Comprehensive Sourcebook of Techniques; Academic Press (1997); Golemis, Protein-Protein Interactions: A Molecular Cloning Manual Cold Spring Harbor Laboratory Press (2002)).

The term “CDR” as employed herein relates to “complementary determining region”, which is well known in the art. The CDRs are parts of immunoglobulins that determine the specificity of said molecules and make contact with a specific ligand. The CDRs are the most variable part of the molecule and contribute to the diversity of these molecules. There are three CDR regions CDR1, CDR2 and CDR3 in each V domain. CDR-H depicts a CDR region of a variable heavy chain and CDR-L relates to a CDR region of a variable light chain. VH means the variable heavy chain and VL means the variable light chain. The CDR regions of an Ig-derived region may be determined as described in Kabat “Sequences of Proteins of Immunological Interest”, 5th edit. NIH Publication no. 91-3242 U.S. Department of Health and Human Services (1991). CDR sequences provided herein are defined according to Kabat. However, it will be understood by the skilled person that the invention is intended to encompass binding molecules in which the CDR sequences are defined according to any useful identification/numbering scheme. For example, Chothia (Canonical structures for the hypervariable regions of immunoglobulins. Chothia C, Lesk A M. J Mol Biol. 1987 Aug. 20; 196(4):901-17), IMGT (IMGT, the international ImMunoGeneTics database. Giudicelli V, Chaume D, Bodmer J, Müller W, Busin C, Marsh S, Bontrop R, Marc L, Malik A, Lefranc M P. Nucleic Acids Res. 1997 Jan. 1; 25(1):206-11 and Unique database numbering system for immunogenetic analysis. Lefranc M P. Immunol Today. 1997 November; 18(11):509), MacCallum (MacCallum R M, Martin A C, Thornton J M, J Mol Biol. 1996 Oct. 11; 262(5):732-45) and Martin (Abhinandan K R, Martin A C R. Analysis and improvements to Kabat and structurally correct numbering of antibody variable domains. Mol Immunol. (2008) 45:3832-9. 10.1016/j.molimm.2008.05.022) numbering schemes may be adopted in order to define the CDRs.

Accordingly, in the context of the present invention, the antibody molecule described herein above is selected from the group consisting of a full antibody (immunoglobulin, like an IgG1, an IgG2, an IgA1, an IgGA2, an IgG3, an IgG4, an IgA, an IgM, an IgD or an IgE), F(ab)-, Fab′-SH-, Fv-, Fab′-, F(ab′)2-fragment, a chimeric antibody, a CDR-grafted antibody, a fully human antibody, a bivalent antibody-construct, an antibody-fusion protein, a synthetic antibody, bivalent single chain antibody, a trivalent single chain antibody and a multivalent single chain antibody.

“Humanization approaches” are well known in the art and in particular described for antibody molecules, e.g. Ig-derived molecules. The term “humanized” refers to humanized forms of non-human (e.g., murine) antibodies or fragments thereof (such as Fv, Fab, Fab′, F(ab′), scFvs, or other antigen-binding partial sequences of antibodies) which contain some portion of the sequence derived from non-human antibody. Humanized antibodies include human immunoglobulins in which residues from a complementary determining region (CDR) of the human immunoglobulin are replaced by residues from a CDR of a non-human species such as mouse, rat or rabbit having the desired binding specificity, affinity and capacity. In general, the humanized antibody will comprise substantially all of at least one, and generally two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin; see, inter alia, Jones et al., Nature 321 (1986), 522-525, Presta, Curr. Op. Struct. Biol. 2 (1992), 593-596. Methods for humanizing non-human antibodies are well known in the art. Generally, a humanized antibody has one or more amino acids introduced into it from a source which is non-human still retain the original binding activity of the antibody. Methods for humanization of antibodies/antibody molecules are further detailed in Jones et al., Nature 321 (1986), 522-525; Reichmann et al., Nature 332 (1988), 323-327; and Verhoeyen et al., Science 239 (1988), 1534-1536. Specific examples of humanized antibodies, e.g. antibodies directed against EpCAM, are known in the art (see e.g. LoBuglio, Proceedings of the American Society of Clinical Oncology Abstract (1997), 1562 and Khor, Proceedings of the American Society of Clinical Oncology Abstract (1997), 847).

Accordingly, in the context of this invention, antibody molecules or antigen-binding fragments thereof are provided, which can be humanized and can successfully be employed in pharmaceutical compositions.

It may be understood by a person skilled in the art that the epitopes may be comprised in the TDP-43 protein, but may also be comprised in a degradation product thereof or may be a chemically synthesized peptide. The amino acid positions are only indicated to demonstrate the position of the corresponding amino acid sequence in the sequence of the TDP-43 protein. The invention encompasses all peptides comprising the epitope. The peptide may be a part of a polypeptide of more than 100 amino acids in length or may be a small peptide of less than 100, preferably less than 50, more preferably less than 25 amino acids, even more preferably less than 16 amino acids. The amino acids of such peptide may be natural amino acids or nonnatural amino acids (e.g., beta-amino acids, gamma-amino acids, D-amino acids) or a combination thereof. Further, the present invention may encompass the respective retro-inverso peptides of the epitopes. The peptide may be unbound or bound. It may be bound, e.g., to a small molecule (e.g., a drug or a fluorophor), to a high-molecular weight polymer (e.g., polyethylene glycol (PEG), polyethylene imine (PEI), hydroxypropylmethacrylate (HPMA), etc.) or to a protein, a fatty acid, a sugar moiety or may be inserted in a membrane.

In order to test whether an antibody in question and the antibody of the present invention recognize the same epitope, the following competition study may be carried out: Vero cells infected with 3 MOI (multiplicity of infection) are incubated after 20 h with varying concentrations of the antibody in question as the competitor for 1 hour. In a second incubation step, the antibody of the present invention is applied in a constant concentration of 100 nM and its binding is flow-cytometrically detected using a fluorescence-labelled antibody directed against the constant domains of the antibody of the invention. Binding that conducts anti-proportional (inversely proportional) to the concentration of the antibody in question is indicative that both antibodies recognize the same epitope. However, many other assays are known in the art which may be used.

The present invention also relates to the production of specific antibodies against native polypeptides and recombinant polypeptides of TDP-43. This production is based, for example, on the immunization of animals, like mice. However, also other animals for the production of antibody/antisera are envisaged within the present invention. For example, monoclonal and polyclonal antibodies can be produced by rabbit, mice, goats, donkeys and the like. The polynucleotide encoding a correspondingly chosen polypeptide of TDP-43 can be subcloned into an appropriate vector, wherein the recombinant polypeptide is to be expressed in an organism capable of expression, for example in bacteria. Thus, the expressed recombinant protein can be intra-peritoneally injected into mice and the resulting specific antibody can be, for example, obtained from the mice serum being provided by intra-cardiac blood puncture. The present invention also envisages the production of specific antibodies against native polypeptides and recombinant polypeptides by using a DNA/RNA vaccine strategy as exemplified in the appended examples. DNA vaccine strategies are well-known in the art and encompass liposome-mediated delivery, by gene gun or jet injection and intramuscular or intradermal injection. Thus, antibodies directed against a polypeptide or a protein or an epitope of TDP-43, in particular the epitope of the antibodies provided herein, can be obtained by directly immunizing the animal by directly injecting intramuscularly the vector expressing the desired polypeptide or a protein or an epitope of TDP-43, in particular the epitope of the antibodies of the invention, which lies within amino acid residues 304-414 of SEQ ID NO:1, more particularly which lies within amino acid residues 304-313, 356-361, 397-407 or 396-414 of SEQ ID NO:1. The amount of obtained specific antibody can be quantified using an ELISA, which is also described herein below. Further methods for the production of antibodies are well known in the art, see, e.g. Harlow and Lane, “Antibodies, A Laboratory Manual”, CSH Press, Cold Spring Harbor, 1988.

Thus, under designated assay conditions, the specified antibodies and the corresponding epitope of TDP-43 bind to one another and do not bind in a significant amount to other components present in a sample. Specific binding to a target analyte under such conditions may require a binding moiety that is selected for its specificity for a particular target analyte. A variety of immunoassay formats may be used to select antibodies specifically reactive with a particular antigen. For example, solid-phase ELISA immunoassays are routinely used to select monoclonal antibodies specifically immunoreactive with an analyte. See Shepherd and Dean (2000), Monoclonal Antibodies: A Practical Approach, Oxford University Press and/or Howard and Bethell, for a description of immunoassay formats and conditions that can be used to determine specific immunoreactivity. Typically, a specific or selective reaction will be at least twice background signal to noise and more typically more than 10 to 100 times greater than background. The person skilled in the art is in a position to provide for and generate specific binding molecules directed against the novel polypeptides. For specific binding-assays it can be readily employed to avoid undesired cross-reactivity, for example polyclonal antibodies can easily be purified and selected by known methods (see Shepherd and Dean, loc. cit.).

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In certain embodiments, antibody variants having one or more amino acid substitutions are provided. Sites of interest for substitutional mutagenesis include the CDRs and FRs. Conservative substitutions are shown in Table 1 under the heading of “preferred substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved Antibody-Dependent Cellular Cytotoxicity (ADCC) or Complement-Dependent Cytotoxicity (CDC).

TABLE 1

| | | Preferred |
|---|---|---|
| Original Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gin;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more CDR residues are mutated and the variant antibodies displayed on phage and screened for a particular biological activity (e.g. binding affinity).

Alterations (e.g., substitutions) may be made in CDRs, e.g., to improve antibody affinity. Such alterations may be made in CDR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al., in Methods in Molecular Biology 178:1-37 (O'Brien et al., ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modelling. CDR-H3 and CDR-L3 in particular are often targeted.

In certain embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. Such alterations may be outside of CDR “hotspots” or SDRs. In certain embodiments of the variant VH and VL sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244: 1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex is used to identify contact points between the antibody and antigen. Such contact residues and neighbouring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody. In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al., TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties. In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn297 (e. g. complex, hybrid and high mannose structures) as measured by MALDI-TOF mass spectrometry, as described in WO2008/077546, for example. Asn297 refers to the asparagine residue located at about position 297 in the Fc region (Eu numbering of Fc region residues; see Edelman, G. M. et al., Proc. Natl. Acad. USA, 63, 78-85 (1969)); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose deficient” antibody variants include: US2003/0157108; WO2000/61739; WO2001/29246; US2003/0115614; US2002/0164328; US2004/0093621; US2004/0132140; US2004/0110704; US 2004/0110282; US2004/0109865; WO2003/085119; WO2003/084570; WO2005/035586; WO2005/035778; WO2005/053742; WO2002/031140; Okazaki et al., J. Mol. Biol. 336:1239-1249 (2004); Yamane-Ohnuki et al., Biotech. Bioeng. 87: 614 (2004). Examples of cell lines capable of producing defucosylated antibodies include Lec13 CHO cells deficient in protein fucosylation (Ripka et al., Arch. Biochem. Biophys. 249:533-545 (1986); US Pat Appl No US 2003/0157108 A1, Presta, L; and WO2004/056312 A1, Adams et al., especially at Example 11), and knockout cell lines, such as alpha-1,6-fucosyltransferase gene, FUT8, knockout CHO cells (see, e.g., Yamane-Ohnuki et al., Bioteeh. Bioeng. 87: 614 (2004); Kanda, Y. et al., Bioteehnol. Bioeng., 94(4):680-688 (2006); and WO2003/085 107).

Antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the antibody provided herein binds to pathological TDP-43 and forms an immune complex that may be cleared by antibody-dependent cellular phagocytosis (ADCP), which as a result potentiates TDP-43 clearance. ADCP is mediated by the interaction of antibody Fc fragment with Fc receptors, such as Fc gamma receptors, expressed at the surface of innate immune cells, such as microglia or dendritic cells. Fc mediated functions may be modulated to achieve the desired effect by modifying the Fc portion of antibodies.

In certain embodiments, the invention contemplates an antibody variant that possesses some but not all effector functions, which make it a desirable candidate for applications in which the half-life of the antibody in vivo is important yet certain effector functions (such as complement activation and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes and microglia express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al., Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)).

Alternatively, non-radioactive assays methods may be employed (see, for example, ACTI™ non radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells.

Alternatively, or additionally, ADCC activity of the molecule of interest may be assessed in vivo, e.g., in an animal model such as that disclosed in Clynes et al., Proc. Nat'l Acad. sci. USA 95:652-656 (1998).

C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 234, 235, 238, 265, 269, 270, 297, 327 and 329 (U.S. Pat. No. 6,737,056). Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001)). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581) or the so-called “DANG” Fc mutant with substitution of residues 265 to alanine and 297 to glycine. Alternatively, antibodies with reduced effector function include those with substitution of one or more of Fc region residues 234, 235 and 329, so-called “PG-LALA” Fc mutant with substitution of residues 234 and 235 to alanine and 329 to glycine (Lo, M. et al., Journal of Biochemistry, 292, 3900-3908). Other known mutations at position 234, 235 and 321, the so-called TM mutant containing mutations L234F/L235E/P331S in the CH2 domain, can be used (Oganesyan et al. Acta Cryst. D64, 700-704. (2008)). Antibodies from the human IgG4 isotype include mutations S228P/L235E to stabilize the hinge and to reduce FgR binding (Schlothauer et al, PEDS, 29 (10):457-466). Numbering of the constant domain is according to the EU numbering system.

Other Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature 322:738-40 (1988); U.S. Pat. Nos. 5,648,260; 5,624,821.

In certain embodiments, the Fc region is mutated to increase its affinity to FcRn at pH 6.0 and consequently extend the antibody half-life. Antibodies with enhanced affinity to FcRn include those with substitution of one or more of Fc region residues 252, 253, 254, 256, 428, 434, including the so called YTE mutation with substitution M252Y/S254T/T256E (Dall' Acqua et al, J Immunol. 169:5171-5180 (2002)) or LS mutation M428L/N434S (Zalevsky et al, Nat Biotechnol. 28(2): 157-159 (2010)).

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

In certain embodiments, an antibody provided herein may be further modified to contain additional nonproteinaceous moieties that are known in the art and readily available. The moieties suitable for derivatization of the antibody include but are not limited to water soluble polymers. Nonlimiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n vinyl pyrrolidone)polyethylene glycol, propropylene glycol homopolymers, prolypropylene oxide/ethylene oxide copolymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed. Antibodies may be produced using recombinant methods and compositions, e.g., as described in U.S. Pat. No. 4,816,567. In one embodiment, isolated nucleic acid encoding an anti-misfolded TDP-43 antibody described herein is provided. Such nucleic acid may encode an amino acid sequence comprising the VL and/or an amino acid sequence comprising the VH of the antibody (e.g., the Light and/or Heavy Chains of the antibody). In a further embodiment, one or more vectors (e.g., recombinant expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., YO, NSO, Sp20). In one embodiment, a method of making an anti-misfolded TDP-43 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-misfolded TDP-43 antibody, nucleic acid encoding an antibody, e.g., as described above, is isolated and inserted into one or more vectors for further cloning and/or expression in a host cell or a cell-free expression system. Such nucleic acid may be readily isolated and sequenced using conventional procedures (e.g., by using oligonucleotide probes that are capable of binding specifically to genes encoding the Heavy and Light Chains of the antibody).

In one embodiment, a method of making a TDP-43 binding molecule, in particular an antibody or antigen-binding fragment thereof, is provided, wherein the method comprises culturing a host cell or cell-free expression system comprising a nucleic acid encoding the TDP-43 binding molecule, as provided above, under conditions suitable for expression of the TDP-43 binding molecule, and isolating the TDP-43 binding molecule.

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Val. 248 (B. K. C. Lo, ed., Humana Press, Totowa, NJ, 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are macaque kidney CV1 line transformed by SV40 (COS-7); human embryonic kidney line (293 or 293 cells as described, e.g., in Graham et al., J. Gen Viral. 36:59 (1977)); baby hamster kidney cells (BHK); mouse Sertoli cells (TM4 cells as described, e.g., in Mather, Biol. Reprod. 23:243-251 (1980)); macaque kidney cells (CV 1); African green macaque kidney cells (VERO-76); human cervical carcinoma cells (HeLa); canine kidney cells (MDCK); buffalo rat liver cells (BRL 3A); human lung cells (WI38); human liver cells (Hep G2); mouse mammary tumor (MMT 060562); TRI cells, as described, e.g., in Mather et al., Annals N. Y Aead. Sei. 383:44-68 (1982); MRC 5 cells; and FS4 cells. Other useful mammalian host cell lines include Chinese hamster ovary (CHO) cells, including DHFR CHO cells (Urlaub et al., Proc. Natl. Acad. cii. USA 77:4216 (1980)); and myeloma cell lines such as YO, NSO and Sp2/0. For a review of certain mammalian host cell lines suitable for antibody production, see, e.g., Yazaki and Wu, Methods in Molecular Biology, Val. 248 (B. K. C. Lo, ed., Humana Press, Totowa, NJ), pp. 255-268 (2003).

Several art-known approaches exist for delivering molecules across the blood brain barrier (BBB) such as alteration of the administration route, disruption of the BBB and alteration of its permeability, nanoparticle delivery, Trojan horse approaches, receptor-mediated transport, and cell and gene therapy.

Alteration of the administration route can be achieved by direct injection into the brain (see, e.g., Papanastassiou et al., Gene Therapy 9: 398-406(2002)), implanting a delivery device in the brain (see, e.g., Gill et al., Nature Med. 9: 589-595 (2003); and Gliadel Wafers™, Guildford Pharmaceutical), and intranasal administration to bypass the BBB (Mittal et al, Drug Deliv. 21(2):75-86. (2014))

Methods of barrier disruption include, but are not limited to, ultrasound (see, e.g., U.S. Patent Publication No. 2002/0038086), osmotic pressure (e.g., by administration of hypertonic mannitol (Neuwelt, E. A., Implication of the Blood-Brain Barrier and its Manipulation, Vols 1 & 2, Plenum Press, N.Y. (1989))), permeabilization by, e.g., bradykinin or permeabilizer A-7 (see, e.g., U.S. Pat. Nos. 5,112,596, 5,268,164, 5,506,206, and 5,686,416).

Methods of altering the BBB permeability include, but are not limited to, using glucocorticoid blockers to increase permeability of the blood-brain barrier (see, e.g., U.S. Patent Application Publication Nos. 2002/0065259, 2003/0162695, and 2005/0124533); activating potassium channels (see, e.g., U.S. Patent Application Publication No. 2005/0089473), and inhibiting ABC drug transporters (see, e.g., U.S. Patent Application Publication No. 2003/0073713).

Trojan horse delivery methods of delivering the antibody or antibody fragment thereof across the blood-brain barrier include, but are not limited to, cationizing the antibodies (see, e.g., U.S. Pat. No. 5,004,697), and the use of cell-penetration peptides such as Tat peptides to gain entry into the CNS. (see, e.g. Dietz et al., J. Neurochem. 104:757-765 (2008)).

Nanoparticle delivery methods of delivering the antibody or antigen-binding fragment thereof across the blood brain barrier include, but are not limited to, encapsulating the antibody or antigen-binding fragment thereof in liposomes, or extracellular vesicles such as exosomes, that are coupled to without limitation antibody or antigen-binding fragments or alternatively peptides that bind to receptors on the vascular endothelium of the blood-brain barrier (see, e.g., U.S. Patent Application Publication No. 20020025313), and coating the antibody or antigen-binding fragment thereof in low-density lipoprotein particles (see, e.g., U.S. Patent Application Publication No. 20040204354) or apolipoprotein E (see, e.g., U.S. Patent Application Publication No. 20040131692).

Antibodies of the invention can be further modified to enhance blood brain barrier penetration. The antibody or antigen-binding fragment thereof of the invention can be fused to a polypeptide binding to a blood-brain barrier receptor. BBB receptors include, but are not limited to, transferrin receptor, insulin receptor or low-density lipoprotein receptor. The polypeptide can be a peptide, a receptor ligand, a single domain antibody (VHH), a scFv or a Fab fragment. Antibodies of the invention can also be delivered as a corresponding nucleic acid encoding for the antibody. Such nucleic acid molecule may be a part of a viral vector for targeted delivery to the blood-brain barrier or any other cell type in the CNS. A non-limiting example is a viral vector comprising a nucleic acid molecule encoding an antibody of the invention for targeted delivery to endothelial cells of the BBB, pericytes of the BBB or astrocytes. In some embodiments the endothelial cells of the BBB, pericytes of the BBB or astrocytes express and secrete the antibody into the brain parenchyma. A preferred example is a viral vector comprising a nucleic acid molecule encoding an antibody of the invention for targeted delivery in endothelial cells of the BBB, wherein the endothelial cells of the BBB express and secrete the antibody into the brain parenchyma. A viral vector may be a recombinant adeno-associated viral vector (rAAV) selected from any AAV serotype known in the art, including, without limitation, from AAV1 to AAV12 to enable the antibody or antibody fragment or antibody derivatives to be expressed intracellularly or into the brain parenchyma.

Cell therapy methods of delivering the antibody of the invention or antibody fragment or antibody derivatives across the blood brain barrier include, but are not limited to, the use of the homing capacity of Endothelial Progenitor Cells (EPCs) transfected ex vivo with vectors and the secretion and delivery of antibodies or antibody fragments to the brain by these cells, to overcome the powerful filtering activity of the BBB (see, e.g., Heller and al., J Cell Mol Med. 00:1-7 (2020)), or the use of polymeric cell implant devices loaded with genetically engineered cells, to secrete antibody or antibody fragments (see, e.g. Marroquin Belaunzaran et al. PLoS ONE 6(4): e18268 (2011)).

Pharmaceutically acceptable carriers, diluents, adjuvants and excipients are well known in the pharmaceutical art and are described, for example, in Remington's Pharmaceutical Sciences, 15th or 18th Ed. (Alfonso R. Gennaro, ed.; Mack Publishing Company, Easton, PA, 1990); Remington: the Science and Practice of Pharmacy 19th Ed. (Lippincott, Williams & Wilkins, 1995); Handbook of Pharmaceutical Excipients, 3rd Ed. (Arthur H. Kibbe, ed.; Amer. Pharmaceutical Assoc, 1999); Pharmaceutical Codex: Principles and Practice of Pharmaceutics 12th Ed. (Walter Lund ed.; Pharmaceutical Press, London, 1994); The United States Pharmacopeia: The National Formulary (United States Pharmacopeial Convention); Fiedler's “Lexikon der Hilfstoffe” 5th Ed., Edition Cantor Verlag Aulendorf 2002; “The Handbook of Pharmaceutical Excipients”, 4th Ed., American Pharmaceuticals Association, 2003; and Goodman and Gilman's: the Pharmacological Basis of Therapeutics (Louis S. Goodman and Lee E. Limbird, eds.; McGraw Hill, 1992), the disclosures of which are hereby incorporated by reference.

The carriers, diluents, adjuvants and pharmaceutical excipients can be selected with regard to the intended route of administration and standard pharmaceutical practice. These compounds must be acceptable in the sense of being not deleterious to the recipient thereof. See Remington's Pharmaceutical Sciences, 15th or 18th Ed. (Alfonso R. Gennaro, ed.; Mack Publishing Company, Easton, PA, 1990); Remington: the Science and Practice of Pharmacy 19th Ed. (Lippincott, Williams & Wilkins, 1995); Handbook of Pharmaceutical Excipients, 3rd Ed. (Arthur H. Kibbe, ed.; Amer. Pharmaceutical Assoc, 1999); Pharmaceutical Codex: Principles and Practice of Pharmaceutics 12th Ed. (Walter Lund ed.; Pharmaceutical Press, London, 1994); The United States Pharmacopeia: The National Formulary (United States Pharmacopeial Convention); Fiedler's “Lexikon der Hilfstoffe” 5th Ed., Edition Cantor Verlag Aulendorf 2002; “The Handbook of Pharmaceutical Excipients”, 4th Ed., American Pharmaceuticals Association, 2003; and Goodman and Gilman's: the Pharmacological Basis of Therapeutics (Louis S. Goodman and Lee E. Limbird, eds.; McGraw Hill, 1992), the disclosures of which are hereby incorporated by reference.

The “effective amount” of the compound which is to be administered to a subject is the dosage which according to sound medical judgement is suitable for treating, preventing or alleviating the disease, disorder or abnormality. The specific dose level and frequency of dosage can depend, e.g., upon a variety of factors including the activity of the specific compound employed, the metabolic stability and length of action of that compound, mode and time of administration. The “effective amount” of the compound which is to be administered to a subject is the dosage which according to sound medical judgement is suitable for treating, preventing or alleviating the disorder, disease, disorder or abnormality. The specific dose level and frequency of dosage can depend, e.g., upon a variety of factors including the activity of the specific compound employed, the metabolic stability and length of action of that compound, mode and time of administration, the rate of excretion, and drug combination. Patient-specific factors such as the age, body weight, general health, sex, diet, as well as the severity of the particular condition can also influence the amount which is to be administered.

The term “clearance” (also referred as “clearance value” or “CL” or “systemic clearance”) relates to the efficiency of elimination of a substance from the body. Clearance of a substance (in this case a binding molecule of the invention) is the sum of the urinary and extrarenal clearance; for substances that are eliminated by renal and extrarenal routes, plasma clearance exceeds urinary clearance. The PK properties of mAbs are a function of their large size (150 kDa), relative polarity, Fc-receptor binding and specific binding to target antigens. The primary elimination route for mAbs is cellular uptake followed by proteolytic degradation. Low clearance of mAbs from the systemic circulation enables them to be administered less frequently than peptides or small molecules, which is often more convenient for patients (Betts et al., MABs. 2018).

### XI. Invention Embodiments for TDP-43 Specific Binding Molecule

In some embodiments, a TDP-43 binding molecule, in particular a TDP-43 antibody or an antigen-binding fragment thereof is provided, comprising:
a. a VH-CDR1 comprising the amino acid sequence of SEQ ID NO: 51, a VH-CDR2 comprising the amino acid sequence of SEQ ID NO: 52 and a VH-CDR3 comprising the amino acid sequence of SEQ ID NO: 53; or
b. a VH-CDR1 comprising the amino acid sequence of SEQ ID NO: 41, a VH-CDR2 comprising the amino acid sequence of SEQ ID NO: 42 and a VH-CDR3 comprising the amino acid sequence of SEQ ID NO: 43; or
c. a VH-CDR1 comprising the amino acid sequence of SEQ ID NO: 31, a VH-CDR2 comprising the amino acid sequence of SEQ ID NO: 32 and a VH-CDR3 comprising the amino acid sequence PC (Pro-Cys); or
d. a VH-CDR1 comprising the amino acid sequence of SEQ ID NO: 21, a VH-CDR2 comprising the amino acid sequence of SEQ ID NO: 22 and a VH-CDR3 comprising the amino acid sequence of SEQ ID NO: 23; or
e. a VH-CDR1 comprising the amino acid sequence of SEQ ID NO: 11, a VH-CDR2 comprising the amino acid sequence of SEQ ID NO: 12 and a VH-CDR3 comprising the amino acid sequence of SEQ ID NO: 13.

In some embodiments, a TDP-43 binding molecule, in particular a TDP-43 antibody or an antigen-binding fragment thereof is provided, comprising:
a. a VL-CDR1 comprising the amino acid sequence of SEQ ID NO: 55, a VL-CDR2 comprising the amino acid sequence of SEQ ID NO: 56 and a VL-CDR3 comprising the amino acid sequence of SEQ ID NO: 57; or
b. a VL-CDR1 comprising the amino acid sequence of SEQ ID NO: 45, a VL-CDR2 comprising the amino acid sequence of SEQ ID NO: 46 and a VL-CDR3 comprising the amino acid sequence of SEQ ID NO: 47; or
c. a VL-CDR1 comprising the amino acid sequence of SEQ ID NO: 35, a VL-CDR2 comprising the amino acid sequence of SEQ ID NO: 36 and a VL-CDR3 comprising the amino acid sequence of SEQ ID NO: 37; or
d. a VL-CDR1 comprising the amino acid sequence of SEQ ID NO: 15, a VL-CDR2 comprising the amino acid sequence of SEQ ID NO: 26 and a VL-CDR3 comprising the amino acid sequence of SEQ ID NO: 17; or
e. a VL-CDR1 comprising the amino acid sequence of SEQ ID NO: 15, a VL-CDR2 comprising the amino acid sequence of SEQ ID NO: 16 and a VL-CDR3 comprising the amino acid sequence of SEQ ID NO: 17.

In some embodiments, a TDP-43 binding molecule, in particular a TDP-43 antibody or an antigen-binding fragment thereof is provided, comprising:
a) a Heavy Chain Variable Region (VH) comprising:
   i. a VH-CDR1 comprising the amino acid sequence of SEQ ID NO: 51, a VH-CDR2 comprising the amino acid sequence of SEQ ID NO: 52 and a VH-CDR3 comprising the amino acid sequence of SEQ ID NO: 53; or
   ii. a VH-CDR1 comprising the amino acid sequence of SEQ ID NO: 41, a VH-CDR2 comprising the amino acid sequence of SEQ ID NO: 42 and a VH-CDR3 comprising the amino acid sequence of SEQ ID NO: 43; or
   iii. a VH-CDR1 comprising the amino acid sequence of SEQ ID NO: 31, a VH-CDR2 comprising the amino acid sequence of SEQ ID NO: 32 and a VH-CDR3 comprising the amino acid sequence PC (Pro-Cys); or
   iv. a VH-CDR1 comprising the amino acid sequence of SEQ ID NO: 21, a VH-CDR2 comprising the amino acid sequence of SEQ ID NO: 22 and a VH-CDR3 comprising the amino acid sequence of SEQ ID NO: 23; or
   v. a VH-CDR1 comprising the amino acid sequence of SEQ ID NO: 11, a VH-CDR2 comprising the amino acid sequence of SEQ ID NO: 12 and a VH-CDR3 comprising the amino acid sequence of SEQ ID NO: 13; and
b) a Light Chain Variable Region (VL) comprising:
   i. a VL-CDR1 comprising the amino acid sequence of SEQ ID NO: 55, a VL-CDR2 comprising the amino acid sequence of SEQ ID NO: 56 and a VL-CDR3 comprising the amino acid sequence of SEQ ID NO: 57; or
   ii. a VL-CDR1 comprising the amino acid sequence of SEQ ID NO: 45, a VL-CDR2 comprising the amino acid sequence of SEQ ID NO: 46 and a VL-CDR3 comprising the amino acid sequence of SEQ ID NO: 47; or
   iii. a VL-CDR1 comprising the amino acid sequence of SEQ ID NO: 35, a VL-CDR2 comprising the amino acid sequence of SEQ ID NO: 36 and a VL-CDR3 comprising the amino acid sequence of SEQ ID NO: 37; or
   iv. a VL-CDR1 comprising the amino acid sequence of SEQ ID NO: 15, a VL-CDR2 comprising the amino acid sequence of SEQ ID NO: 26 and a VL-CDR3 comprising the amino acid sequence of SEQ ID NO: 17; or
   v. a VL-CDR1 comprising the amino acid sequence of SEQ ID NO: 15, a VL-CDR2 comprising the amino acid sequence of SEQ ID NO: 16 and a VL-CDR3 comprising the amino acid sequence of SEQ ID NO: 17.

In some embodiments, a TDP-43 binding molecule, in particular a TDP-43 antibody or an antigen-binding fragment thereof is provided, comprising:
a) a Heavy Chain Variable Region (VH) comprising:
   i. a VH-CDR1 comprising the amino acid sequence of SEQ ID NO: 51 or a VH-CDR1 comprising an amino acid sequence having at least 80%, 90%, 95% or 100% sequence identity to SEQ ID NO: 51; a VH-CDR2 comprising the amino acid sequence of SEQ ID NO: 52 or a VH-CDR2 comprising an amino acid sequence having at least 80%, 90%, 95% or 100% sequence identity to SEQ ID NO: 52; and a VH-CDR3 comprising the amino acid sequence of SEQ ID NO: 53 or a VH-CDR3 comprising an amino acid sequence having at least 80%, 90%, 95% or 100% sequence identity to SEQ ID NO: 53; or
   ii. a VH-CDR1 comprising the amino acid sequence of SEQ ID NO: 41 or a VH-CDR1 comprising an amino acid sequence having at least 80%, 90%, 95% or 100% sequence identity to SEQ ID NO: 41; a VH-CDR2 comprising the amino acid sequence of SEQ ID NO: 42 or a VH-CDR2 comprising an amino acid sequence having at least 80%, 90%, 95% or 100% sequence identity to SEQ ID NO: 42; and a VH-CDR3 comprising the amino acid sequence of SEQ ID NO: 43 or a VH-CDR3 comprising an amino acid sequence having at least 80%, 90%, 95% or 100% sequence identity to SEQ ID NO: 43; or
   iii. a VH-CDR1 comprising the amino acid sequence of SEQ ID NO: 31 or a VH-CDR1 comprising an amino acid sequence having at least 80%, 90%, 95% or 100% sequence identity to SEQ ID NO: 31; a VH-CDR2 comprising the amino acid sequence of SEQ ID NO: 32 or a VH-CDR2 comprising an amino acid sequence having at least 80%, 90%, 95% or 100% sequence identity to SEQ ID NO: 32; and a VH-CDR3 comprising the amino acid sequence PC (Pro-Cys); or
   iv. a VH-CDR1 comprising the amino acid sequence of SEQ ID NO: 21 or a VH-CDR1 comprising an amino acid sequence having at least 80%, 90%, 95% or 100% sequence identity to SEQ ID NO: 21; a VH-CDR2 comprising the amino acid sequence of SEQ ID NO: 22 or a VH-CDR2 comprising an amino acid sequence having at least 80%, 90%, 95% or 100% sequence identity to SEQ ID NO: 22; and a VH-CDR3 comprising the amino acid sequence of SEQ ID NO: 23 or a VH-CDR3 comprising an amino acid sequence having at least 80%, 90%, 95% or 100% sequence identity to SEQ ID NO: 23; or
   v. a VH-CDR1 comprising the amino acid sequence of SEQ ID NO: 11 or a VH-CDR1 comprising an amino acid sequence having at least 80%, 90%, 95% or 100% sequence identity to SEQ ID NO: 11; a VH-CDR2 comprising the amino acid sequence of SEQ ID NO: 12 or a VH-CDR2 comprising an amino acid sequence having at least 80%, 90%, 95% or 100% sequence identity to SEQ ID NO: 12; and a VH-CDR3 comprising the amino acid sequence of SEQ ID NO: 13 or a VH-CDR3 comprising an amino acid sequence having at least 80%, 90%, 95% or 100% sequence identity to SEQ ID NO: 13; and
b) a Light Chain Variable Region (VL) comprising:
   i. a VL-CDR1 comprising the amino acid sequence of SEQ ID NO: 55 or a VL-CDR1 comprising an amino acid sequence having at least 80%, 90%, 95% or 100% sequence identity to SEQ ID NO: 55; a VL-CDR2 comprising the amino acid sequence of SEQ ID NO: 56 or a VL-CDR2 comprising an amino acid sequence having at least 80%, 90%, 95% or 100% sequence identity to SEQ ID NO: 56; and a VL-CDR3 comprising the amino acid sequence of SEQ ID NO: 57 or a VL-CDR3 comprising an amino acid sequence having at least 80%, 90%, 95% or 100% sequence identity to SEQ ID NO: 57; or
   ii. a VL-CDR1 comprising the amino acid sequence of SEQ ID NO: 45 or a VL-CDR1 comprising an amino acid sequence having at least 80%, 90%, 95% or 100% sequence identity to SEQ ID NO: 45; a VL-CDR2 comprising the amino acid sequence of SEQ ID NO: 46 or a VL-CDR2 comprising an amino acid sequence having at least 80%, 90%, 95% or 100% sequence identity to SEQ ID NO: 46; and a VL-CDR3 comprising the amino acid sequence of SEQ ID NO: 47 or a VL-CDR3 comprising an amino acid sequence having at least 80%, 90%, 95% or 100% sequence identity to SEQ ID NO: 47; or
   iii. a VL-CDR1 comprising the amino acid sequence of SEQ ID NO: 35 or a VL-CDR1 comprising an amino acid sequence having at least 80%, 90%, 95% or 100% sequence identity to SEQ ID NO: 35; a VL-CDR2 comprising the amino acid sequence of SEQ ID NO: 36 or a VL-CDR2 comprising an amino acid sequence having at least 80%, 90%, 95% or 100% sequence identity to SEQ ID NO: 36; and a VL-CDR3 comprising the amino acid sequence of SEQ ID NO: 37 or a VL-CDR3 comprising an amino acid sequence having at least 80%, 90%, 95% or 100% sequence identity to SEQ ID NO: 37; or
   iv. a VL-CDR1 comprising the amino acid sequence of SEQ ID NO: 15 or a VL-CDR1 comprising an amino acid sequence having at least 80%, 90%, 95% or 100% sequence identity to SEQ ID NO: 15; a VL-CDR2 comprising the amino acid sequence of SEQ ID NO: 26 or a VL-CDR2 comprising an amino acid sequence having at least 80%, 90%, 95% or 100% sequence identity to SEQ ID NO: 26; and a VL-CDR3 comprising the amino acid sequence of SEQ ID NO: 17 or a VL-CDR3 comprising an amino acid sequence having at least 80%, 90%, 95% or 100% sequence identity to SEQ ID NO: 17; or
   v. a VL-CDR1 comprising the amino acid sequence of SEQ ID NO: 15 or a VL-CDR1 comprising an amino acid sequence having at least 80%, 90%, 95% or 100% sequence identity to SEQ ID NO: 15; a VL-CDR2 comprising the amino acid sequence of SEQ ID NO: 16 or a VL-CDR2 comprising an amino acid sequence having at least 80%, 90%, 95% or 100% sequence identity to SEQ ID NO: 16; and a VL-CDR3 comprising the amino acid sequence of SEQ ID NO: 17 or a VL-CDR3 comprising an amino acid sequence having at least 80%, 90%, 95% or 100% sequence identity to SEQ ID NO: 17.

In some embodiments, a TDP-43 binding molecule, in particular a TDP-43 antibody or an antigen-binding fragment thereof is provided, comprising:
a. a Heavy Chain Variable Region (VH) which comprises a VH-CDR1 comprising the amino acid sequence of SEQ ID NO: 51, a VH-CDR2 comprising the amino acid sequence of SEQ ID NO: 52 and a VH-CDR3 comprising the amino acid sequence of SEQ ID NO: 53; and a Light Chain Variable Region (VL) which comprises a VL-CDR1 comprising the amino acid sequence of SEQ ID NO: 55, a VL-CDR2 comprising the amino acid sequence of SEQ ID NO: 56 and a VL-CDR3 comprising the amino acid sequence of SEQ ID NO: 57; or
b. a Heavy Chain Variable Region (VH) which comprises a VH-CDR1 comprising the amino acid sequence of SEQ ID NO: 41, a VH-CDR2 comprising the amino acid sequence of SEQ ID NO: 42 and a VH-CDR3 comprising the amino acid sequence of SEQ ID NO: 43; and a Light Chain Variable Region (VL) which comprises a VL-CDR1 comprising the amino acid sequence of SEQ ID NO: 45, a VL-CDR2 comprising the amino acid sequence of SEQ ID NO: 46 and a VL-CDR3 comprising the amino acid sequence of SEQ ID NO: 47; or
c. a Heavy Chain Variable Region (VH) which comprises a VH-CDR1 comprising the amino acid sequence of SEQ ID NO: 31, a VH-CDR2 comprising the amino acid sequence of SEQ ID NO: 32 and a VH-CDR3 comprising the amino acid sequence PC (Pro-Cys); and a Light Chain Variable Region (VL) which comprises a VL-CDR1 comprising the amino acid sequence of SEQ ID NO: 35, a VL-CDR2 comprising the amino acid sequence of SEQ ID NO: 36 and a VL-CDR3 comprising the amino acid sequence of SEQ ID NO: 37; or
d. a Heavy Chain Variable Region (VH) which comprises a VH-CDR1 comprising the amino acid sequence of SEQ ID NO: 21, a VH-CDR2 comprising the amino acid sequence of SEQ ID NO: 22 and a VH-CDR3 comprising the amino acid sequence of SEQ ID NO: 23; and a Light Chain Variable Region (VL) which comprises a VL-CDR1 comprising the amino acid sequence of SEQ ID NO: 15, a VL-CDR2 comprising the amino acid sequence of SEQ ID NO: 26 and a VL-CDR3 comprising the amino acid sequence of SEQ ID NO: 17; or
e. a Heavy Chain Variable Region (VH) which comprises a VH-CDR1 comprising the amino acid sequence of SEQ ID NO: 11, a VH-CDR2 comprising the amino acid sequence of SEQ ID NO: 12 and a VH-CDR3 comprising the amino acid sequence of SEQ ID NO: 13; and a Light Chain Variable Region (VL) which comprises a VL-CDR1 comprising the amino acid sequence of SEQ ID NO: 15, a VL-CDR2 comprising the amino acid sequence of SEQ ID NO: 16 and a VL-CDR3 comprising the amino acid sequence of SEQ ID NO: 17.

In some embodiments, a TDP-43 binding molecule, in particular a TDP-43 antibody or an antigen-binding fragment thereof is provided, comprising:
a. a Heavy Chain Variable Region (VH) comprising the sequence of SEQ ID NO: 50 or a Heavy Chain Variable Region (VH) having at least 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 50; or
b. a Heavy Chain Variable Region (VH) comprising the sequence of SEQ ID NO: 40 or a Heavy Chain Variable Region (VH) having at least 86%, 87%, 88%, 89%, 90%, 91%, 92%, 93%, 94%, 95%, 96%, 97%, 98% or 99% sequence identity to the amino acid sequence of SEQ ID NO: 40; or
c. a Heavy Chain Variable Region (VH) comprising the sequence of SEQ ID NO: 30 or a Heavy Chain Variable Region (VH) having at least 90%, 91%, 92%, 93%, 94%, 95%, 96%, 97%, 98% or 99% sequence identity to the amino acid sequence of SEQ ID NO: 30; or
d. a Heavy Chain Variable Region (VH) comprising the sequence of SEQ ID NO: 20 or a Heavy Chain Variable Region (VH) having at least 84%, 85%, 86%, 87%, 88%, 89%, 90%, 91%, 92%, 93%, 94%, 95%, 96%, 97%, 98% or 99% sequence identity to the amino acid sequence of SEQ ID NO: 20; or
e. a Heavy Chain Variable Region (VH) comprising the sequence of SEQ ID NO: 10 or a Heavy Chain Variable Region (VH) having at least 87%, 88%, 89%, 90%, 91%, 92%, 93%, 94%, 95%, 96%, 97%, 98% or 99% sequence identity to the amino acid sequence of SEQ ID NO: 10.

In some embodiments, a TDP-43 binding molecule, in particular a TDP-43 antibody or an antigen-binding fragment thereof is provided, comprising:
a. a Light Chain Variable Region (VL) comprising the sequence of SEQ ID NO: 54 or a Light Chain Variable Region (VL) having at least 96%, 97%, 98% or 99% sequence identity to the amino acid sequence of SEQ ID NO: 54; or
b. a Light Chain Variable Region (VL) comprising the sequence of SEQ ID NO: 44 or a Light Chain Variable Region (VL) having at least 98% or 99% sequence identity to the amino acid sequence of SEQ ID NO: 44; or
c. a Light Chain Variable Region (VL) comprising the sequence of SEQ ID NO: 34 or a Light Chain Variable Region (VL) having at least 94%, 95%, 96%, 97%, 98% or 99% sequence identity to the amino acid sequence of SEQ ID NO: 34; or
d. a Light Chain Variable Region (VL) comprising the sequence of SEQ ID NO: 24 or a Light Chain Variable Region (VL) having at least 99% sequence identity to the amino acid sequence of SEQ ID NO: 24; or
e. a Light Chain Variable Region (VL) comprising the sequence of SEQ ID NO: 14.

In some embodiments, a TDP-43 binding molecule, in particular a TDP-43 antibody or an antigen-binding fragment thereof is provided, comprising:
a) a Heavy Chain Variable Region (VH) selected from:
   i. a Heavy Chain Variable Region (VH) comprising the sequence of SEQ ID NO: 50 or a Heavy Chain Variable Region (VH) having at least 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 50; or
   ii. a Heavy Chain Variable Region (VH) comprising the sequence of SEQ ID NO: 40 or a Heavy Chain Variable Region (VH) having at least 86%, 87%, 88%, 89%, 90%, 91%, 92%, 93%, 94%, 95%, 96%, 97%, 98% or 99% sequence identity to the amino acid sequence of SEQ ID NO: 40; or
   iii. a Heavy Chain Variable Region (VH) comprising the sequence of SEQ ID NO: 30 or a Heavy Chain Variable Region (VH) having at least 90%, 91%, 92%, 93%, 94%, 95%, 96%, 97%, 98% or 99% sequence identity to the amino acid sequence of SEQ ID NO: 30; or
   iv. a Heavy Chain Variable Region (VH) comprising the sequence of SEQ ID NO: 20 or a Heavy Chain Variable Region (VH) having at least 84%, 85%, 86%, 87%, 88%, 89%, 90%, 91%, 92%, 93%, 94%, 95%, 96%, 97%, 98% or 99% sequence identity to the amino acid sequence of SEQ ID NO: 20; or
   v. a Heavy Chain Variable Region (VH) comprising the sequence of SEQ ID NO: 10 or a Heavy Chain Variable Region (VH) having at least 87%, 88%, 89%, 90%, 91%, 92%, 93%, 94%, 95%, 96%, 97%, 98% or 99% sequence identity to the amino acid sequence of SEQ ID NO: 10; and
b) a Light Chain Variable Region (VL) selected from:
   i. a Light Chain Variable Region (VL) comprising the sequence of SEQ ID NO: 54 or a Light Chain Variable Region (VL) having at least 96%, 97%, 98% or 99% sequence identity to the amino acid sequence of SEQ ID NO: 54; or
   ii. a Light Chain Variable Region (VL) comprising the sequence of SEQ ID NO: 44 or a Light Chain Variable Region (VL) having at least 98% or 99% sequence identity to the amino acid sequence of SEQ ID NO: 44; or
   iii. a Light Chain Variable Region (VL) comprising the sequence of SEQ ID NO: 34 or a Light Chain Variable Region (VL) having at least 94%, 95%, 96%, 97%, 98% or 99% sequence identity to the amino acid sequence of SEQ ID NO: 34; or
   iv. a Light Chain Variable Region (VL) comprising the sequence of SEQ ID NO: 24 or a Light Chain Variable Region (VL) having at least 99% sequence identity to the amino acid sequence of SEQ ID NO: 24; or
   v. a Light Chain Variable Region (VL) comprising the sequence of SEQ ID NO: 14.

In some embodiments, a TDP-43 binding molecule, in particular a TDP-43 antibody or an antigen-binding fragment thereof is provided, comprising:
a. a Heavy Chain Variable Region (VH) comprising the sequence of SEQ ID NO: 50 or a Heavy Chain Variable Region (VH) having at least 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 50; and a Light Chain Variable Region (VL) comprising the sequence of SEQ ID NO: 54 or a Light Chain Variable Region (VL) having at least 96%, 97%, 98% or 99% sequence identity to the amino acid sequence of SEQ ID NO: 54; or
b. a Heavy Chain Variable Region (VH) comprising the sequence of SEQ ID NO: 40 or a Heavy Chain Variable Region (VH) having at least 86%, 87%, 88%, 89%, 90%, 91%, 92%, 93%, 94%, 95%, 96%, 97%, 98% or 99% sequence identity to the amino acid sequence of SEQ ID NO: 40; and a Light Chain Variable Region (VL) comprising the sequence of SEQ ID NO: 44 or a Light Chain Variable Region (VL) having at least 98% or 99% sequence identity to the amino acid sequence of SEQ ID NO: 44; or
c. a Heavy Chain Variable Region (VH) comprising the sequence of SEQ ID NO: 30 or a Heavy Chain Variable Region (VH) having at least 90%, 91%, 92%, 93%, 94%, 95%, 96%, 97%, 98% or 99% sequence identity to the amino acid sequence of SEQ ID NO: 30; and a Light Chain Variable Region (VL) comprising the sequence of SEQ ID NO: 34 or a Light Chain Variable Region (VL) having at least 94%, 95%, 96%, 97%, 98% or 99% sequence identity to the amino acid sequence of SEQ ID NO: 34; or
d. a Heavy Chain Variable Region (VH) comprising the sequence of SEQ ID NO: 20 or a Heavy Chain Variable Region (VH) having at least 84%, 85%, 86%, 87%, 88%, 89%, 90%, 91%, 92%, 93%, 94%, 95%, 96%, 97%, 98% or 99% sequence identity to the amino acid sequence of SEQ ID NO: 20; and a Light Chain Variable Region (VL) comprising the sequence of SEQ ID NO: 24 or a Light Chain Variable Region (VL) having at least 99% sequence identity to the amino acid sequence of SEQ ID NO: 24; or
e. a Heavy Chain Variable Region (VH) comprising the sequence of SEQ ID NO: 10 or a Heavy Chain Variable Region (VH) having at least 87%, 88%, 89%, 90%, 91%, 92%, 93%, 94%, 95%, 96%, 97%, 98% or 99% sequence identity to the amino acid sequence of SEQ ID NO: 10; and a Light Chain Variable Region (VL) comprising the sequence of SEQ ID NO: 14.

In some embodiments, a TDP-43 binding molecule, in particular a TDP-43 antibody or an antigen-binding fragment thereof is provided, comprising:
a. a Heavy Chain Variable Region (VH) comprising the amino acid sequence of SEQ ID NO: 50 and a Light Chain Variable Region (VL) comprising the amino acid sequence of SEQ ID NO: 54; or
b. a Heavy Chain Variable Region (VH) comprising the amino acid sequence of SEQ ID NO: 40 and a Light Chain Variable Region (VL) comprising the amino acid sequence of SEQ ID NO: 44; or
c. a Heavy Chain Variable Region (VH) comprising the amino acid sequence of SEQ ID NO: 30 and a Light Chain Variable Region (VL) comprising the amino acid sequence of SEQ ID NO: 34; or
d. a Heavy Chain Variable Region (VH) comprising the amino acid sequence of SEQ ID NO: 20 and a Light Chain Variable Region (VL) comprising the amino acid sequence of SEQ ID NO: 24; or
e. a Heavy Chain Variable Region (VH) comprising the amino acid sequence of SEQ ID NO: 10 and a Light Chain Variable Region (VL) comprising the amino acid sequence of SEQ ID NO: 14.

In some embodiments, a TDP-43 binding molecule, in particular a TDP-43 antibody or an antigen-binding fragment thereof is provided, which binds misfolded aggregated TDP-43 and non-aggregated physiological TDP-43.

In some embodiments, a TDP-43 binding molecule, in particular a TDP-43 antibody or an antigen-binding fragment thereof is provided, which binds to monomeric and/or oligomeric and/or aggregated and/or post-translationally modified and/or truncated TDP-43, preferably human TDP-43.

In some embodiments, a TDP-43 binding molecule, in particular a TDP-43 antibody or an antigen-binding fragment thereof is provided, which binds misfolded aggregated human TDP-43 and non-aggregated physiological human TDP-43.

In some embodiments, a TDP-43 binding molecule, in particular a TDP-43 antibody or an antigen-binding fragment thereof is provided, which exhibits one or more, up to all of the following characteristics:
a. inhibits the aggregation of TDP-43 protein or fragments thereof,
b. blocks TDP-43 cell-to-cell propagation;
c. disaggregates TDP-43 aggregates;
d. blocks TDP-43 seeding;
e. neutralizes seeding-competent TDP-43;
f. blocks TDP-43 spreading;
g. potentiates TDP-43 clearance; and
h. reduces phosphorylated TDP-43 level in vivo.

In some embodiments, a TDP-43 binding molecule, in particular a TDP-43 antibody or an antigen-binding fragment thereof is provided, which exhibits one or more, up to all of the following characteristics:
a. inhibits the aggregation of TDP-43 protein or fragments thereof,
b. blocks TDP-43 cell-to-cell propagation;
c. blocks TDP-43 seeding;
d. blocks TDP-43 spreading;
e. potentiates TDP-43 clearance; and
f. reduces phosphorylated TDP-43 level in vivo.

In some embodiments, a TDP-43 binding molecule, in particular a TDP-43 antibody or an antigen-binding fragment thereof is provided, which potentiates TDP-43 clearance.

In some embodiments, a TDP-43 binding molecule, in particular a TDP-43 antibody or an antigen-binding fragment thereof is provided, which reduces TDP-43 pathology in vivo.

In some embodiments, a TDP-43 binding molecule, in particular a TDP-43 antibody or an antigen-binding fragment thereof is provided, which reduces levels of misfolded aggregated TDP-43 and/or phosphorylated TDP-43 in vivo.

In some embodiments, a TDP-43 binding molecule, in particular a TDP-43 antibody or an antigen-binding fragment thereof is provided, which reduces levels of phosphorylated TDP-43 in the hippocampus.

In some embodiments, a TDP-43 binding molecule, in particular a TDP-43 antibody or an antigen-binding fragment thereof is provided, which binds to an epitope within amino acids residues 304-414 of human TDP-43 (SEQ ID NO: 1).

In some embodiments, a TDP-43 binding molecule, in particular a TDP-43 antibody or an antigen-binding fragment thereof is provided, which binds to an epitope within amino acids residues 304-313 of human TDP-43 (SEQ ID NO: 1).

In some embodiments, a TDP-43 binding molecule, in particular a TDP-43 antibody or an antigen-binding fragment thereof is provided, which binds to an epitope consisting of amino acids residues 304-313 of human TDP-43 (SEQ ID NO: 1).

In some embodiments, a TDP-43 binding molecule, in particular a TDP-43 antibody or an antigen-binding fragment thereof is provided, which binds to an epitope within amino acids residues 356-361 of human TDP-43 (SEQ ID NO: 1).

In some embodiments, a TDP-43 binding molecule, in particular a TDP-43 antibody or an antigen-binding fragment thereof is provided, which binds to an epitope consisting of amino acids residues 356-361 of human TDP-43 (SEQ ID NO: 1).

In some embodiments, a TDP-43 binding molecule, in particular a TDP-43 antibody or an antigen-binding fragment thereof is provided, which binds to an epitope within amino acids residues 397-407 of human TDP-43 (SEQ ID NO: 1).

In some embodiments, a TDP-43 binding molecule, in particular a TDP-43 antibody or an antigen-binding fragment thereof is provided, which binds to an epitope consisting of amino acids residues 397-407 of human TDP-43 (SEQ ID NO: 1).

In some embodiments, a TDP-43 binding molecule, in particular a TDP-43 antibody or an antigen-binding fragment thereof is provided, which binds to an epitope within amino acids residues 396-414 of human TDP-43 (SEQ ID NO: 1).

In some embodiments, a TDP-43 binding molecule, in particular a TDP-43 antibody or an antigen-binding fragment thereof is provided, which binds to an epitope consisting of amino acids residues 396-414 of human TDP-43 (SEQ ID NO: 1).

In some embodiments, a TDP-43 binding molecule, in particular a TDP-43 antibody or an antigen-binding fragment thereof is provided, which binds to the protease-resistant amyloid core of TDP-43. The protease-resistant amyloid core of TDP-43 is composed of amino acids 272-360 of TDP-43.

In some embodiments, the TDP-43 binding molecule is an antibody or an antigen-binding fragment thereof.

In some embodiments, a TDP-43 binding molecule, in particular a TDP-43 antibody or an antigen-binding fragment thereof is provided, which has a dissociation constant (KD) for binding soluble TDP-43 (SEQ ID NO: 1) of 1 nM or less, preferably 750 pM or less, 500 pM or less, 380 pM or less, 230 pM or less, 200 pM or less, or 110 pM or less. Reference may be made to Example 3 for further details of a suitable assay to determine KD.

In some embodiments, the TDP-43 binding molecule is an IgA, IgD, IgE, IgM, IgG1, IgG2, IgG3 or IgG4 antibody or antigen-binding fragment thereof.

In a preferred embodiment, the TDP-43 binding molecule is an IgG1 or an IgG4 antibody or antigen-binding fragment thereof.

In some embodiments, a TDP-43 binding molecule, in particular a TDP-43 antibody or an antigen-binding fragment thereof is provided, which comprises an Fc mutation, preferably the S228P mutation.

In some embodiments, a(n isolated) nucleic acid is provided, wherein the (isolated) nucleic acid encodes a TDP-43 binding molecule in particular TDP-43 antibody and fragment thereof described herein.

In some embodiments, a(n isolated) nucleic acid is provided, wherein the (isolated) nucleic acid comprises SEQ ID NO: 18 encoding a Heavy Chain Variable Region (VH) of an anti-TPD-43 antibody described herein.

In some embodiments, a(n isolated) nucleic acid is provided, wherein the (isolated) nucleic acid comprises SEQ ID NO: 19 encoding Light Chain Variable Region (VL) of an anti-TPD-43 antibody described herein.

In some embodiments, a(n isolated) nucleic acid is provided, wherein the (isolated) nucleic acid comprises SEQ ID NO: 28 encoding a Heavy Chain Variable Region (VH) of an anti-TPD-43 antibody described herein.

In some embodiments, a(n isolated) nucleic acid is provided, wherein the (isolated) nucleic acid comprises SEQ ID NO: 29 encoding Light Chain Variable Region (VL) of an anti-TPD-43 antibody described herein.

In some embodiments, a(n isolated) nucleic acid is provided, wherein the (isolated) nucleic acid comprises SEQ ID NO: 38 encoding a Heavy Chain Variable Region (VH) of an anti-TPD-43 antibody described herein.

In some embodiments, a(n isolated) nucleic acid is provided, wherein the (isolated) nucleic acid comprises SEQ ID NO: 39 encoding Light Chain Variable Region (VL) of an anti-TPD-43 antibody described herein.

In some embodiments, a(n isolated) nucleic acid is provided, wherein the (isolated) nucleic acid comprises SEQ ID NO: 48 encoding a Heavy Chain Variable Region (VH) of an anti-TPD-43 antibody described herein.

In some embodiments, a(n isolated) nucleic acid is provided, wherein the (isolated) nucleic acid comprises SEQ ID NO: 49 encoding Light Chain Variable Region (VL) of an anti-TPD-43 antibody described herein.

In some embodiments, a(n isolated) nucleic acid is provided, wherein the (isolated) nucleic acid comprises SEQ ID NO: 58 encoding a Heavy Chain Variable Region (VH) of an anti-TPD-43 antibody described herein.

In some embodiments, a(n isolated) nucleic acid is provided, wherein the (isolated) nucleic acid comprises SEQ ID NO: 59 encoding Light Chain Variable Region (VL) of an anti-TPD-43 antibody described herein.

### XII. Compositions and Methods

The invention also relates to pharmaceutical compositions comprising a TDP-43 binding molecule, particularly an antibody or an antigen-binding fragment thereof, of the invention as described herein and a pharmaceutically acceptable carrier and/or excipient and/or diluent.

In some embodiments, a pharmaceutical composition is provided, comprising an (isolated) antibody described herein and a pharmaceutically acceptable carrier.

In some embodiments, a conjugated binding molecule, in particular antibody or antigen-binding fragment thereof, is provided, comprising a binding molecule, in particular an antibody or antigen-binding fragment thereof, described herein and a conjugated molecule. Conjugates of the invention may be referred to as immunoconjugates. Any suitable conjugated molecule may be employed according to the invention. Suitable examples include, but are not limited to enzymes (e.g. alkaline phosphatase or horseradish peroxidase), avidin, streptavidin, biotin, Protein A/G, magnetic beads, fluorophores, radioactive isotopes (i.e., radioconjugates), nucleic acid molecules, detectable labels, therapeutic agents, toxins and blood brain barrier penetration moieties. Conjugation methods are well known in the art and several technologies are commercially available for conjugating antibodies to a label or other molecule, Conjugation is typically through amino acid residues contained within the binding molecules of the invention (such as lysine, histidine or cysteine). They may rely upon methods such as the NHS (Succinimidyl) ester method, isothiocyanate method, carbodiimide method and periodate method. Conjugation may be achieved through creation of fusion proteins for example. This is appropriate where the binding molecule is conjugated with another protein molecule. Thus, suitable genetic constructs may be formed that permit the expression of a fusion of the binding molecule of the invention with the label or other molecule. Conjugation may be via a suitable linker moiety to ensure suitable spatial separation of the antibody and conjugated molecule, such as detectable label. However, a linker may not be required in all instances. In some embodiments the TDP-43 specific binding molecule of the present invention is linked to a detectable label.

The invention also relates to an immunoconjugate comprising the TDP-43 binding molecule, provided herein conjugated to one or more therapeutic agents, such as chemotherapeutic agents or drugs, growth inhibitory agents, toxins (e.g., protein toxins, enzymatically active toxins of bacterial, fungal, plant, or animal origin, or fragments thereof), radioactive isotopes (i.e., a radioconjugate), blood-brain barrier penetration moieties or detectable labels. Various techniques exist for improving drug delivery across the blood-brain barrier (BBB) as discussed herein, which discussion applies mutatis mutandis. Non-invasive techniques include the so-called “Trojan horse approach” in which conjugated molecules deliver the binding molecules of the invention by binding to BBB receptors and mediating transport. Suitable molecules may comprise endogenous ligands or antibodies, in particular monoclonal antibodies, that bind specific epitopes on the BBB receptor.

In some embodiments, an immunoconjugate is provided, wherein the immunoconjugate comprises an (isolated) antibody described herein and a therapeutic agent. In some embodiments, a labelled antibody is provided, comprising an antibody described herein and a detectable label.

In some embodiments the TDP-43 specific binding molecule is part of an immunoconjugate wherein the TDP-43 specific binding molecule is covalently linked to another suitable therapeutic agent.

In some embodiments the TDP-43 specific binding molecule or the immunoconjugate comprising it is present as a composition comprising a TDP-43 specific binding molecule.

In some embodiments the TDP-43 specific binding molecule is part of pharmaceutical composition comprising a TDP-43 specific binding molecule, or an immunoconjugate wherein the TDP-43 specific binding molecule is covalently linked to another suitable therapeutic agent, or a composition comprising a TDP-43 specific binding molecule combined with a pharmaceutically acceptable carrier and/or excipient and/or diluent.

In some embodiments the immunoconjugate comprising a TDP-43 binding molecule of the invention crosses the blood brain barrier using a delivery vehicle or a blood brain barrier moiety. In some embodiments the delivery vehicle comprises a liposome or extracellular vesicle. In some embodiments the TDP-43 binding molecule is linked to the blood brain barrier moiety. In some embodiments the blood brain barrier moiety is a polypeptide or a small molecule, preferably, a peptide, a receptor ligand, a single domain antibody (VHH), a scFv or a Fab fragment. In some embodiments the blood brain barrier moiety binds a blood brain barrier receptor, which may comprise a transferrin receptor, insulin receptor or low-density lipoprotein receptor.

In some embodiments the TDP-43 specific binding molecule is part of a detection and/or diagnostic kit comprising a TDP-43 specific binding molecule, or an immunoconjugate wherein the TDP-43 specific binding molecule is covalently linked to another suitable therapeutic agent, or a composition comprising a TDP-43 specific binding molecule.

In some embodiments, a TDP-43 binding molecule described herein is used in a pairing assay comprising the steps of:
a. Incubating a sample with a capture antibody and a detect antibody;
b. Incubating the mixture obtained in step a with a reagent suitable for detection by the detect antibody;
c. Measuring the signal emitted by the detect antibody;
   wherein the capture antibody is a TDP-43 binding molecule of the invention.

In some embodiments, a TDP-43 binding molecule described herein is used in a pairing assay comprising the steps of:
a. Incubating a sample with a capture antibody and a detect antibody;
b. Incubating the mixture obtained in step a with a reagent suitable for detection by the detect antibody;
c. Measuring the signal emitted by the detect antibody;
   wherein the detect antibody is a TDP-43 binding molecule of the invention.

In some embodiments, TDP-43 binding molecules described herein are used in a pairing assay comprising the steps of:
a. Incubating a sample with a capture antibody and a detect antibody;
b. Incubating the mixture obtained in step a with a reagent suitable for detection by the detect antibody;
c. Measuring the signal emitted by the detect antibody;
   wherein the capture antibody and the detect antibody are TDP-43 binding molecules of the invention.

In some embodiments, the invention provides a method of detecting TDP-43 in a sample comprising the steps of:
a. incubating a sample with a capture antibody and a detect antibody to produce a mixture;
b. incubating the mixture obtained in step a. with a reagent suitable for detection of TDP-43 by the detect antibody; and
c. measuring the signal emitted by the detect antibody.

In one embodiment of the method of detecting TDP-43 in a sample, the capture antibody is a TDP-43 binding molecule of the invention. In another embodiment of the method of detecting TDP-43 in a sample, the detect antibody is a TDP-43 binding molecule of the invention. In a further embodiment of the method of detecting TDP-43 in a sample, the capture antibody and the detect antibody are TDP-43 binding molecules of the invention. The capture antibody and the detect antibody may be the same or different antibodies of the invention.

In some embodiments, provided is a pairing assay kit for detecting TDP-43 in a sample. The pairing assay kit comprises one or more TDP-43 binding molecules of the invention. The kit may be an Enzyme-Linked Immunosorbent Assay (ELISA) kit. The kit may be a Single Molecule Array (Simoa®) kit. The kit comprises a capture agent and/or a detect agent. The kit optionally further comprises a detection reagent. The TDP-43 binding molecule of the invention may be provided in the kit as the capture agent, such as the capture antibody and/or as the detect agent, such as a detect antibody.

In some embodiments, one or more TDP-43 binding molecule described herein is/are used in a pairing assay comprising the step of incubating a sample with a capture and a detect antibody, wherein the sample is human blood, cerebrospinal fluid (CSF), interstitial fluid (ISF) and/or urine, preferably CSF.

Kits containing the binding molecules of the invention are also provided. In particular, such kits may be useful for performing the diagnostic methods of the invention (which include classification, monitoring and therapy selection methods). Thus, a kit for diagnosis of a disease, disorder and/or abnormality associated with TDP-43, in particular associated with TDP-43 aggregates, or a TDP-43 proteinopathy, or for use in a method of the invention is provided comprising a TDP-43 specific binding molecule of the invention. Such kits may comprise all necessary components for performing the herein provided methods. Typically, each component is stored separately in a single overall packaging. Suitable additional components for inclusion in the kits are, for example, buffers, detectable dyes, laboratory equipment, reaction containers, instructions and the like. Instructions for use may be tailored to the specific method for which the kit is to be employed. Suitably labelled TDP-43 binding molecules of the invention are also provided, which may be included in such kits.

In some embodiments the TDP-43 specific binding molecule is used in an immunodiagnostic method for use in the prevention, diagnosis or treatment of a TDP-43 proteinopathy.

In some embodiments the TDP-43 specific binding molecule, or an immunoconjugate wherein the TDP-43 specific binding molecule is covalently linked to another suitable therapeutic agent, or a composition comprising a TDP-43 specific binding molecule, is administered to a subject in need thereof or is used to diagnose, prevent, alleviate or treat a disease, disorder and/or abnormality associated with TDP-43, in particular associated with TDP-43 aggregates, or TDP-43 proteinopathy including but not limited to frontotemporal dementia (FTD), amyotrophic lateral sclerosis (ALS), Alzheimer's disease (AD), Parkinson's disease (PD), Chronic Traumatic Encephalopathy (CTE), limbic-predominant age-related TDP-43 encephalopathy (LATE).

In some embodiments the TDP-43 specific binding molecule, or an immunoconjugate wherein the TDP-43 specific binding molecule is covalently linked to another suitable therapeutic agent, or a composition comprising a TDP-43 specific binding molecule, is administered to a subject in need thereof or is used in a method for diagnosing or monitoring a disease, disorder and/or abnormality associated with TDP-43, in particular associated with TDP-43 aggregates, or TDP-43 proteinopathy selected from Frontotemporal dementia (FTD, such as sporadic or familial with or without motor-neuron disease (MND), with progranulin (GRN) mutation, with C9orf72 mutations, with TARDBP mutation, with valosin-containing protein (VCP) mutation, linked to chromosome 9p, corticobasal degeneration, frontotemporal lobar degeneration (FTLD) with ubiquitin-positive TDP-43 inclusions (FTLD-TDP), Argyrophilic grain disease, Pick's disease, semantic variant Primary Progressive Aphasia (svPPA), behavioural variant FTD (bvFTD), nonfluent variant Primary Progressive Aphasia (nfvPPA) and the like), Amyotrophic lateral sclerosis (ALS, such as sporadic ALS, with TARDBP mutation, with angiogenin (ANG) mutation), Alexander disease (AxD), limbic-predominant age-related TDP-43 encephalopathy (LATE), Chronic Traumatic Encephalopathy (CTE), Perry syndrome, Alzheimer's disease (AD, including sporadic and familial forms of AD), Down syndrome, Familial British dementia, Polyglutamine diseases (Huntington's disease and spinocerebellar ataxia type 3 (SCA3; also known under Machado Joseph Disease)), Hippocampal sclerosis dementia and Myopathies (sporadic inclusion body myositis, Inclusion body myopathy with a mutation in the valosin-containing protein ((VCP); associated with Paget disease of bone and frontotemporal dementia), Oculo-pharyngeal muscular dystrophy with rimmed vacuoles, Myofibrillar myopathies with mutations in the myotilin (MYOT) gene or mutations in the gene coding for desmin (DES)), Traumatic Brain Injury (TBI), Dementia with Lewy Bodies (DLB) or Parkinson's disease (PD).

In other embodiment, the invention relates to any methods for detecting, diagnosing or monitoring a disease, disorder and/or abnormality associated with TDP-43, in particular associated with TDP-43 aggregates, or TDP-43 proteinopathy that is selected from frontotemporal dementia (FTD), amyotrophic lateral sclerosis (ALS), Alzheimer's disease (AD), Parkinson's disease (PD), Chronic Traumatic Encephalopathy (CTE), and limbic-predominant age-related TDP-43 encephalopathy (LATE).

Preferably, the disease, disorder and/or abnormality associated with TDP-43, in particular associated with TDP-43 aggregates, or TDP-43 proteinopathies selected from amyotrophic lateral sclerosis (ALS), Alzheimer's disease (AD) and Frontotemporal dementia (FTD). More preferably, the disease, disorder and/or abnormality associated with TDP-43, in particular associated with TDP-43 aggregates, or TDP-43 proteinopathy is amyotrophic lateral sclerosis (ALS). More preferably, the disease, disorder and/or abnormality associated with TDP-43, in particular associated with TDP-43 aggregates, or TDP-43 proteinopathy is Alzheimer's disease (AD). More preferably, the disease, disorder and/or abnormality associated with TDP-43, in particular associated with TDP-43 aggregates, or TDP-43 proteinopathy is Frontotemporal dementia (FTD).

In some embodiments, the TDP-43 specific binding molecule is used in a method for diagnosing presymptomatic disease or for monitoring disease progression and therapeutic efficacy, or for predicting responsiveness, or for selecting subjects which are likely to respond to the treatment with a TDP-43 specific binding molecule. Said method is preferably performed using a sample of human blood or urine. Most preferably the method involves an ELISA-based or surface adapted assay.

In some embodiments the TDP-43 specific binding molecule is used in a method wherein a TDP-43 specific binding molecule of the present invention is contacted with a sample (e.g., blood, urine, cerebrospinal fluid, interstitial fluid (ISF), or brain tissue) to detect, diagnose or monitor frontotemporal degeneration (FTD) or amyotrophic lateral sclerosis (ALS), Alzheimer's disease (AD), Chronic Traumatic Encephalopathy (CTE), Perry syndrome, limbic-predominant age-related TDP-43 encephalopathy (LATE) and/or Parkinson's disease (PD).

In some embodiments, the TDP-43 specific binding molecule is used in a method wherein a TDP-43 specific binding molecule of the present invention is contacted with a sample (e.g., blood, urine, cerebrospinal fluid, interstitial fluid (ISF), or brain tissue) to detect, diagnose or monitor a disease selected from Frontotemporal dementia (FTD, such as sporadic or familial with or without motor-neuron disease (MND), with progranulin (GRN) mutation, with C9orf72 mutations, with TARDBP mutation, with valosin-containing protein (VCP) mutation, linked to chromosome 9p, corticobasal degeneration, frontotemporal lobar degeneration (FTLD) with ubiquitin-positive TDP-43 inclusions (FTLD-TDP), Argyrophilic grain disease, Pick's disease, semantic variant Primary Progressive Aphasia (svPPA), behavioural variant FTD (bvFTD), nonfluent variant Primary Progressive Aphasia (nfvPPA) and the like), Amyotrophic lateral sclerosis (ALS, such as sporadic ALS, with TARDBP mutation, with angiogenin (ANG) mutation), Alexander disease (AxD), limbic-predominant age-related TDP-43 encephalopathy (LATE), Chronic Traumatic Encephalopathy (CTE), Perry syndrome, Alzheimer's disease (AD, including sporadic and familial forms of AD), Down syndrome, Familial British dementia, Polyglutamine diseases (Huntington's disease and spinocerebellar ataxia type 3 (SCA3; also known under Machado Joseph Disease)), Hippocampal sclerosis dementia and Myopathies (sporadic inclusion body myositis, Inclusion body myopathy with a mutation in the valosin-containing protein ((VCP); associated with Paget disease of bone and frontotemporal dementia), Oculo-pharyngeal muscular dystrophy with rimmed vacuoles, Myofibrillar myopathies with mutations in the myotilin (MYOT) gene or mutations in the gene coding for desmin (DES)), Traumatic Brain Injury (TBI), Dementia with Lewy Bodies (DLB) or Parkinson's disease (PD).

In some embodiments, the TDP-43 specific binding molecule, or an immunoconjugate wherein the TDP-43 specific binding molecule is covalently linked to another suitable therapeutic agent, or a composition comprising a TDP-43 specific binding molecule, is administered to a subject in need thereof or is used for preventing, alleviating or treating a disease, disorder and/or abnormality associated with TDP-43, in particular associated with TDP-43 aggregates, or TDP-43 proteinopathies, or frontotemporal degeneration (FTD) or amyotrophic lateral sclerosis (ALS), Alzheimer's disease (AD, including sporadic and familial forms of AD), Chronic Traumatic Encephalopathy (CTE), Perry syndrome and limbic-predominant age-related TDP-43 encephalopathy (LATE) and/or Parkinson's disease (PD).

In some embodiments, the TDP-43 specific binding molecule, or an immunoconjugate wherein the TDP-43 specific binding molecule is covalently linked to another suitable therapeutic agent, or a composition comprising a TDP-43 specific binding molecule, is administered to a subject in need thereof or is used for treating a disease selected from: Frontotemporal dementia (FTD, such as sporadic or familial with or without motor-neuron disease (MND), with progranulin (GRN) mutation, with C9orf72 mutations, with TARDBP mutation, with valosin-containing protein (VCP) mutation, linked to chromosome 9p, corticobasal degeneration, frontotemporal lobar degeneration (FTLD) with ubiquitin-positive TDP-43 inclusions (FTLD-TDP), Argyrophilic grain disease, Pick's disease, semantic variant Primary Progressive Aphasia (svPPA), behavioural variant FTD (bvFTD), nonfluent variant Primary Progressive Aphasia (nfvPPA) and the like), Amyotrophic lateral sclerosis (ALS, such as sporadic ALS, with TARDBP mutation, with angiogenin (ANG) mutation), Alexander disease (AxD), limbic-predominant age-related TDP-43 encephalopathy (LATE), Chronic Traumatic Encephalopathy (CTE), Perry syndrome, Alzheimer's disease (AD, including sporadic and familial forms of AD), Down syndrome, Familial British dementia, Polyglutamine diseases (Huntington's disease and spinocerebellar ataxia type 3 (SCA3; also known under Machado Joseph Disease)), Hippocampal sclerosis dementia and Myopathies (sporadic inclusion body myositis, Inclusion body myopathy with a mutation in the valosin-containing protein ((VCP); associated with Paget disease of bone and frontotemporal dementia), Oculo-pharyngeal muscular dystrophy with rimmed vacuoles, Myofibrillar myopathies with mutations in the myotilin (MYOT) gene or mutations in the gene coding for desmin (DES)), Traumatic Brain Injury (TBI), Dementia with Lewy Bodies (DLB) or Parkinson's disease (PD). Preferably said disease treatment helps to retain or increase mental recognition and or reduces the level of TDP-43 aggregates in the brain.

In some embodiments the TDP-43 specific binding molecule, or an immunoconjugate wherein the TDP-43 specific binding molecule is covalently linked to another suitable therapeutic agent, or a composition comprising a TDP-43 specific binding molecule, is administered to a subject in need thereof or is used for manufacturing a medicament for treating a disease, disorder and/or abnormality associated with TDP-43, in particular associated with TDP-43 aggregates, or TDP-43 proteinopathies or frontotemporal degeneration (FTD) or amyotrophic lateral sclerosis (ALS), Alzheimer's disease (AD, including sporadic and familial forms of AD), Chronic Traumatic Encephalopathy (CTE), Perry syndrome and limbic-predominant age-related TDP-43 encephalopathy (LATE), and/or Parkinson's disease (PD).

Pharmaceutical formulations of an anti-TDP-43 antibody (the preferred type of TDP-43 specific binding molecule) or immunoconjugate as described herein are prepared by mixing such antibody or immunoconjugate having the desired degree of purity with one or more optional pharmaceutically acceptable carriers and/or excipients and/or diluents (Remington's Pharmaceutical Sciences 16th edition, Osol, A. Ed. (1980)). Typically, the antibody or fragment therefor is prepared as a lyophilized formulation or aqueous solution. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases. Pharmaceutically acceptable excipients that may be used to formulate the compositions include, but are not limited to: ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene-block polymers, polyethylene glycol and lanolin. Diluents may be buffers. They may comprise a salt selected from the group consisting of phosphate, acetate, citrate, succinate and tartrate, and/or wherein the buffer comprises histidine, glycine, TRIS glycine, Tris, or mixtures thereof. It is further envisaged in the context of the present invention that the diluent is a buffer selected from the group consisting of potassium phosphate, acetic acid/sodium acetate, citric acid/sodium citrate, succinic acid/sodium succinate, tartaric acid/sodium tartrate, and histidine/histidine HCl or mixtures thereof.

Exemplary lyophilized antibody or immunoconjugate formulations are described in U.S. Pat. No. 6,267,958. Aqueous antibody or immunoconjugate formulations include those described in U.S. Pat. No. 6,171,586 and WO2006/044908, the latter formulations including a histidine-acetate buffer.

The formulation herein may also contain more than one active ingredient as necessary for the particular indication being treated, preferably those with complementary activities that do not adversely affect each other.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody or immunoconjugate, which matrices are in the form of shaped articles, e.g. films, or microcapsules. The formulations to be used for in vivo administration are generally sterile. Sterility may be readily accomplished, e.g., by filtration through sterile filtration membranes.

Any of the antigen-binding molecules, anti-TDP-43 antibodies or immunoconjugates provided herein may be used in methods, e.g., therapeutic methods.

In another aspect, an anti-TDP-43 antibody (the preferred type of TDP-43 specific binding molecule) or immunoconjugate for use as a medicament is provided. In further aspects, an anti-misfolded TDP-43 antibody (the preferred type of TDP-43 specific binding molecule) or immunoconjugate for use in a method of treatment is provided. In certain embodiments, an anti-TDP-43 antibody (the preferred type of TDP-43 specific binding molecule) or immunoconjugate for use in the prevention, diagnosis and/or treatment of a TDP-43 proteinopathy is provided. In a preferred embodiment of the invention, an anti-TDP-43 antibody (the preferred type of TDP-43 specific binding molecule) or immunoconjugate is provided for use in the prevention, diagnosis and/or treatment of a disease, disorder and/or abnormality associated with TDP-43, in particular associated with TDP-43 aggregates, or TDP-43 proteinopathy including but not limited to frontotemporal dementia (FTD), amyotrophic lateral sclerosis (ALS), Alzheimer's disease (AD), Parkinson's disease (PD), Chronic Traumatic Encephalopathy (CTE), and/or limbic-predominant age-related TDP-43 encephalopathy (LATE).

In a further aspect, the invention provides for the use of an anti-TDP-43 antibody (the preferred type of TDP-43 specific binding molecule) or immunoconjugate in the manufacture or preparation of a medicament. In one such embodiment, the method further comprises administering to the individual an effective amount of at least one additional therapeutic agent, e.g., as described below.

A “subject” or an “individual” according to any of the embodiments may be an animal, a mammal, preferably a human.

In a further aspect, the invention provides pharmaceutical formulations comprising any of the anti-TDP-43 antibodies (the preferred type of TDP-43 specific binding molecule) or immunoconjugate provided herein, e.g., for use in any of the therapeutic methods. In one embodiment, a pharmaceutical formulation comprises any of the anti-TDP-43 antibodies (the preferred type of TDP-43 specific binding molecule) or immunoconjugates provided herein and a pharmaceutically acceptable carrier and/or excipients and/or diluents (as discussed elsewhere herein). In another embodiment, a pharmaceutical formulation comprises any of the anti-TDP-43 antibodies (the preferred type of TDP-43 specific binding molecule) or immunoconjugates provided herein and at least one additional therapeutic agent, e.g., as described below.

Antibodies or immunoconjugates of the invention can be used either alone or in combination with other agents in a therapy. For instance, an antibody (the preferred type of TDP-43 specific binding molecule) or immunoconjugate of the invention may be co-administered with at least one additional therapeutic agent targeting alpha-synuclein, BACE1, tau, beta-amyloid, TDP-43 or a neuroinflammation protein.

For instance, an antibody (the preferred type of TDP-43 specific binding molecule) or immunoconjugate of the invention may be co-administered with at least one additional therapeutic agent which is selected from, but not limited to, neurological drugs, anti-amyloid beta antibodies, anti-tau antibodies, tau aggregation inhibitors (including small molecules), beta-amyloid aggregation inhibitors (including small molecules), anti-BACE1 antibodies, BACE1 inhibitors, anti-alpha-synuclein inhibitors, anti-alpha-synuclein antibodies and neuroinflammation inhibitors.

Such combination therapies noted above encompass combined administration (where two or more therapeutic agents are included in the same or separate formulations), and separate administration, in which case, administration of the antibody (the preferred type of TDP-43 specific binding molecule) or immunoconjugate of the invention can occur prior to, simultaneously, and/or following, administration of the additional therapeutic agent and/or adjuvant. Antibodies (the preferred type of TDP-43 specific binding molecule) or immunoconjugates of the invention can also be used in combination with radiation therapy.

An antibody (the preferred type of TDP-43 specific binding molecule) or immunoconjugate of the invention (and any additional therapeutic agent) can be administered by any suitable means, including parenteral, intrapulmonary, and intranasal, and, if desired for local treatment, intralesional, intrauterine or intravesical administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies (the preferred type of TDP-43 specific binding molecule) or immunoconjugates of the invention would be formulated, dosed, and administered in a fashion consistent with good medical practice. Factors for consideration in this context include the particular disease, disorder and/or abnormality associated with TDP-43, in particular associated with TDP-43 aggregates, or TDP-43 proteinopathy being treated, the particular mammal being treated, the clinical condition of the individual subject, the cause of the disease, a disorder and/or abnormality associated with TDP-43, in particular associated with TDP-43 aggregates, or TDP-43 proteinopathy, the site of delivery of the agent, the method of administration, the scheduling of administration, and other factors known to medical practitioners. The antibody or immunoconjugate need not be, but is optionally formulated with one or more agents currently used to prevent or treat the disease, disorder and/or abnormality associated with TDP-43, in particular associated with TDP-43 aggregates, or TDP-43 proteinopathy in question. The effective amount of such other agents depends on the amount of antibody or immunoconjugate present in the formulation, the type of disease, disorder and/or abnormality associated with TDP-43, in particular associated with TDP-43 aggregates or TDP-43 proteinopathy or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody (the preferred type of TDP-43 specific binding molecule) or immunoconjugate of the invention (when used alone or in combination with one or more other additional therapeutic agents) will depend on the type of disease to be treated, the type of antibody or immunoconjugate, the severity and course of the disease, whether the antibody or immunoconjugate is administered for preventive or therapeutic purposes, previous therapy, the subject's clinical history and response to the antibody or immunoconjugate, and the discretion of the attending physician. The antibody (the preferred type of TDP-43 specific binding molecule) or immunoconjugate is suitably administered to the subject at one time or over a series of treatments. Depending on the type and severity of the disease, about 1 μg/kg to 15 mg/kg (e.g. 0.1 mg/kg-10 mg/kg) of antibody (the preferred type of TDP-43 specific binding molecule) or immunoconjugate can be an initial candidate dosage for administration to the subject, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody or immunoconjugate would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the subject. Such doses may be administered intermittently, e.g. every week or every three weeks (e.g. such that the subject receives from about two to about twenty, or e.g. about six doses of the antibody). An initial higher loading dose, followed by one or more lower doses may be administered. However, other dosage regimens may be useful. The progress of this therapy is easily monitored by conventional techniques and assays.

It is understood that any of the above formulations or therapeutic methods may be carried out using both an immunoconjugate of the invention and an anti-TDP-43 antibody (the preferred type of TDP-43 specific binding molecule).

In another aspect of the invention, an article of manufacture containing materials useful for the treatment, prevention and/or diagnosis of the diseases, disorders or abnormalities associated with TDP-43, in particular associated with TDP-43 aggregates, or TDP-43 proteinopathy, described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the disease, disorder and/or abnormality associated with TDP-43, in particular associated with TDP-43 aggregates, or TDP-43 proteinopathy, and may have a sterile access port (for example the container may be an intravenous solution bag or a vial having a stopper pierceable by a hypodermic injection needle). At least one active agent in the composition is an antibody or immunoconjugate of the invention. The label or package insert indicates that the composition is used for treating the condition of choice.

Moreover, the article of manufacture may comprise (a) a first container with a composition contained therein, wherein the composition comprises an antibody (the preferred type of TDP-43 specific binding molecule) or immunoconjugate of the invention; and (b) a second container with a composition contained therein, wherein the composition comprises a further therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically acceptable buffer, such as bacteriostatic water for injection (BWFI), phosphate-buffered saline, Ringer's solution or dextrose solution. It may further include other materials desirable from a commercial and user standpoint, including other buffers, diluents, filters, needles, and syringes.

In a further embodiment, the invention relates to a method of retaining or increasing cognitive memory capacity, movement and language function or preventing and/or slowing decline of cognitive memory capacity, movement and language function in a subject, comprising administering the binding molecule of the invention, the immunoconjugate of the invention, the composition of the invention or the pharmaceutical composition of the invention.

In a further embodiment, the invention relates to a method of reducing the level of TDP-43, comprising administering the binding molecule of the invention, the immunoconjugate of the invention, the composition of the invention or the pharmaceutical composition of the invention.

The methods of the invention may comprise administering at least one additional therapy, preferably wherein the additional therapy is selected from, but not limited to, antibodies or small molecules targeting alpha-synuclein, BACE1, tau, beta-amyloid, TDP-43 or a neuroinflammation protein, in particular neurological drugs, anti-beta-amyloid antibodies, anti-tau antibodies, tau aggregation inhibitors, beta-amyloid aggregation inhibitors, anti-BACE1 antibodies, BACE1 inhibitors, anti-alpha-synuclein antibodies and neuroinflammation inhibitors.

The invention furthermore relates to a method of detecting TDP-43, comprising contacting a sample with the binding molecule of the invention, preferably an antibody of the invention wherein the sample is a brain sample, a cerebrospinal fluid sample, an interstitial fluid (ISF) sample, urine sample or a blood sample.

In further embodiment, the invention relates to a method of detecting and/or measuring the level of TDP-43, comprising contacting a sample with the binding molecule of the invention, preferably an antibody of the invention, using Single Molecule Array (SIMOA®) technology, wherein the sample is a human blood sample, a cerebrospinal fluid sample (CSF), an interstitial fluid (ISF) sample or a urine sample, preferably a CSF sample.

As described herein, the binding molecules (preferably antibodies) of the invention target (i.e. bind to) particular domains or fragments of TDP-43. For example, the ACI-7071-810H12-Ab1 antibody binds to the protease-resistant amyloid core of TDP-43. Thus, the methods may be based on detecting and/or measuring the level of the specific domain or fragments of TDP-43. For example, the methods may be based on detecting and/or measuring the level of the protease-resistant amyloid core of TDP-43 in a C terminal fragment. This may provide an indication of disease or disease status, given the observation that disease-specific proteolytic cleavage exposing this amyloid-core further enhances its seeding activity, which is important for templated aggregation.

In certain embodiments, the TDP-43 binding molecule, in particular TDP-43 antibody and fragment thereof as provided herein has a dissociation constant (KD) of ≤1 M, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M), in particular with respect to binding TDP-43, in particular soluble TDP-43. For example, the TDP-43 binding molecules of the invention may have a KD for binding soluble full-length TDP-43 of 2 nM or less, in specific embodiments 1 nM or less, in more specific embodiments a KD for soluble full-length TDP-43 of 750 pM or less, 500 pM or less, 380 pM or less, 230 pM or less, 200 pM or less, or 110 pM or less. This is demonstrated for TDP-43 binding molecules of the invention in Example 3 with reference to Table 4. In one embodiment, binding affinity to full length (FL) TDP-43 may be evaluated by determining the dissociation constants (KD) using surface plasmon resonance (SPR; Biacore 8K, GE Healthcare Life Sciences). Reference may be made to Example 3 for a detailed description of suitable SPR methods that may be employed.

In an embodiment, the TDP-43 binding molecule, in particular TDP-43 antibody and fragment thereof may reduce the level of pathological TDP-43 in the brain and ameliorate/inhibit/reduce the formation of TDP-43 pathology in vivo.

In a further embodiment, the TDP-43 binding molecule, in particular TDP-43 antibody and fragment thereof may reduce the level of phosphorylated TDP-43 in the brain and ameliorate/inhibit/reduce the formation of TDP-43 pathology in vivo.

In yet a further embodiment, the TDP-43 binding molecule, in particular TDP-43 antibody and fragment thereof may reduce the level of phosphorylated TDP-43 in the hippocampus and ameliorate/inhibit/reduce the formation of TDP-43 pathology in vivo.

TDP-43 binding molecules of the invention, in particular the antibodies or antigen-binding fragments thereof, typically bind TDP-43 with high affinity. For example, they may demonstrate an EC50 value of 200 pM or less, 40 pM or less, 20 pM or less, or 10 pM or less as determined by Luminex Assay. Reference may be made to Example 2 for further details of a suitable assay.

TDP-43 binding molecules of the invention, in particular the antibodies or antigen-binding fragments thereof, may have a half-life in mice of at least 10 days or at least 16 days. Reference can be made to Example 9 for further details of a suitable way to measure half-life in mice.

### BRIEF DESCRIPTION OF THE FIGURES

FIG. 1. Graphical representation of phosphorylated TDP-43 (pTDP-43) quantification from the ipsilateral hippocampus (FIG. 1a) or contralateral hippocampus (FIG. 1b) of a TDP-43 proteinopathy mouse model treated with ACI-7071-806H5-Ab1 (B), ACI-7071-810H12-Ab1 (C) or a mAb negative control antibody (D). (E) Non-inoculated mice. (F) monogenic CamK2a mice lacking the human TDP-43 transgene (WT-tTA). Mean±SD.

FIG. 2. Graphical representation of antibody plasma exposure at study endpoint following 13 weekly ACI-7071-810H12-Ab1 i.p. administrations each at 60 mg/kg in CamKIIa-hTDP-43NLSm mice. Mean±SD.

FIG. 3. Graphical representation of the linear relationship between pTDP-43 level and ACI-7071-810H12-Ab1 plasma concentration at study endpoint in ipsilateral (black line and dots) and contralateral (light grey line and triangles) sides.

FIG. 4. Immunoblot using (A) ACI-7071-810H12-Ab1, (B) an anti-total TDP-43 antibody or (C) an anti-pTDP-43 (S409/S410) antibody to detect TDP-43 in sarkosyl-insoluble brain extracts prepared from frontal cortex of cases with FTLD-TDP type A before (−) or after (+) limited proteolysis. Top arrows indicate the expected molecular weight for full length TDP-43 and pTDP-43. Bottom arrow indicates the expected molecular weight of protease-resistant fragment. Brace indicates expected molecular weight for TDP-43 C-terminal fragments (CTFs). Bottom part of the blot provides an enhanced contrast image of the immunoblot at the expected molecular weight of the protease-resistant fragment (around 8-15 kDa).

### EXAMPLES

### Example 1. Preparation of a TDP-43 Vaccine Composition

The liposome-based vaccines were prepared according to the protocols published in WO2012/055933. Vaccines containing full length TDP-43 (FL TDP-43) protein as antigen (Table 2, SEQ ID NO: 1) were used for antibody generation.

TABLE 2
TDP-43 protein and peptide antigen description

| SEQ ID NO | Definition | Amino acid sequence (1-letter code) |
|---|---|---|
| SEQ ID NO: 1 | Q13148 (UniProt) | MSEYIRVTEDENDEPIEIPSEDDGTVLLSTVTAQFP |
| | TADBP_HUMAN | GACGLRYRNPVSQCMRGVRLVEGILHAPDAGWG |
| | TAR DNA- | NLVYVVNYPKDNKRKMDETDASSAVKVKRAVQ |
| | binding | KTSDLIVLGLPWKTTEQDLKEYFSTFGEVLMVQV |
| | protein 43 | KKDLKTGHSKGFGFVRFTEYETQVKVMSQRHMI |
| | a.a. 1-414 | DGRWCDCKLPNSKQSQDEPLRSRKVFVGRCTED |
| | | MTEDELREFFSQYGDVMDVFIPKPFRAFAFVTFA |
| | | DDQIAQSLCGEDLIIKGISVHISNAEPKHNSNRQ |
| | | LERSGRFGGNPGGFGNQGGFGNSRGGGAGLGNNQG |
| | | SNMGGGMNFGAFSINPAMMAAAQAALQSSWGM |
| | | MGMLASQQNQSGPSGNNQNQGNMQREPNQAFG |
| | | SGNNSYSGSNSGAAIGWGSASNAGSGSGFNGGFG |
| | | SSMDSKSSGWGM |

### Example 2. Generation of Anti-TDP-43 Antibodies

### A. Mouse Immunization

Female C57BL/6JOlaHsd (C57BL/6) and BALB/c OlaHsd (BALB/c) wild-type mice (Harlan, USA) were received at 9 weeks of age. Vaccinations started at 10 weeks. Mice were vaccinated with full-length TDP-43 protein presented on the surface of liposomes in the presence of Monophosphoryl Hexa-acyl Lipid A, 3-Deacyl (Synthetic) (3D-(6-acyl) PHAD®) as adjuvant. Mice were vaccinated by 200 μl subcutaneous injection (s.c.) on days 0, 4, 8, 21, 35, and 70. Mice were bled and heparinized plasma prepared 7 days before immunization (pre-immune plasma) and on days 15, 28, 42, 77 and 136 after first immunization. Mice used for myeloma fusion were additionally vaccinated with three daily booster injections of TDP-43 protein per i.p. injection without adjuvant. Vaccine response was measured in mouse plasma. Binding of plasma derived antibodies from immunized mice to immobilized recombinant full-length (FL) TDP-43 indicated high titers for antibodies against TDP-43.

### B. Generation of Hybridomas and Selection for Subcloning

Mice were euthanized and fusion with myeloma cells was performed using splenocytes from four individual mice. Screening for antibodies from the successfully fused hybridoma cell lines were performed as follows. Diluted (1:32) cell culture supernatants were analyzed using Luminex bead-based multiplex assay (Luminex, The Netherlands). Luminex beads were conjugated to FL TDP-43 and with capturing IgGs with anti-mouse IgG-Fc antibodies specific for the IgG1, IgG2a, IgG2b, IgG2c, and IgG3 subclasses (Jackson Immunoresearch, USA). Binding to beads conjugated to FL TDP-43 identified 386 hits derived from mice immunized with the FL TDP-43 liposomal vaccine.

Viable hybridomas were grown using serum-containing selection media. Clones with preferential binding to TDP-43 inclusions in human FTD brain and clones binding to C-terminus of TDP-43 were selected for further subcloning. Following limiting dilution, the clonal hybridomas were grown in low immunoglobulin containing medium and stable colonies were selected for antibody screening and selection. Antibodies shown in Table 3 were identified from this screen.

TABLE 3
EC50 values determined by Luminex Assay

| | | Heavy | Light | EC50 (pM) |
|---|---|---|---|---|
| Hybridoma | | chain | chain | TDP-43 |
| Clone | Antibody Name | Isotype | isotype | protein |
| 704H9A12 | ACI-7071-704H9-Ab1 | IgG2c | λ | 20 |
| 707A6F11 | ACI-7071-707A6-Ab1 | IgG2b | λ | <10 |
| 801H1E2 | ACI-7071-801H1-Ab1 | IgG2a | κ | >100 |
| 806H5B7 | ACI-7071-806H5-Ab1 | IgG1 | κ | <10 |
| 810H12B4 | ACI-7071-810H12-Ab1 | IgG1 | κ | 40 |

### Example 3. Characterization of Antibodies by Surface Plasmon Resonance (SPR)

Measurements were performed on a Biacore 8K instrument (GE Healthcare Life Sciences) by immobilizing soluble TDP-43 on a CM5 Series S sensor chip (GE Healthcare, BR-1005-30).

### KD Determination for Soluble TDP-43 by SPR

The instrument was primed with running buffer PBS-P+ and flow cells (Fc) 1 and 2 of channels 1-8 were activated with a fresh solution of EDC/NHS (Amine Coupling Kit, 1:1 ratio of both reagents, GE Healthcare Life Sciences, BR-1006-33) at 10 μL/min for 420 sec. Soluble TDP-43 (Selvita) was diluted in sodium acetate pH 4.5 to a final concentration of 5 μg/mL and injected for 80 sec with a flow rate of 10 μL/min on Fc 2. All flow cells were quenched with 1 M ethanol amine (GE Healthcare Life Sciences, BR-1006-33), at 10 μL/min for 420 sec. Immobilization levels after ethanolamine quenching were around 370 RU on all eight channels. Prior to analysis, three Startup cycles were run. Increasing mAbs concentrations were injected in single-cycle kinetics ranging from 1.2 to 100 nM, prepared from a 3-fold serial dilution in running buffer, with a contact time of 300 sec and a dissociation time of 3600 sec at a flow rate of 30 L/min. Each cycle was followed by one regeneration using 10 mM Glycine-HCl pH 1.7 with a contact time of 30 sec at 10 L/min, followed by a stabilization period of 300 sec. Results obtained from single-cycle kinetics were double-referenced using the blank Fc 1 and buffer cycles and evaluated using Biacore 8K evaluation software using the 1:1 kinetic fit model with RI and global Rmax. The following kinetic parameters were obtained (Table 4): association rate constant (ka), dissociation rate constant (kd), affinity constant (KD).

TABLE 4
ka, kd, KD value of antibodies on soluble TDP-43

TDP-43

| | ka | kd | KD |
|---|---|---|---|
| mAb | (1/Ms) | (1/s) | (nM) |
| ACI-7071-704H9-Ab1 | 7.15E+04 | 1.61E−05 | 0.23 |
| ACI-7071-707A6-Ab1 | 8.59E+04 | 9.67E−06 | 0.11 |
| ACI-7071-801H1-Ab1 | 4.80E+04 | 3.50E−05 | 0.75 |
| ACI-7071-806H5-Ab1 | 4.07E+05 | 8.04E−05 | 0.2 |
| ACI-7071-810H12-Ab1 | 6.08E+04 | 2.32E−05 | 0.38 |

### Example 4: Determination of Binding Regions

### A. Epitope Mapping Using Peptide Array for ACI-7071-704H9-Ab1, ACI-7071-707A6-Ab1, ACI-7071-801H1-Ab1 and ACI-7071-810H12-Ab1

Epitope mapping was confirmed using a custom-made peptide array library (Pepscan, Netherlands). Briefly, arrays of overlapping linear peptides covering the entire TDP-43 were used to define epitopes.

### Synthesis of Peptides

To reconstruct epitopes of the target molecule, a library of peptide-based mimics was synthesized using Fmoc-based solid-phase peptide synthesis. An amino functionalized polypropylene support was obtained by grafting with a proprietary hydrophilic polymer formulation, followed by reaction with t-butyloxycarbonyl-hexamethylenediamine (BocHMDA) using dicyclohexylcarbodiimide (DCC) with N-hydroxybenzotriazole (HOBt) and subsequent cleavage of the Boc-groups using trifluoroacetic acid (TFA). Standard Fmoc-peptide synthesis was used to synthesize peptides on the amino-functionalized solid support by custom modified JANUS liquid handling stations (Perkin Elmer).

### ELISA Screening

The binding of antibody to each of the synthesized peptides was tested in a pepscan-based ELISA. The peptide arrays were incubated with primary antibody solution (overnight at 4° C.). After washing, the peptide arrays were incubated with a 1/1000 dilution of an appropriate antibody peroxidase conjugate for one hour at 25° C. After washing, the peroxidase substrate 2,2′-azino-di-3-ethylbenzthiazoline sulfonate (ABTS) and 20 μl/ml of 3 percent H2O2 were added. After one hour, the colour development was measured. The colour development was quantified with a charge coupled device (CCD) camera and an image processing system. Determined epitopes are provided in Table 5.

TABLE 5
Epitopes for tested antibodies

| Antibody | Epitopes, a.a. of SEQ ID NO: 1 |
|---|---|
| ACI-7071-704H9-Ab1 | 356-361 |
| ACI-7071-707A6-Ab1 | 356-361 |
| ACI-7071-801H1-Ab1 | 397-407 |
| ACI-7071-810H12-Ab1 | 304-313 |

### B. Determination of Binding Regions Using an ELISA Assay for ACI-7071-806H5-Ab1

ACI-7071-806H5-Ab1 was screened by an ELISA assay to determine binding region using a library of peptides biotinylated on N-terminus. Peptide sequences are provided in Table 6. 96-well streptavidin-coated ELISA plate was incubated with 5 μg/mL of biotinylated peptides. The plate was washed 4 times with 0.05% Tween-20/PBS and then blocked with 1% bovine serum albumin (BSA) in 0.05% Tween-20/PBS for 1 hour at 37° C. The antibody purified from hybridoma supernatant was then added at 1 μg/ml and incubated for 2 hours at 37° C. after which the plate was washed. An AP-conjugated anti-mouse IgG secondary antibody (Jackson ImmunoResearch Laboratories, United Kingdom) was added at 1/1000 dilution in 0.05% Tween-20/PBS for 1 hour at 37° C. After the final wash, the plate was incubated with pNPP (Sigma-Aldrich, Switzerland), an AP substrate solution, and read at 405 nm using an ELISA plate reader (Tecan). Determined binding region is provided in Table 7. The tested antibody was found to bind to the following peptide: TP-52, TP-95, TP-96, TP-102 corresponding respectively to regions 402-414, 396-414, 396-414 (pS403/404) and 402-414 (pS409/410).

TABLE 6
Peptides used for determination of binding regions by ELISA

| Peptide number | A.a. position in SEQ ID NO: 1 |
|---|---|
| TP-52 | 402-414 |
| TP-95 | 396-414 |
| TP-96 | 396-414 (with phosphorylated S403/404) |
| TP-102 | 402-414 (with phosphorylated S409/410) |

TABLE 7
Binding regions for tested antibodies

| Antibody | Binding regions, a.a. of SEQ ID NO: 1 |
|---|---|
| ACI-7071-806H5-Ab1 | 396-414 |

### Example 5: Detection of TDP-43 in Brain Tissues from FTD/ALS Subjects by Immunohistochemistry

Target engagement was evaluated in immunohistochemistry experiments on tissues from FTD subject brains. Human FTD brain tissues were obtained from UCSF Neurodegenerative Disease Brain Bank. All material has been collected from donors from whom a written informed consent for brain autopsy and the use of the material and clinical information for research purposes has been obtained by the brain bank. Immunohistochemistry was performed on 10 m thick frozen sections using fluorescently labelled secondary antibody for detection. The following antibody was used as control: rat monoclonal anti-phospho TDP-43 p409/410 antibody (Biolegend, 829901) to detect phosphorylated TDP-43 and secondary antibody without primary antibody (No 1° Ab) to detect non-specific background.

All antibodies of the present invention bound to non-aggregated physiological nuclear TDP-43, as well as aggregated TDP-43. The detailed evaluation of binding characteristics is summarized in Table 8.

TABLE 8
Detection of TDP-43 in brain tissues from FTLD-TDP subjects

| | IHC detection of | IHC detection of nuclear |
|---|---|---|
| Antibody Name | aggregated TDP-43 | non-aggregated TDP-43 |
| ACI-7071-704H9-Ab1 | +++ | +++ |
| ACI-7071-707A6-Ab1 | +++ | +++ |
| ACI-7071-801H1-Ab1 | ++ | +++ |
| ACI-7071-806H5-Ab1 | +++ | + |
| ACI-7071-810H12-Ab1 | +++ | ++ |

NA data not available;
− absent;
+/− not clear;
+ weak;
++ medium;
+++ abundant

### Example 6: In Vitro Functionality in Recombinant TDP-43 Aggregation Assay

To evaluate functionality of antibodies in vitro, the ability of antibodies to inhibit TDP-43 aggregation was tested. FL TDP-43 was fused at C-terminus to maltose binding protein (MBP) which was separated by a Tobacco Etch Virus (TEV) protease cleavage site and produced recombinantly. Aggregation of 2.5 pM TDP-43-TEV-MBP fusion protein in 30 mM Tris, 150 mM NaCl, pH 7.4 in the presence of 2.5 pM of each anti-TDP-43 antibody or negative control mAb that does not bind to TDP-43 was induced by addition of TEV protease (AcTEV, Invitrogen) and absorbance was monitored in a μclear 96 well plate (Greiner) at 600 nm over 1.5 h. For evaluation, end points were normalized to negative control mAb and the percentage of aggregated TDP-43 was calculated for each antibody. All antibodies significantly inhibited TDP-43 aggregation by more than 97% compared to the negative control mAb (Table 9). p value <0.0001 was obtained for each mAb when compared with a control mAb, statistical analysis was performed using one way ANOVA followed by Dunnett's multiple comparison test.

TABLE 9
Inhibition of TDP-43 aggregation

| Antibody Name | % inhibition of TDP-43 aggregation |
|---|---|
| ACI-7071-704H9-Ab1 | 99.3 |
| ACI-7071-707A6-Ab1 | 98.1 |
| ACI-7071-801H1-Ab1 | 100 |
| ACI-7071-806H5-Ab1 | 100 |
| ACI-7071-810H12-Ab1 | 97.4 |

### Example 7: In Vitro Functionality in Immunodepletion of FTLD-TDP Brain-Derived TDP-43 Seeds

Sarkosyl-insoluble brain fractions (Sarko-spin) were prepared following a published protocol (Laferriere et al., 2019). Immunodepletion was performed using Dynabeads® protein G magnetic beads. For a single reaction, 20 μL of beads were used with 3 g of antibody for 10 g of sarkosyl-insoluble brain extracts (total protein). Before addition of antibodies, beads were first rinsed twice with 500 μL PBS-0.05% Tween®-20 and then washed with 100 μL of PBS-0.05% Tween®-20. 100 μL of antibodies at 30 g/mL in PBS-0.05% Tween®-20 was used to re-suspend the beads. The beads/antibody reaction was incubated for 30 minutes at room temperature under constant rotation and shaking (HulaMixer™ Sample Mixer 15920D, Thermofisher). The beads/antibody complexes were rinsed twice with PBS-0.05% Tween®-20 and once with PBS before addition of sarkosyl-insoluble brain extracts. The pool of FTLD-TDP type A brain extract was diluted to 100 g/mL. The beads/antibody complexes were resuspended using 100 μL of extract and incubated 30 minutes at room temperature under constant rotation and shaking. Supernatants were collected as immuno-depleted fractions using the magnetic support and characterized by western blotting. ACI-7071-806H5-Ab1 was able to efficiently immunodeplete TDP-43 seed from FTLD-TDP brain extracts (Table 10).

TABLE 10
Immunodepletion of TDP-43 seed in patient brain extracts

| Antibody Name | Immunodepletion |
|---|---|
| ACI-7071-806H5-Ab1 | +++ |

NA data not available;
− absent;
+/− not clear;
+ weak;
++ medium;
+++ abundant

### Example 8: In Vitro Functionality in Uptake of TDP-43 Aggregates by Microglia

For mouse primary microglia preparation, cortices isolated from CD1 mice (Charles River, France) at post-natal day 5 (P5) were dissociated enzymatically and mechanically as described in Neural Tissue Dissociation Kits (P) (Miltenyi, 130-092-628). From the cell suspension obtained, microglia were purified using CD11b/c microbeads as per manufacturer's instructions (Miltenyi, 130-093-634). Microglia were plated at a density of 3×105 cells per well onto 60 inner wells of a 96-well tissue culture plates (Falcon, 353219) and maintained in complete growth medium adapted from (5). Growth medium was composed of DMEM/F12 (Gibco, 31331-093) supplemented with 2.5% heat inactivated FBS, 1% PS, 200 ng/mL Tumor growth factor β2 (TGF-β2; Peprotech, 100-35B), 100 ng/mL Interleukin-34 (IL-34; R&D Systems), 5 g/ml N-acetyl cysteine (Sigma, A9165), 5 g/ml insulin (Sigma, I6634), 100 g/mL apotransferrin (Sigma, T1147), 100 ng/mL sodium selenite (Sigma, S-5261) and ovine wool cholesterol (1.5 μg/mL, Avanti Polar Lipids). During experiment basal medium was used: DMEM/F12 (Gibco, 31331-093) supplemented with 1% Penicillin/Streptomycin, 5 μg/ml N-acetyl cysteine (Sigma, A9165), 5 μg/ml insulin (Sigma, I6634), 100 μg/mL apotransferrin (Sigma, T1147) and 100 ng/mL sodium selenite (Sigma, S-5261).

Microglia were plated at 30,000 cells per well in growth medium and incubated for 48 h. Immunocomplexes were prepared in basal medium at 2× final concentration by mixing pHrodo™ labelled TDP-43 aggregates and ACI-7071-806H5-Ab1, ACI-7071-810H12-Ab1 or a negative control mAb in a dilution plate (Eppendorf 96-well sterile), and incubating overnight at 4° C. The dilution plate was equilibrated at RT while the cells were washed three times with basal medium. After the final wash, 100 μL of basal medium remained on the cells to which 100 μL of pHrodo™ labelled TDP-43 aggregates from dilution plate was added. Cells were immediately placed inside the Incucyte for live imaging of phase contrast (to delineate microglia) and green fluorescence (to quantify labelled TDP-43 inside microglia) for 24 h. In presence of the two mAbs ACI-7071-806H5-Ab1 and ACI-7071-810H12-Ab1, the uptake of TDP-43 aggregates by microglia was significantly increased (Table 11). p value <0.001 was obtained for each mAb when compared with a negative control mAb, statistical analysis was performed using one way ANOVA followed by Tukey's multiple comparison.

TABLE 11
Percentage increase in uptake of immunecomplexes by microglia

| | Microglial uptake (fold increase |
|---|---|
| Antibody Name | vs. negative control mAb) |
| ACI-7071-806H5-Ab1 | 3.4 |
| ACI-7071-810H12-Ab1 | 4.5 |

### Example 9: Pharmacokinetics in Mice

Nine female mice (C57BL/6 strain, age 7-11 weeks) for each tested antibodies were used in this study. Animals were purchased from Lingchang/Vital River Laboratory Animal Co., Ltd. Free plasma concentrations of ACI-7071-806H5-Ab1 and ACI-7071-810H12-Ab1 were determined after single intraperitoneal (i.p.) administrations of 60 mg/kg of ACI-7071-806H5-Ab1 and ACI-7071-810H12-Ab1, respectively. Plasma samples were collected from the tail vein from 3 mice per time-point after dose administration at the following time-points: 0.25 h, 1 h, 8 h, 24 h, 72 h and day 7, day 10, day 14, day 21 and day 28, except at time-points 72 h where six mice were sampled. Table 12 shows the half-life obtained for the two antibodies.

TABLE 12
Half-life of antibodies in mice

| Antibody Name | Half-life (days) (mouse, i.p.) |
|---|---|
| ACI-7071-806H5-Ab1 | 10.3 |
| ACI-7071-810H12-Ab1 | 16.8 |

Both antibodies presented good and similar PK parameters which make them desirable candidates for applications in which the in vivo half-life of the antibody is important, for example therapeutic use in humans.

### Example 10: In Vivo Functional Efficacy

The objective of this study was to evaluate the therapeutic effects of intraperitoneally (i.p.) delivered antibodies in a mouse model of TDP-43 proteinopathies.

### Methods

### In-Life Phase

Double transgenic CamKIIa-hTDP43NLSm animals were generated by crossing hemizygous females (JAX Stock #14650: B6;C3-Tg(tetO-TARDBP*)4Vle/J) with hemizygous males (JAX Stock #007004: B6. Cg-Tg(CamKIIa-tTA)1Mmay/DboJ). Breeders and mice were kept on 200 mg/kg doxycycline (DOX) diet until 12.5±2 weeks of age. At 13.5±2 weeks of age, CamKIIa-hTDP43NLSm mice were deeply anesthetized with buprenorphine at 1 mg/kg and immobilized in a stereotaxic frame. Sarkosyl-insoluble extracts from brain of FTLD-TDP cases were sonicated prior to injection in the dorsal hippocampus. Each injection site (needle introduced in the left hemisphere following Bregma coordinate: −2.0 mm anterior and left 1.3 mm from midline; three dorsal hippocampus location with initial depth of −1.95 mm below the dura and then partially withdrawing the needle to −1.55 mm for the second injection and again to −1.15 mm for the final injection) received 1 μl of sarkosyl-insoluble extract at a rate of 0.3 μl/min with a 4 min rest period following injection. One day later, weekly intraperitoneal injection of mAbs (60 mg/kg) started, for 13 consecutive weeks. Three different antibodies were tested: ACI-7071-806H5-Ab1, ACI-7071-810H12-Ab1 and a negative control mAb.

### Terminal Tissue Collection was Done 3 Months Post Injection.

Tissue mounting, sectioning, immunofluorescence staining and quantification Frozen tissue blocks were sectioned at 20 m thickness per section. Staining was performed on a Leica BOND-RX. Coronal sections from 4 levels covering hippocampus were processed for immunolabeling. For the triple pTDP43/NeuN/Iba1 immunofluorescence (IF) staining, the slides initially underwent a fixation/permeabilization step in Methanol/Acetone (1:1) for 10 min and washed in PBS. Then epitopes were retrieved in Leica ER1 buffer pH6 (AR9640) for 10 min at 100° C., followed by an incubation with Protein Block (PowerVision IHC/ISH Super Blocking, Leica, Ref. PV6122). Slides were then incubated with primary antibodies in two steps, first with phospho-TDP43 Ab (Biolegend, Ref. 829901, Rat Ab, 1/500), followed by a mix of NeuN (Millipore, Ref. MAB377(CH), Mouse Ab, 1/500) and Iba1 (Wako, Ref. 019-19741, Rabbit Ab, 1/1000). Next, secondary antibodies were incubated in two steps, firstly with a mix of three antibodies: anti-mouse-Cy3 (Jackson, Goat Ab, 1/200), anti-Rabbit-Alexa488 (Jackson, Goat Ab, 1/200) and anti-Rat-biotin (Jackson, Goat Ab, 1/250), and secondly with Streptavidin-Cy5 (Jackson, 1/300). Finally, slides were incubated with DAPI (1/300). All antibodies were diluted in BOND antibody diluent (Cat no. AR9352) and slides were mounted in antifade and cover slipped.

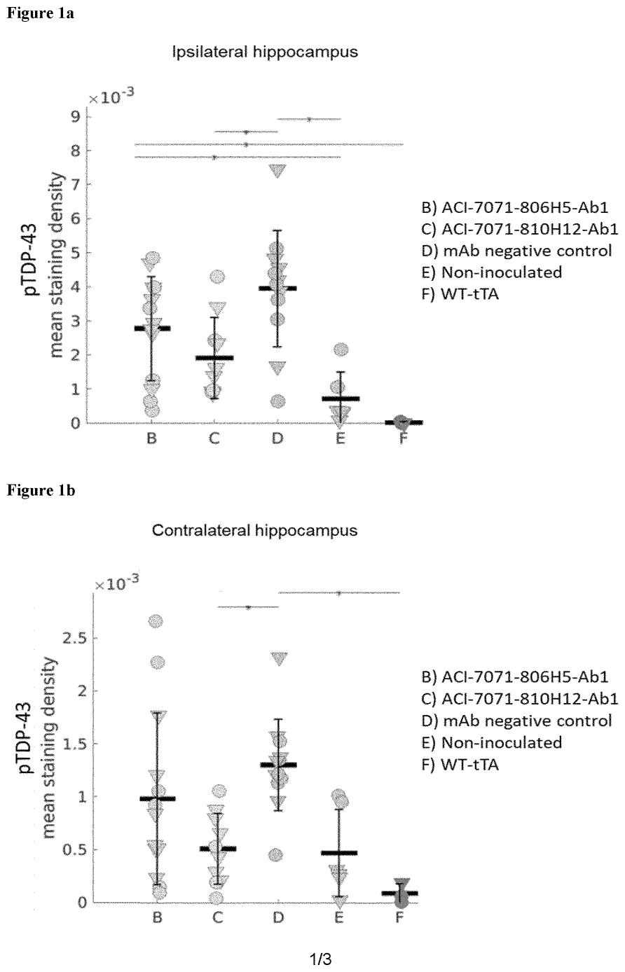

The IF slides were digitized using an Axio Scan.Z1 digital whole slide scanner (Carl Zeiss, Canada). The images underwent quality control (QC) review, and final images were transferred to the Biospective server for image processing and analysis. ROIs were delimited using a U-Net convolutional neural network trained on a dataset of manually painted tissue sections. The ROIs then underwent visual QC review, and were manually adjusted, if needed. Quantification of IHC staining (presented as mean staining density in FIG. 1) was performed on each of the digitized IHC slides using Biospective's PERMITS™ software. Additionally, double colocalization of pTDP-43 and NeuN, and triple colocalization of pTDP-43, NeuN, and Iba1 were calculated from the segmented images. The IHC analysis and quantification was performed in a blinded manner with respect to cohort. Any potential outliers for technical reasons were removed before un-blinding the data. Data are represented as mean±standard deviation.

### Statistical Analysis

Statistical analyses were performed in MATLAB. The data were first assessed for normality with a normal probability plot, followed by assessment of homogeneity of variance, if appropriate. Treatment groups were compared using one-way ANOVA (anova1) with Tukey's honest significant difference for post-hoc comparisons (multcompare) or, for non-normally distributed clinical data, using Kruskal-Wallis (kruskalwallis). For body weight measures, a two-way mixed ANOVA was also used to investigate any interaction between group and timepoint. As additional, exploratory measures, direct t-test or Mann-Whitney comparisons were made between two groups. (p value <0.05 is shown by asterisk *).

### Results

Inoculation of brain extracts in double transgenic mice (CamKIIa-hTDP43NLSm) resulted in pTDP-43 pathology in both ipsilateral (same brain hemisphere where the brain extracts were injected) and contralateral (brain hemisphere opposite to the brain extracts injection site) brain hippocampus when compared to the non-inoculated mice (E in FIG. 1) or the (WT-tTA) monogenic CamK2a mice lacking the human TDP-43 transgene (F in FIG. 1).

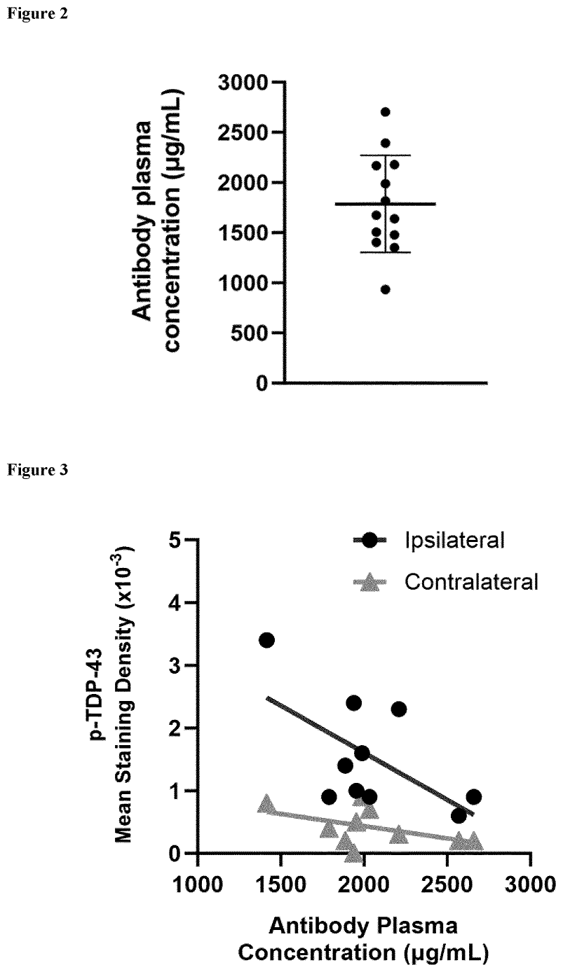

Thirteen weekly intraperitoneal administration of ACI-7071-810H12-Ab1 resulted in steady state antibody plasma levels measured at study end point of 1787 μg/mL (FIG. 2). The theoretical concentration in the brain was calculated assuming a 0.1% blood to brain penetration, resulting in 1787 ng/mL. The comparison of the ACI-7071-810H12-Ab1 KD (0.38 pM or 57 ng/ml) with the theoretical brain concentration (1787 ng/mL≈30 fold higher than the KD) highlights a favourable ratio for ACI-7071-810H12-Ab1 therapeutic use. ACI-7071-810H12-Ab1 treated mice demonstrated a statistically significant reduction in the pTDP-43 (C in FIG. 1) as compared with the negative control mAb (D in FIG. 1) in both ipsilateral and contralateral brain hippocampus. Moreover, linear regression analysis showed a trend of negative relationship between ACI-7071-810H12-Ab1 antibody exposure and the amount of pTDP-43 pathology (in both ipsilateral and contralateral side), confirming an exposure-response relationship (Ipsilateral: r²=0.37 and p=0.06; Contra: r²=0.23 and p=0.17; FIG. 3). ACI-7071-806H5-Ab1 demonstrated a trend in the reduction of pTDP-43 pathology in both ipsilateral and contralateral sides (B in FIG. 1). These data demonstrate that the tested monoclonal antibody can effectively capture the extracellular TDP-43 responsible for spreading of TDP-43 pathology in this mouse model.

### Example 11 Antibody Sequencing

Clonal hybridoma cell lysates were used for gene sequencing of the variable region. Mouse hybridomas were harvested and lysed using a lysis buffer containing guanidinium salts to deactivate RNases. cDNA was obtained by reverse transcription of total mRNA. DNA fragment coding for antibody variable region were amplified by RACE-PCR (Takara Bio, cat #634839) using specific primer annealing in the antibody constant region. PCR products were gel purified and cloned into shuttle vector for Sanger sequencing. Sequencing was carried out in both directions to provide overlap at both ends. The sequences were analyzed using multiple sequence alignment (Clustal tool) and annotated using the algorithm of Kabat as described in Kabat et al., Sequences of Proteins of Immunological Interest, 91-3242 (1991). Nucleotide sequences of the Heavy Chain (VH) and Light Chain (VL) Variable Regions are shown in Table 13. Translated protein sequences for selected Heavy (VH) and Light (VL) Chain Variable Regions, and their complementarity-determining regions (CDRs) are shown in Table 14.

### Example 12. ACI-7071-810H12-Ab1 Binding to Protease-Resistant Amyloid Core of TDP-43

ACI-7071-810H12-Ab1 antibody binding to the protease-resistant amyloid core of TDP-43 was assessed.

An immunoblot was performed on sarkosyl-insoluble brain extracts from FTLD-TDP type A patients (prepared as described previously; Laferriere et al., 2019) with and without pronase treatment. Briefly, sarkosyl-insoluble samples were treated with 0.4 mg/mL pronase (Sigma, 10165921001) for 1 h at 21° C., then centrifuged at 20,000 g for 30 min at 4° C. Supernatant was discarded and pellet was resuspended in PBS by sonication with a sonicator probe (Q-Sonica) 30 times at amplitude 30.

Sarkosyl-insoluble extracts were mixed with 4× Sample Loading buffer and 0.1 mM Dithiothreitol (DTT) and boiled for 10 min at 95° C. The samples were loaded on a 4-12% Bis-Tris gel and migrated at 100 Volt (V) for 90 min. The proteins were transferred onto a nitrocellulose membrane using the iBlot 2 system (20 milliampere (mA), 7 min). The membrane was blocked for 1 h at RT under agitation in LI-COR® blocking buffer. Primary antibodies (ACI-7071-810H12-Ab1, TDP-43 (Proteintech, 60019-2-Ig) or pTDP-43 (Biolegend, 829901)) were diluted to 1:1000 in PBS-0.1% Tween-20/LI-COR® blocking buffer (1:1) and incubated on the membrane overnight at 4° C. under agitation. The membrane was washed 3 times in PBS-0.1% Tween-20 under agitation. Secondary antibodies Donkey anti-mouse IRDye680CW and Donkey anti-rat IRDye800CW (LI-COR®) were diluted 1:10,000 in the same buffer as the primary antibodies and the membranes were incubated for 1 h at RT under constant agitation. The membranes were scanned after 3 washes in PBS with 0.1% Tween-20 using LI-COR® Odyssey imager.

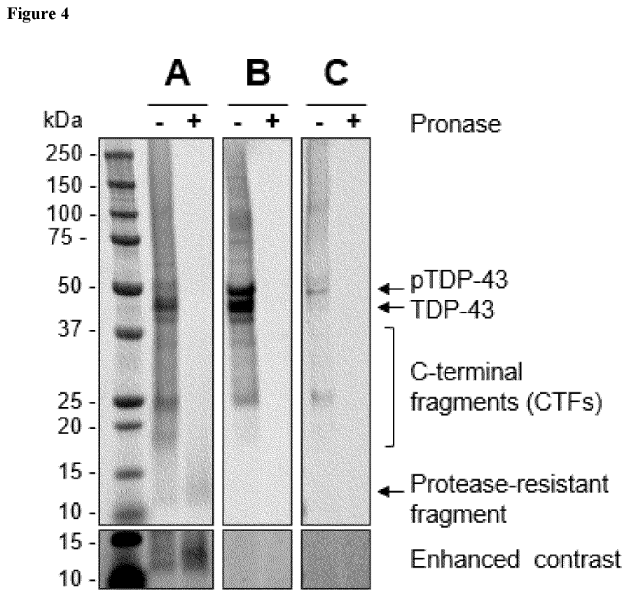

Immunoblot for FTLD-TDP type A sarkosyl-insoluble brain extracts revealed binding of (A) ACI-7071-810H12-Ab1 to C-terminal fragments in addition to the band at 43 kDa corresponding to full-length TDP-43 (FIG. 4). Similar signal was obtained with (C) an antibody binding TDP-43 phosphorylated epitope pS409/410 demonstrating that the C-terminal fragments bound by ACI-7071-810H12-Ab1 retain the disease-specific phosphorylation sites (FIG. 4). However, only ACI-7071-810H12-Ab1 was shown to bind to the protected core of TDP-43 after limited proteolysis of sarkosyl-insoluble FTLD-TDP type A brain extracts using pronase treatment (FIG. 4). In contrast, the antibodies binding either to the N-terminal ((B) TDP-43 antibody binding in RRM2 region) or the C-terminal region (pS409/410 antibody, (C)) of the amyloid core did not show a signal on immunoblots of samples following limited proteolysis.

These data confirm that ACI-7071-810H12-Ab1 binds to the protease-resistant amyloid core of full length and fragments of TDP-43 (expected to be around 8-9 kDa based on the reported structure; Arseni et al., 2021; Arseni et al., 2023). Such binding properties are valuable for use in therapy, as the exposure of the amyloid-core following disease-specific proteolytic cleavage has been shown to further enhance TDP-43 seeding activity (Kumar et al., 2023). In addition, these binding properties are valuable for use in diagnosis, as proteolytic processing of TDP-43 and their enrichment in patient brains has been shown to be a disease-specific pathological signature. In conclusion, these data further support the potential of ACI-7071-810H12-Ab1 for use as a therapeutic or diagnostic antibody.

TABLE 13
Nucleotide sequence of the Heavy Chain (VH) and
Light Chain (VL) Variable Regions

| Antibody | Hybridoma | | |
|---|---|---|---|
| Name | Code | VH | VL |
| ACI- | 704H9 | GAAGTGCAACTGGTGGAGTC | CAACTTGTGCTCACTCAGTC |
| 7071- | A12 | TGGGGGAGACTTAGTGAAGC | ATCTTCAGCCTCTTTCTCCCT |
| 704H9 | | CTGGAGGGTCCCTGAAACTCT | GGGAGCCTCAGCAAAACTC |
| -Ab1 | | CCTGTGCAGCCTCTGGATTCA | ACGTGCACCTTGAGTAGTCA |
| | | CTTTCGCTAACTATGCCATGT | GCACAGTACGTACACCATTG |
| | | CTTGGATTCGCCAGACTCCGG | AATGGTATCAGCAACAGCC |
| | | AAAAGAGGCTGGAGTGGGTC | ACTCAAGCCTCCTAAGTATG |
| | | GCAACCATTTCTGGTGGTGGT | TGATGGAACTTGAGAAAGA |
| | | CTTTATATCAATTATCCAGCC | TGGAAGCCACAGCACAGGT |
| | | AATTTAAAGGGCCGATTCACC | GATGGGATTCCTGATCGCTT |
| | | ATCTCCAGAGACAATGCCAA | TTCTGGATCCAGCTCTGGTG |
| | | GAACAACTTGTACCTGCAAAT | CTGATCGCTACCTTAGCATT |
| | | GAGCCATCTGAAGTCTGAAG | TCCAACATCCAGCCTGAAGA |
| | | ACACAGCCATGTATTACTGTG | TGAAGCAATATACATCTGTG |
| | | TTAAAGATAGGGGCCAAAAC | GTGTGGGTGATACAATTAAG |
| | | TGGTCCTGGTTTGCCTACTGG | GAACAATTTGTGTATGTTTT |
| | | GGCCAAGGGACTCTGGTCAC | CGGCGGTGGAACCAAGGTC |
| | | TGTCTCTGCA | ACTGTCCTA |
| | | (SEQ ID NO: 18) | (SEQ ID NO: 19) |
| ACI- | 707A6 | GAAGTGCAGCTGGTGGAGTC | CAACTTGTGCTCACTCAGTC |
| 7071- | F11 | TGGTGGAGTCTTAGTGAAGCC | ATCTTCAGCCTCTTTCTCCCT |
| 707A6 | | TGGAGGGTCCCTGAAACTCTC | GGGAGCCTCAGCAAAACTC |
| -Ab1 | | CTGTGCAGCCTCTGGATTCAT | ACGTGCACCTTGAGTAGTCA |
| | | TTTCAGTAGCTTTTCCATGTC | GCACAGTACGTACACCATTG |
| | | TTGGGTTCGCCAGAATCCGGA | AATGGTATCAACAACAGCC |
| | | AAAGAGGCTGGAGTGGGTCG | ACTCAAGCCTCCTAAGTATG |
| | | CAACCATTAGTGCTGCTGGTC | TGATGGAGCTTAAGAAAGA |
| | | GTTACATTTACTATCCAGACA | TGGAAGCCACAGCACAGGT |
| | | ATGTTAAGGGCCGATTCACCA | GATGGGATTCCTGATCGCTT |
| | | TCTCCAGAGACAATGCCAAG | CTCTGGATCCAGCTCTGGTG |
| | | AACAACCTGTACCTGTACATG | CTGATCGCTACCTTAGCATT |
| | | AGCCATGTGAGGTCTGAGGA | TCCGACATCCAGCCTGAAGA |
| | | CACAGCCATCTATTACTGTGT | TGAAGCAGTATACATCTGTG |
| | | AAAAGATAGGGGCCTAAACT | GTGTGGGTGATACAATTAAG |
| | | GGTCCTGGTTTGCTTACTGGG | GAACAATTTGTGTATGTTTT |
| | | GCCAAGGGACTCTGGTCACT | CGGCGGTGGAACCAAGGTC |
| | | GTCTCTGCA | ACTGTCCTA |
| | | (SEQ ID NO: 28) | (SEQ ID NO: 29) |
| ACI- | 801H1 | GAGGTCCAGCTACAACAGTC | GATGTTGTGATGACCCAGAC |
| 7071- | E2 | TGGACCTGAGCTGGTGAAGC | TCCACTCACTTTGTCGGTTT |
| 801H1 | | CTGGGGCTTCAGTGAAGATAT | CCATTGGACAACCAGCCTCC |
| -Ab1 | | CCTGCAAGACTTCTGGATACA | ATCTCTTGCAAGTCAAGTCA |
| | | CATTCACTGAATACACCATGC | GAGCCTCTTACATGATGATG |
| | | ACTGGGTGAAGCAGAGCCAT | GAAAGACATATTTGAATTGG |
| | | GGACAGAGCCTTGAATGGAT | TTGTTACAGAGGCCAGGCCA |
| | | TGGAGGTATTCATCCTAACAA | GTCTCCAAAGCGCCTGATCT |
| | | TGGTGGTACTATCTACAACCA | CTCTGGTGTCTAAACTGGAC |
| | | GAAGTTCAAGGGCAGGGCCA | TCTAAATTCCCTGACAGGTT |
| | | CATTGACTGTAGACAAGTCCT | CACTGGCAGTGGATCAGGG |
| | | CCAGCACAGCCTACATGGAG | ACAGATTTCACACTGAAAAT |
| | | TTCCGCAGCCTGACATCTGAT | CAGCAGAGTGGAGGCTGAG |
| | | GATTCTGCAGTCTATTACTGT | GATTTGGGAGTTTATTATTG |
| | | GCAAGTCCCTGCTGGGGTCA | CTGGCAAGGTACACATTTTC |
| | | AGGAACCTCAGTCACCGTCTC | CTCACACGTTCGGTGCTGGG |
| | | CTCA | ACCAAGCTGGAGCTGAAA |
| | | (SEQ ID NO: 38) | (SEQ ID NO: 39) |
| ACI- | 806H5 | GATGTGAAGCTTGAGGACTCT | GATGTTGTGATGACCCAGAT |
| 7071- | B7 | GGAGGAGGCTTGGTGCAACC | TCCACTCACTTTGTCGGTTA |
| 806H5 | | TGAAGGATCCATGAAACTCTC | CCATTGGACAACCAGCCTCC |
| -Ab1 | | TTGTGTTGCCTCTGGATTCAT | ATCTCTTGCAAGTCAAGTCA |
| | | TTTTAGTGACGCCTGGATGGC | GAGCCTCTTAGATAGTGATG |
| | | CTGGGTCCGCCAGTCTCCAGA | GAAAGACATATTTGATTTGG |
| | | AAAGGGGCTTGAGTGGGTTG | TTATTACAGAGGCCAGGCCA |
| | | CTGAAATTAGAGGCAGAGCT | GTCTCCAAAGCGCCTAATCT |
| | | AGTAGTCATGCAACATACTAT | ATCTGGTGTCTAAAAAGGAC |
| | | GCTGAGTCTGTGAAAGGGAG | TCTGGAGTCCCTGACAGGTT |
| | | GTTCACCATCTCAAGAGATGA | CACTGGCAGTGGATCAGGG |
| | | TTCCAAAAGTAGTGTCTACCT | ACAGATTTCACACTGAAAAT |
| | | GCAAATGAACAGCTTAAGAG | CAGCAGAGTGGAGGCTGAG |
| | | CTGAAGACACTGGCGTTTATT | GATCTGGGAGTTTATTATTG |
| | | ACTGTACCAGGGGAGTAAAC | CTGGCAAGGAACACATTTTC |
| | | TACTGGGGCCAAGGCACCGC | CGTGGACGTTCGGTGGAGG |
| | | TCTCACAGTCTCCTCA | CACCAAGCTGGAAATCAAA |
| | | (SEQ ID NO: 48) | (SEQ ID NO: 49) |
| ACI- | 810H1 | CAGGTTCAACTGCAGCAGTCT | GATGTTGTGATGACCCAAAT |
| 7071- | 2B4 | GGGGCTGAGCTGGTGAGGCC | TTCATTCTCCCTGCCTGTCA |
| 810H1 | | TGGGGCTTCAGTGAAGCTGTC | GTCTTGGAGATCAAGCCTCC |
| 2-Ab1 | | CTGCAAGGCTTTGGACTACAC | ATCTCTTGCAGATCTAGTCA |
| | | ATTTACTGACTATGAGATGCA | GAGCCTTGTACACAGTGATG |
| | | CTGGGTGAAGCAGACACCTG | GAACCACCTATTTACATTGG |
| | | TACATGGCCTGGAATGGATTG | TACCTGCAGAAGCCAGGCC |
| | | GAGCGGTTCATCCACGAAGT | AGTCTCCAAAGCTCCTGATC |
| | | GGTGGTACTGTCTATAATCAG | TACAAAGTTTCCAACCGATT |
| | | AGATTCAGGGCCAAGGCCAC | TTCTGGGGTCCCAGACAGGT |
| | | ACTGACTGCTGACAAATCCTC | TCAGTGGCAGTGGATCAGG |
| | | CAGCACAGCCTTCATAGAGCT | GACAGATTTCACACTCAAGA |
| | | CAGCAGCCTGACATCTGAAG | TCAGCAGAGTGGAGGCTGA |
| | | ACTCTGCAGTCTATTTCTGTA | GGATCTGGGAGTTTATTTCT |
| | | CCTACTATGGTTACGACGAAA | GTTCTCAAAGTACACATGCT |
| | | TTTATTGGGGCCAAGGGACTC | CCGTACACGTTCGGAGGGG |
| | | TGGTCACTGTCTCTGCA | GGACCAAGCTGGAAATAAA |
| | | (SEQ ID NO: 58) | A |
| | | | (SEQ ID NO: 59) |

TABLE 14
Amino acid sequence of the Heavy Chain (VH) and Light Chain
(VL) Variable Regions and their CDRs

| Antibody | Hybridoma | | VH | VH | VH | | VL | VL | VL |
|---|---|---|---|---|---|---|---|---|---|
| name | code | VH | CDR1 | CDR2 | CDR3 | VL | CDR1 | CDR2 | CDR3 |
| ACI- | 704H9 | EVQLVES | NY | TIS | DRG | QLVLTQSS | TL | LEK | GVG |
| 7071- | A12 | GGDLVKP | AM | GG | QN | SASFSLGA | SS | DGS | DTI |
| 704H | | GGSLKLSC | S | GLY | WS | SAKLTCTL | QH | HST | KEQ |
| 9- | | AASGFTFA | (SEQ | INY | WF | SSQHSTYT | ST | GD | FVY |
| Ab1 | | NYAMSWI | ID | PAN | AY | IEWYQQQ | YTI | (SEQ | V |
| | | RQTPEKRL | NO: | LKG | (SEQ | PLKPPKYV | E | ID | (SEQ |
| | | EWVATIS | 11) | (SEQ | ID | MELEKDG | (SEQ | NO: | ID |
| | | GGGLYIN | | ID | NO: | SHSTGDGI | ID | 16) | NO: |
| | | YPANLKG | | NO: | 13) | PDRFSGSS | NO: | | 17) |
| | | RFTISRDN | | 12) | | SGADRYL | 15) | | |
| | | AKNNLYL | | | | SISNIQPED | | | |
| | | QMSHLKS | | | | EAIYICGV | | | |
| | | EDTAMYY | | | | GDTIKEQF | | | |
| | | CVKDRGQ | | | | VYVFGGG | | | |
| | | NWSWFAY | | | | TKVTVL | | | |
| | | WGQGTLV | | | | (SEQ ID | | | |
| | | TVSA | | | | NO: 14) | | | |
| | | (SEQ ID | | | | | | | |
| | | NO: 10) | | | | | | | |
| ACI- | 707A6 | EVQLVES | SFS | TIS | DRG | QLVLTQSS | TL | LKK | GVG |
| 7071- | F11 | GGVLVKP | MS | AA | LN | SASFSLGA | SS | DGS | DTI |
| 707A | | GGSLKLSC | (SEQ | GRY | WS | SAKLTCTL | QH | HST | KEQ |
| 6- | | AASGFIFS | ID | IYY | WF | SSQHSTYT | ST | GD | FVY |
| Ab1 | | SFSMSWV | NO: | PDN | AY | IEWYQQQ | YTI | (SEQ | V |
| | | RQNPEKR | 21) | VK | (SEQ | PLKPPKYV | (SEQ | ID | (SEQ |
| | | LEWVATIS | | G | ID | MELKKDG | ID | NO: | ID |
| | | AAGRYIY | | (SEQ | NO: | SHSTGDGI | NO: | 26) | NO: |
| | | YPDNVKG | | ID | 23) | PDRFSGSS | 15) | | 17) |
| | | RFTISRDN | | NO: | | SGADRYL | | | |
| | | AKNNLYL | | 22) | | SISDIQPED | | | |
| | | YMSHVRS | | | | EAVYICG | | | |
| | | EDTAIYYC | | | | VGDTIKEQ | | | |
| | | VKDRGLN | | | | FVYVFGG | | | |
| | | WSWFAY | | | | GTKVTVL | | | |
| | | WGQGTLV | | | | (SEQ ID | | | |
| | | TVSA | | | | NO: 24) | | | |
| | | (SEQ ID | | | | | | | |
| | | NO: 20) | | | | | | | |
| ACI- | 801H1 | EVQLQQS | EYT | GIH | PC | DVVMTQT | KS | LVS | WQ |
| 7071- | E2 | GPELVKP | MH | PNN | | PLTLSVSI | SQ | KLD | GTH |
| 801H | | GASVKISC | (SEQ | GGT | | GQPASISC | SL | S | FPH |
| 1- | | KTSGYTFT | ID | IYN | | KSSQSLLH | LH | (SEQ | T |
| Ab1 | | EYTMHW | NO: | QKF | | DDGKTYL | DD | ID | (SEQ |
| | | VKQSHGQ | 31) | KG | | NWLLQRP | GK | NO: | ID |
| | | SLEWIGGI | | (SEQ | | GQSPKRLI | TY | 36) | NO: |
| | | HPNNGGTI | | ID | | SLVSKLDS | LN | | 37) |
| | | YNQKFKG | | NO: | | KFPDRFTG | (SEQ | | |
| | | RATLTVD | | 32) | | SGSGTDFT | ID | | |
| | | KSSSTAY | | | | LKISRVEA | NO: | | |
| | | MEFRSLTS | | | | EDLGVYY | 35) | | |
| | | DDSAVYY | | | | CWQGTHF | | | |
| | | CASPCWG | | | | PHTFGAG | | | |
| | | QGTSVTV | | | | TKLELK | | | |
| | | SS | | | | (SEQ ID | | | |
| | | (SEQ ID | | | | NO: 34) | | | |
| | | NO: 30) | | | | | | | |
| ACI- | 806H5 | DVKLEDS | DA | EIR | GV | DVVMTQI | KS | LVS | WQ |
| 7071- | B7 | GGGLVQP | WM | GRA | NY | PLTLSVTI | SQ | KK | GTH |
| 806H | | EGSMKLS | A | SSH | (SEQ | GQPASISC | SL | DS | FPW |
| 5- | | CVASGFIF | (SEQ | ATY | ID | KSSQSLLD | LD | (SEQ | T |
| Ab1 | | SDAWMA | ID | YAE | NO: | SDGKTYLI | SD | ID | (SEQ |
| | | WVRQSPE | NO: | SVK | 43) | WLLQRPG | GK | NO: | ID |
| | | KGLEWVA | 41) | G | | QSPKRLIY | TY | 46) | NO: |
| | | EIRGRASS | | (SEQ | | LVSKKDS | LI | | 47) |
| | | HATYYAE | | ID | | GVPDRFT | (SE | | |
| | | SVKGRFTI | | NO: | | GSGSGTDF | Q | | |
| | | SRDDSKSS | | 42) | | TLKISRVE | ID | | |
| | | VYLQMNS | | | | AEDLGVY | NO: | | |
| | | LRAEDTG | | | | YCWQGTH | 45) | | |
| | | VYYCTRG | | | | FPWTFGG | | | |
| | | VNYWGQ | | | | GTKLEIK | | | |
| | | GTALTVSS | | | | (SEQ ID | | | |
| | | (SEQ ID | | | | NO: 44) | | | |
| | | NO: 40) | | | | | | | |
| ACI- | 810H1 | QVQLQQS | DYE | AV | YG | DVVMTQI | RS | KVS | SQS |
| 7071- | 2B4 | GAELVRP | MH | HPR | YDE | SFSLPVSL | SQ | NRF | THA |
| 810H | | GASVKLS | (SEQ | SGG | IY | GDQASISC | SL | S | PYT |
| 12- | | CKALDYT | ID | TVY | (SEQ | RSSQSLVH | VH | (SEQ | (SEQ |
| Ab1 | | FTDYEMH | NO: | NOR | ID | SDGTTYL | SD | ID | ID |
| | | WVKQTPV | 51) | FRA | NO: | HWYLQKP | GT | NO: | NO: |
| | | HGLEWIG | | (SEQ | 53) | GQSPKLLI | TY | 56) | 57) |
| | | AVHPRSG | | ID | | YKVSNRF | LH | | |
| | | GTVYNQR | | NO: | | SGVPDRFS | (SEQ | | |
| | | FRAKATL | | 52) | | GSGSGTDF | ID | | |
| | | TADKSSST | | | | TLKISRVE | NO: | | |
| | | AFIELSSLT | | | | AEDLGVY | 55) | | |
| | | SEDSAVYF | | | | FCSQSTHA | | | |
| | | CTYYGYD | | | | PYTFGGG | | | |
| | | EIYWGQG | | | | TKLEIK | | | |
| | | TLVTVSA | | | | (SEQ ID | | | |
| | | (SEQ ID | | | | NO: 54) | | | |
| | | NO: 50) | | | | | | | |

### REFERENCES

Afroz, Tariq, Manuela Pérez-Berlanga, and Magdalini Polymenidou. “Structural transition, function and dysfunction of TDP-43 in neurodegenerative diseases.” Chimia 73.5 (2019): 380-380.
Arai et al., TDP-43 is a component of ubiquitin-positive tau-negative inclusions in frontotemporal lobar degeneration and amyotrophic lateral sclerosis, Biochemical and Biophysical Research Communications 351 (2006) 602-611.
Arseni D, Chen R, Murzin A G, Peak-Chew S Y, Garringer H J, Newell K L, Kametani F, Robinson A C, Vidal R, Ghetti B et al (2023) TDP-43 forms amyloid filaments with a distinct fold in type A FTLD-TDP. Nature 620: 898-903 Doi 10.1038/s41586-023-06405-w
Arseni D, Hasegawa M, Murzin A G, Kametani F, Arai M, Yoshida M, Ryskeldi-Falcon B (2021) Structure of pathological TDP-43 filaments from ALS with FTLD. Nature: Doi 10.1038/s41586-021-04199-3.
Audrain, Mickael, Anne-Laure Egesipe, Noémie Tentillier, Laure Font, Monisha Ratnam, Lorene Mottier, Mathieu Clavel et al. “Targeting amyotrophic lateral sclerosis by neutralizing seeding-competent TDP-43 in CSF.” Brain Communications 5, no. 6 (2023): fcad306.
Betts et al., Linear pharmacokinetic parameters for monoclonal antibodies are similar within a species and across different pharmacological targets: A comparison between human, cynomolgus monkey and hFcRn Tg32 transgenic mouse using a population-modeling approach, MABS, 2018, Vol. 10
Buratti and Baralle, Nuclear factor TDP-43 can affect selected microRNA levels, FEBS Journal 277(2010)2268-2281.
Brettschneider J et al., Spreading of pathology in neurodegenerative diseases: a focus on human studies, Nature Rev. Neuroscience, 2015, 109.
Brettschneider et al., Stages of pTDP-43 pathology in amyotrophic lateral sclerosis, Ann Neurol. 2013 July; 74(1): 20-38.
Charlton, Methods in Molecular Biology, Val. 248 (B. K. C. Lo, ed., Humana Press, Totowa, NJ, 2003), pp. 245-254.
Chowdhury, Methods Mol. Biol. 207:179-196 (2008)
Clynes et al., Fc receptors are required in passive and active immunity to melanoma Proc. Nat'l Acad. sci. USA 95:652-656 (1998)
Cohen et al., An acetylation switch controls TDP-43 function and aggregation propensity, Nat Commun. 6: 5845, 2015.
Cragg, M. S. et al., Complement-mediated lysis by anti-CD20 mAb correlates with segregation into lipid rafts, Blood 101:1045-1052 (2003)
Cragg, M. S. and M. J. Glennie, Antibody specificity controls in vivo effector mechanisms of anti-CD20 reagents, Blood 103:2738-2743 (2004)).
Cunningham and Wells, High-resolution epitope mapping of hGH-receptor interactions by alanine-scanning mutagenesis, (1989) Science, 244: 1081-1085.
Dall' Acqua et al., Increasing the Affinity of a Human IgG1 for the Neonatal Fc Receptor: Biological Consequences, Journal of immunology, (2002) 169(9):5171-5180.
Duncan & Winter, The binding site for C1q on IgG, Nature 322:738-40 (1988)
Edelman, G. M. et al., The Covalent Structure of an Entire gammaG Immunoglobulin Molecule, Proc. Natl. Acad. USA, 63, 78-85 (1969)
Feiler et al., TDP-43 is intercellularly transmitted across axon Terminals, J. Cell Biol. Vol. 211 No. 4 897-911.
Feneberg et al., Towards a TDP-43-Based Biomarker for ALS and FTLD, Molecular Neurobiology, 2018; 55(10): 7789-7801.
Gasset-Rosa F, Lu S, Yu H, Chen C, Melamed Z, Guo L, Shorter J, Da Cruz S, Cleveland D W (2019) Cytoplasmic TDP-43 De-mixing Independent of Stress Granules Drives Inhibition of Nuclear Import, Loss of Nuclear TDP-43, and Cell Death. Neuron 102: 339-357 e337 Doi 10.1016/j.neuron.2019.02.038
Gazzano-Santoro et al., Engineered Antibodies with Increased Activity to Recruit Complement, J. Immunol. Methods 202:163 (1996)
Gerngross, Advances in the production of human therapeutic proteins in yeasts and filamentous fungi, Nat. Biotech. 22:1409-1414 (2004).
Gerhardt et al., Methods for General and Molecular Bacteriology, ASM Press (1994).
Golemis, Protein-Protein Interactions: A Molecular Cloning Manual, Cold Spring Harbor Laboratory Press (2002).
Graham et al., Characteristics of a human cell line transformed by DNA from human adenovirus type 5, J. Gen Viral. 36:59 (1977)
Harlow and Lane, Antibodies: A Laboratory Manual, Cold Spring Harbor Laboratory Press, (1988).
Harlow and Lane, Using Antibodies: A Laboratory Manual, Cold Spring Harbor Laboratory Press, (1999).
Hasegawa et al., Phosphorylated TDP-43 in frontotemporal lobar degeneration and amyotrophic lateral sclerosis, (2008) Annals of Neurology Vol 64 No 1, 60-70.
Hasegawa et al., Prion-like mechanisms and potential therapeutic targets in neurodegenerative disorders, Pharmacol Ther. 2017 April; 172:22-33.
Hellstrom, I. et al., Proc. Nat'l Acad. Sci. USA 83:7059-7063 (1986).
Hellstrom, I et al., Proc. Nat'l Acad. Sci. USA 82:1499-1502 (1985).
Hoogenboom et al., Methods in Molecular Biology 178:1-37 (O'Brien et al., ed., Human Press, Totowa, NJ, (2001).
Howard and Bethell. (2000) Basic Methods in Antibody Production and Characterization, Crc. Pr. Inc.
Ishii et al., Formation and spreading of TDP-43 aggregates in cultured neuronal and glial cells demonstrated by time-lapse imaging, PLoS ONE 12(6): e0179375, 2017.
Jones et al., Replacing the complementarity-determining regions in a human antibody with those from a mouse, Nature 321 (1986), 522-525.
K A et al., TDP-43 is a key player in the clinical features associated with Alzheimer's disease, Acta Neuropathol. 2014; 127(6): 811-824.
K A et al., Staging TDP-43 pathology in Alzheimer's disease Acta Neuropathol. 2014; 127(3): 441-450.
Kam et al., Proc. Natl. Acad. Sci. USA 102: 11600-11605 (2005).
Kanda Y. et al., Bioteehnol. Bioeng., 94(4):680-688 (2006).
Kabat et al., Sequences of Proteins of Immunological Interest, 5th edit. NIH Publication no. 91-3242 U.S. Department of Health and Human Services (1991).
Kasai et al. “Combined use of CSF NfL and CSF TDP-43 improves diagnostic performance in ALS.” Annals of clinical and translational neurology vol. 6,12 (2019): 2489-2502. doi:10.1002/acn3.50943
Kohler, Nature 256 (1975), 495.
Khor, Proceedings of the American Society of Clinical Oncology Abstract (1997), 847.
Kumar S T, Nazarov S, Porta S, Maharjan N, Cendrowska U, Kabani M, Finamore F, Xu Y, Lee V M, Lashuel H A (2023) Seeding the aggregation of TDP-43 requires post-fibrillization proteolytic cleavage. Nat Neurosci 26: 983-996 Doi 10.1038/s41593-023-01341-4.
Laferriere F, Maniecka Z, Perez-Berlanga M, Hruska-Plochan M, Gilhespy L, Hock E M, Wagner U, Afroz T, Boersema P J, Barmettler G et al (2019) TDP-43 extracted from frontotemporal lobar degeneration subject brains displays distinct aggregate assemblies and neurotoxic effects reflecting disease progression rates. Nat Neurosci 22: 65-77 Doi 10.1038/s41593-018-0294-y
Lagier-Tourenne et al., TDP-43 and FUS/TLS: emerging roles in RNA processing and neurodegeneration, Human Molecular Genetics, 2010, Vol. 19, Review Issue 1 R46-R64.
Lagier-Tourenne and Cleveland, Rethinking ALS: the FUS about TDP-43, Cell 136, 2009, 1001-1004.
Le Ber, Genetics of frontotemporal lobar degeneration: an up-date and diagnosis algorithm, Revue Neurologique 169 (2013) 811-819.
Lefkovits, Immunology Methods Manual: The Comprehensive Sourcebook of Techniques; Academic Press (1997).
LoBuglio, Proceedings of the American Society of Clinical Oncology Abstract (1997), 1562.
Li et al., Optimization of humanized IgGs in glycoengineered Pichia pastoris, Nat. Biotech. 24:210-215 (2006).
Mackenzie and Neumann, Molecular neuropathology of frontotemporal dementia: insights into disease mechanisms from postmortem studies, J. Neurochem. (2016) 138 (Suppl. 1), 54-70.
Mather, Establishment and characterization of two distinct mouse testicular epithelial cell lines, Biol. Reprod. 23:243-251 (1980)
Mather et al., Culture of testicular cells in hormone-supplemented serum-free medium, Annals N. Y Aead. Sei. 383:44-68 (1982)
McAleese et al., TDP-43 pathology in Alzheimer's disease, dementia with Lewy bodies and ageing, Brain Pathol. 2017 July; 27(4): 472-479.
Nonaka et al., Prion-like Properties of Pathological TDP-43 Aggregates from Diseased Brains, Cell Reports 4 (2013), 124-134.
Neumann et al., Ubiquitinated TDP-43 in frontotemporal lobar degeneration and amyotrophic lateral sclerosis, Science 314, (2006), 130-133.
Neumann et al., Phosphorylation of S409/410 of TDP-43 is a consistent feature in all sporadic and familial forms of TDP-43 proteinopathies, Acta Neuropathol. (2009) 117: 137-149.
Petkova, S. B. et al., Int'l. Immunol. 18(12):1759-1769 (2006).
Plückthun, The Pharmacology of Monoclonal Antibodies, Rosenburg and Moore eds. Springer-Verlag, N.Y. (1994), 269-315.
Porta S. et al., Patient-derived frontotemporal lobar degeneration brain extracts induce formation and spreading of TDP-43 pathology in vivo Nat. Comm., 2018
Okazaki et al., Fucose depletion from human IgG1 oligosaccharide enhances binding enthalpy and association rate between IgG1 and FcgammaRIIIa. J. Mol. Biol. 336:1239-1249 (2004).
Presta L G., Antibody engineering, Curr Op Struct Biol 2 (1992), 593-596.
Ravetch and Kinet, Annu. Rev. Immunol. 9:457-492 (1991)
Reichmann, Reshaping human antibodies for therapy, Nature 332 (1998), 323-327.
Remington's Pharmaceutical Sciences 16th edition, Osol, A. Ed. (1980)
Ripka et al., Two Chinese hamster ovary glycosylation mutants affected in the conversion of GDP-mannose to GDP-fucose. Arch. Biochem. Biophys. 249:533-545 (1986).
Sambrook et al., Molecular Cloning: A Laboratory Manual; Cold Spring Harbor Laboratory Press, 2nd edition (1989) and 3rd edition (2001).
Shepherd and Dean (2000), Monoclonal Antibodies: A Practical Approach, Oxford University Press
Shields et al., High Resolution Mapping of the Binding Site on Human IgG1 for FcγRI, FcγRII, FcγRIII, and FcRn and Design of IgG1 Variants with Improved Binding to the FcγR, J. Biol. Chem. 9(2): 6591-6604 (2001).
Ticozzi et al., Protein Aggregation and Defective RNA Metabolism as Mechanisms for Motor Neuron Damage, 9(3): 285-296 CNS Neurol. Disord. Drug Targets. 2010, 9(3), 285-296.
Urlaub et al., Isolation of Chinese hamster cell mutants deficient in dihydrofolate reductase activity, Proc. Natl. Acad. cii. USA 77:4216 (1980)
Verhoeyen et al., Reshaping human antibodies: Grafting an antilysozyme activity., Science 239 (1988), 1534-1536.
Wang A, Conicella A E, Schmidt H B, Martin E W, Rhoads S N, Reeb A N, et al. A single N-terminal phosphomimic disrupts TDP-43 polymerization, phase separation, and RNA splicing. EMBO J. 2018; 37(5).
Wang et al., TDP-43: an emerging new player in neurodegenerative diseases Trends in Molecular Medicine Vol. 14 No. 11, 2008, 479-485.
Warraich et al., TDP-43: a DNA and RNA binding protein with roles in neurodegenerative diseases, The International Journal of Biochemistry & Cell Biology 42 (2010) 1606-1609.Wright et al., Effect of glycosylation on antibody function. TIBTECH 15:26-32 (1997).
Yamane-Ohnuki et al., Establishment of FUT8 knockout Chinese hamster ovary cells: An ideal host cell line for producing completely defucosylated antibodies with enhanced antibody-dependent cellular cytotoxicity. Biotech. Bioeng. 87: 614 (2004).
Yazaki and Wu, Methods in Molecular Biology, Val. 248 (B. K. C. Lo, ed., Humana Press, Totowa, NJ), pp. 255-268 (2003).
Zalevsky, J et al, Enhanced antibody half-life improves in vivo activity, Nat Biotechnol 28(2):157-9, 2010.

Unless defined otherwise, all technical and scientific terms used herein have the same meanings as commonly understood by one of ordinary skill in the art to which this invention belongs. All publications and patents specifically mentioned herein are incorporated by reference in their entirety for all purposes in connection with the invention.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. Moreover, all aspects and embodiments of the invention described herein are considered to be broadly applicable and combinable with any and all other consistent embodiments, including those taken from other aspects of the invention (including in isolation) as appropriate.

1. A TDP-43 binding molecule, in particular a TDP-43 antibody or an antigen-binding fragment thereof, comprising:
a. a Heavy Chain Variable Region (VH) which comprises a VH-CDR1 comprising the amino acid sequence of SEQ ID NO: 51, a VH-CDR2 comprising the amino acid sequence of SEQ ID NO: 52 and a VH-CDR3 comprising the amino acid sequence of SEQ ID NO: 53; and a Light Chain Variable Region (VL) which comprises a VL-CDR1 comprising the amino acid sequence of SEQ ID NO: 55, a VL-CDR2 comprising the amino acid sequence of SEQ ID NO: 56 and a VL-CDR3 comprising the amino acid sequence of SEQ ID NO: 57; or
b. a Heavy Chain Variable Region (VH) which comprises a VH-CDR1 comprising the amino acid sequence of SEQ ID NO: 41, a VH-CDR2 comprising the amino acid sequence of SEQ ID NO: 42 and a VH-CDR3 comprising the amino acid sequence of SEQ ID NO: 43; and a Light Chain Variable Region (VL) which comprises a VL-CDR1 comprising the amino acid sequence of SEQ ID NO: 45, a VL-CDR2 comprising the amino acid sequence of SEQ ID NO: 46 and a VL-CDR3 comprising the amino acid sequence of SEQ ID NO: 47; or
c. a Heavy Chain Variable Region (VH) which comprises a VH-CDR1 comprising the amino acid sequence of SEQ ID NO: 31, a VH-CDR2 comprising the amino acid sequence of SEQ ID NO: 32 and a VH-CDR3 comprising the amino acid sequence PC (Pro-Cys); and a Light Chain Variable Region (VL) which comprises a VL-CDR1 comprising the amino acid sequence of SEQ ID NO: 35, a VL-CDR2 comprising the amino acid sequence of SEQ ID NO: 36 and a VL-CDR3 comprising the amino acid sequence of SEQ ID NO: 37; or
d. a Heavy Chain Variable Region (VH) which comprises a VH-CDR1 comprising the amino acid sequence of SEQ ID NO: 21, a VH-CDR2 comprising the amino acid sequence of SEQ ID NO: 22 and a VH-CDR3 comprising the amino acid sequence of SEQ ID NO: 23; and a Light Chain Variable Region (VL) which comprises a VL-CDR1 comprising the amino acid sequence of SEQ ID NO: 15, a VL-CDR2 comprising the amino acid sequence of SEQ ID NO: 26 and a VL-CDR3 comprising the amino acid sequence of SEQ ID NO: 17; or
e. a Heavy Chain Variable Region (VH) which comprises a VH-CDR1 comprising the amino acid sequence of SEQ ID NO: 11, a VH-CDR2 comprising the amino acid sequence of SEQ ID NO: 12 and a VH-CDR3 comprising the amino acid sequence of SEQ ID NO: 13; and a Light Chain Variable Region (VL) which comprises a VL-CDR1 comprising the amino acid sequence of SEQ ID NO: 15, a VL-CDR2 comprising the amino acid sequence of SEQ ID NO: 16 and a VL-CDR3 comprising the amino acid sequence of SEQ ID NO: 17.

2. The TDP-43 binding molecule of claim 1, comprising:
a. a Heavy Chain Variable Region (VH) comprising the sequence of SEQ ID NO: 50 or a Heavy Chain Variable Region (VH) having at least 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 50; and a Light Chain Variable Region (VL) comprising the sequence of SEQ ID NO: 54 or a Light Chain Variable Region (VL) having at least 96%, 97%, 98% or 99% sequence identity to the amino acid sequence of SEQ ID NO: 54; or
b. a Heavy Chain Variable Region (VH) comprising the sequence of SEQ ID NO: 40 or a Heavy Chain Variable Region (VH) having at least 86%, 87%, 88%, 89%, 90%, 91%, 92%, 93%, 94%, 95%, 96%, 97%, 98% or 99% sequence identity to the amino acid sequence of SEQ ID NO: 40; and a Light Chain Variable Region (VL) comprising the sequence of SEQ ID NO: 44 or a Light Chain Variable Region (VL) having at least 98% or 99% sequence identity to the amino acid sequence of SEQ ID NO: 44; or
c. a Heavy Chain Variable Region (VH) comprising the sequence of SEQ ID NO: 30 or a Heavy Chain Variable Region (VH) having at least 90%, 91%, 92%, 93%, 94%, 95%, 96%, 97%, 98% or 99% sequence identity to the amino acid sequence of SEQ ID NO: 30; and a Light Chain Variable Region (VL) comprising the sequence of SEQ ID NO: 34 or a Light Chain Variable Region (VL) having at least 94%, 95%, 96%, 97%, 98% or 99% sequence identity to the amino acid sequence of SEQ ID NO: 34; or
d. a Heavy Chain Variable Region (VH) comprising the sequence of SEQ ID NO: 20 or a Heavy Chain Variable Region (VH) having at least 84%, 85%, 86%, 87%, 88%, 89%, 90%, 91%, 92%, 93%, 94%, 95%, 96%, 97%, 98% or 99% sequence identity to the amino acid sequence of SEQ ID NO: 20; and a Light Chain Variable Region (VL) comprising the sequence of SEQ ID NO: 24 or a Light Chain Variable Region (VL) having at least 99% sequence identity to the amino acid sequence of SEQ ID NO: 24; or
e. a Heavy Chain Variable Region (VH) comprising the sequence of SEQ ID NO: 10 or a Heavy Chain Variable Region (VH) having at least 87%, 88%, 89%, 90%, 91%, 92%, 93%, 94%, 95%, 96%, 97%, 98% or 99% sequence identity to the amino acid sequence of SEQ ID NO: 10; and a Light Chain Variable Region (VL) comprising the sequence of SEQ ID NO: 14.

3. The TDP-43 binding molecule of any one of the preceding claims, comprising:
a. a Heavy Chain Variable Region (VH) comprising the amino acid sequence of SEQ ID NO: 50 and a Light Chain Variable Region (VL) comprising the amino acid sequence of SEQ ID NO: 54; or
b. a Heavy Chain Variable Region (VH) comprising the amino acid sequence of SEQ ID NO: 40 and a Light Chain Variable Region (VL) comprising the amino acid sequence of SEQ ID NO: 44; or
c. a Heavy Chain Variable Region (VH) comprising the amino acid sequence of SEQ ID NO: 30 and a Light Chain Variable Region (VL) comprising the amino acid sequence of SEQ ID NO: 34; or
d. a Heavy Chain Variable Region (VH) comprising the amino acid sequence of SEQ ID NO: 20 and a Light Chain Variable Region (VL) comprising the amino acid sequence of SEQ ID NO: 24; or
e. a Heavy Chain Variable Region (VH) comprising the amino acid sequence of SEQ ID NO: 10 and a Light Chain Variable Region (VL) comprising the amino acid sequence of SEQ ID NO: 14.

4. The TDP-43 binding molecule of any one of the preceding claims, which binds misfolded aggregated TDP-43 and non-aggregated physiological TDP-43.

5. The TDP-43 binding molecule of any one of the preceding claims, which binds to monomeric and/or oligomeric and/or aggregated and/or post-translationally modified and/or truncated TDP-43, preferably wherein the TDP-43 is human TDP-43.

6. The TDP-43 binding molecule of any one of the preceding claims, which binds misfolded aggregated human TDP-43 and non-aggregated physiological human TDP-43.

7. TDP-43 binding molecule of any one of the preceding claims, which exhibits one or more, up to all of the following characteristics:
a. inhibits the aggregation of TDP-43 protein or fragments thereof;
b. blocks TDP-43 cell-to-cell propagation;
c. disaggregates TDP-43 aggregates;
d. blocks TDP-43 seeding;
e. neutralizes seeding-competent TDP-43;
f. blocks TDP-43 spreading;
g. potentiates TDP-43 clearance; and
h. reduces phosphorylated TDP-43 level in vivo.

8. The TDP-43 binding molecule of any one of the preceding claims, which exhibits one or more, up to all of the following characteristics:
a. inhibits the aggregation of TDP-43 protein or fragments thereof;
b. blocks TDP-43 cell-to-cell propagation;
c. blocks TDP-43 seeding;
d. blocks TDP-43 spreading;
e. potentiates TDP-43 clearance; and
f. reduces phosphorylated TDP-43 level in vivo.

9. The TDP-43 binding molecule of any one of the preceding claims, which potentiates TDP-43 clearance.

10. The TDP-43 binding molecule of any one of the preceding claims, which reduces TDP-43 pathology in vivo.

11. The TDP-43 binding molecule of any one of the preceding claims, which reduces levels of misfolded aggregated TDP-43 and/or phosphorylated TDP-43 in vivo.

12. The TDP-43 binding molecule of any one of the preceding claims, which reduces levels of phosphorylated TDP-43 in the hippocampus.

13. The TDP-43 binding molecule of any one of the preceding claims, which binds to an epitope within amino acid residues 304-414 of human TDP-43 (SEQ ID NO: 1).

14. The TDP-43 binding molecule of any one of the preceding claims, which binds to an epitope within amino acid residues 304-313 of human TDP-43 (SEQ ID NO: 1).

15. The TDP-43 binding molecule of any one of claims 1 to 13, which binds to an epitope within amino acid residues 396-414 of human TDP-43 (SEQ ID NO: 1).

16. The TDP-43 binding molecule of any one of claims 1 to 14, which binds to the protease-resistant amyloid core of TDP-43.

17. The TDP-43 binding molecule of any one of the preceding claims, which is an antibody or an antigen-binding fragment thereof.

18. The TDP-43 binding molecule of any one of the preceding claims, which has a dissociation constant (KD) for binding soluble TDP-43 (SEQ ID NO: 1) of 1 nM or less, preferably 750 pM or less, 500 pM or less, 380 pM or less, 230 pM or less, 200 pM or less, or 110 pM or less.

19. The TDP-43 binding molecule of any one of the preceding claims, which is an IgA, IgD, IgE, IgM, IgG1, IgG2, IgG3 or IgG4 antibody or antigen-binding fragment thereof.

20. The TDP-43 binding molecule of any one of the preceding claims, which is an IgG1 or an IgG4 antibody or antigen-binding fragment thereof.

21. The TDP-43 binding molecule of any one of the preceding claims which comprises an Fc mutation, preferably the S228P mutation.

22. An immunoconjugate comprising the TDP-43 binding molecule according to any one of the preceding claims.

23. The immunoconjugate of claim 22, wherein the immunoconjugate crosses the blood brain barrier using a delivery vehicle or a blood brain barrier moiety.

24. The immunoconjugate of claim 23, wherein the delivery vehicle comprises a liposome or extracellular vesicle.

25. The immunoconjugate of claim 23, wherein the TDP-43 binding molecule is linked to the blood brain barrier moiety.

26. The immunoconjugate of claim 23 or 25, wherein the blood brain barrier moiety is a polypeptide or a small molecule, preferably, a peptide, a receptor ligand, a single domain antibody (VHH), a scFv or a Fab fragment.

27. The immunoconjugate of claim 23, 25 or 26, wherein the blood brain barrier moiety binds a blood brain barrier receptor.

28. The immunoconjugate of claim 27, wherein the blood brain barrier receptor comprises a transferrin receptor, insulin receptor or low-density lipoprotein receptor.

29. A labelled binding molecule, in particular a labelled antibody, comprising the TDP-43 binding molecule according to any one of claims 1 to 21.

30. A pharmaceutical composition comprising the TDP-43 binding molecule of any one of claims 1 to 21, or an immunoconjugate as claimed in any one of claims 22 to 28 and a pharmaceutically acceptable carrier and/or excipient and/or diluent.

31. The TDP-43 binding molecule of any one of claims 1 to 21, or an immunoconjugate as claimed in any one of claims 22 to 28 or pharmaceutical composition of claim 30 for human or veterinary medicine use.

32. The TDP-43 binding molecule of any one of claims 1 to 21, or an immunoconjugate as claimed in any one of claims 22 to 28 or pharmaceutical composition of claim 30 for use in the prevention, alleviation, treatment of diseases, disorders and/or abnormalities associated with TDP-43 or TDP-43 proteinopathy.

33. The TDP-43 binding molecule of any one of claims 1 to 21 or an immunoconjugate as claimed in any one of claims 22 to 28, labelled binding molecule of claim 29 or pharmaceutical composition of claim 30 for diagnostic use.

34. The TDP-43 binding molecule or immunoconjugate, labelled binding molecule or pharmaceutical composition for use as claimed in claim 33 for diagnosis of diseases, disorders and/or abnormalities associated with TDP-43 or TDP-43 proteinopathy.

35. The TDP-43 binding molecule of any one of claims 1 to 21 or an immunoconjugate as claimed in any one of claims 22 to 28, labelled binding molecule of claim 29 or pharmaceutical composition of claim 30 for research use, in particular as an analytical tool or reference molecule.

36. The TDP-43 binding molecule of any one of claims 1 to 21 or an immunoconjugate as claimed in any one of claims 22 to 28, labelled binding molecule of claim 29 or pharmaceutical composition of claim 30 for use as a diagnostic tool to monitor diseases, disorders and/or abnormalities associated with TDP-43 or TDP-43 proteinopathy.

37. The TDP-43 binding molecule or immunoconjugate or labelled binding molecule or pharmaceutical composition for use according to any one of claims 32, 34 or 36, wherein the disease, disorder and/or abnormality associated with TDP-43, or TDP-43 proteinopathy, is Frontotemporal dementia (FTD, such as sporadic or familial with or without motor-neuron disease (MND), with progranulin (GRN) mutation, with C9orf72 mutations, with TARDBP mutation, with valosin-containing protein (VCP) mutation, linked to chromosome 9p, corticobasal degeneration, frontotemporal lobar degeneration (FTLD) with ubiquitin-positive TDP-43 inclusions (FTLD-TDP), Argyrophilic grain disease, Pick's disease, semantic variant Primary Progressive Aphasia (svPPA), behavioural variant FTD (bvFTD), nonfluent variant Primary Progressive Aphasia (nfvPPA) and the like), Amyotrophic lateral sclerosis (ALS, such as sporadic ALS, with TARDBP mutation, with angiogenin (ANG) mutation), Alexander disease (AxD), limbic-predominant age-related TDP-43 encephalopathy (LATE), Chronic Traumatic Encephalopathy (CTE), Perry syndrome, Alzheimer's disease (AD, including sporadic and familial forms of AD), Down syndrome, Familial British dementia, Polyglutamine diseases (Huntington's disease and spinocerebellar ataxia type 3 (SCA3; also known under Machado Joseph Disease)), Hippocampal sclerosis dementia and Myopathies (sporadic inclusion body myositis, Inclusion body myopathy with a mutation in the valosin-containing protein ((VCP); associated with Paget disease of bone and frontotemporal dementia), Oculo-pharyngeal muscular dystrophy with rimmed vacuoles, Myofibrillar myopathies with mutations in the myotilin (MYOT) gene or mutations in the gene coding for desmin (DES)), Traumatic Brain Injury (TBI), Dementia with Lewy Bodies (DLB) or Parkinson's disease (PD).

38. The TDP-43 binding molecule or immunoconjugate or labelled binding molecule or pharmaceutical composition for use according to claim 37, wherein the disease, disorder and/or abnormality associated with TDP-43, or TDP-43 proteinopathy, is Frontotemporal dementia (FTD), amyotrophic lateral sclerosis (ALS), Alzheimer's disease (AD), Parkinson's disease (PD), Chronic Traumatic Encephalopathy (CTE), or limbic-predominant age-related TDP-43 encephalopathy (LATE).

39. The TDP-43 binding molecule or immunoconjugate or labelled binding molecule or pharmaceutical composition for use according to claim 38, wherein the disease, disorder and/or abnormality associated with TDP-43, or TDP-43 proteinopathy, is amyotrophic lateral sclerosis (ALS).

40. The TDP-43 binding molecule or immunoconjugate or labelled binding molecule or pharmaceutical composition for use according to claim 38, wherein the disease, disorder and/or abnormality associated with TDP-43, or TDP-43 proteinopathy, is Alzheimer's disease (AD).

41. The TDP-43 binding molecule or immunoconjugate or labelled binding molecule or pharmaceutical composition for use according to claim 38, wherein the disease, disorder, and/or abnormality associated with TDP-43, or TDP-43 proteinopathy, is Frontotemporal dementia (FTD).

42. A method of retaining or increasing cognitive memory capacity or slowing memory loss in an individual with a disease, disorder and/or abnormality associated with TDP-43 or a TDP-43 proteinopathy, comprising administering the TDP-43 binding molecule of any one of claims 1 to 21 or an immunoconjugate as claimed in any one of claims 22 to 28 or pharmaceutical composition of claim 30 to the individual.

43. A method of reducing the level of aggregated TDP-43 and/or phosphorylated TDP-43 in an individual, comprising administering the TDP-43 binding molecule of claims 1 to 21 or an immunoconjugate as claimed in any one of claims 22 to 28 or pharmaceutical composition of claim 30 to the individual.

44. The method of claim 42 or 43, wherein the method comprises administering at least one additional therapeutic agent.

45. The method of claim 44, wherein the additional therapeutic agent targets alpha-synuclein, BACE1, Tau, beta-amyloid, TDP-43 or a neuroinflammation protein.

46. A nucleic acid molecule encoding the TDP-43 binding molecule of any one of claims 1 to 21.

47. The nucleic acid molecule of claim 46, comprising a nucleotide sequence of SEQ ID NO: 18, SEQ ID NO: 19, SEQ ID NO: 28, SEQ ID NO: 29, SEQ ID NO: 38, SEQ ID NO: 39, SEQ ID NO: 48, SEQ ID NO: 49, SEQ ID NO: 58 or SEQ ID NO: 59.

48. A nucleic acid molecule encoding a TDP-43 binding molecule comprising the nucleotide sequences set forth as:
a. a Heavy Chain Variable Region (VH) encoded by SEQ ID NO: 58 and a Light Chain Variable Region (VL) encoded by SEQ ID NO: 59; or
b. a Heavy Chain Variable Region (VH) encoded by SEQ ID NO: 48 and a Light Chain Variable Region (VL) encoded by SEQ ID NO: 49; or
c. a Heavy Chain Variable Region (VH) encoded by SEQ ID NO: 38 and a Light Chain Variable Region (VL) encoded by SEQ ID NO: 39; or
d. a Heavy Chain Variable Region (VH) encoded by SEQ ID NO: 28 and a Light Chain Variable Region (VL) encoded by SEQ ID NO: 29; or
e. a Heavy Chain Variable Region (VH) encoded by SEQ ID NO: 18 and a Light Chain Variable Region (VL) encoded by SEQ ID NO: 19.

49. The nucleic acid molecule of claim 46, 47 or 48, wherein the nucleic acid is a part of a viral vector for targeted delivery to the blood brain barrier or any other cell type in the CNS.

50. The nucleic acid molecule of claim 49, wherein the targeted delivery is to endothelial cells of the blood brain barrier, pericytes of the blood brain barrier or astrocytes, preferably endothelial cells of the blood brain barrier.

51. The nucleic acid according to claim 50, wherein the viral vector is a recombinant adeno-associated viral vector (rAAV), preferably a recombinant adeno-associated viral vector selected from AAV1 to AAV12.

52. A recombinant expression vector comprising the nucleic acid of any one of claims 46 to 51.

53. A host cell comprising the nucleic acid of any one of claims 46 to 51 and/or the vector of claim 52.

54. A cell-free expression system containing the recombinant expression vector of claim 52.

55. A method for producing a TDP-43 binding molecule, in particular an antibody or antigen-binding fragment thereof, comprising the steps of:
a. culturing the host cell of claim 53 or cell-free expression system of claim 54 under conditions suitable for producing the TDP-43 binding molecule, in particular the antibody or antigen-binding fragment thereof; and
b. isolating the TDP-43 binding molecule, in particular the antibody or antigen-binding fragment thereof.

56. A method of detecting and/or quantifying TDP-43 in a sample obtained from a subject, the method comprising contacting the sample with a TDP-43 binding molecule according to any one of claims 1 to 21 and comparing the TDP-43 levels in the sample to those in a control sample or samples.

57. Use of a TDP-43 binding molecule of any one of claims 1 to 21 in a pairing assay comprising the steps of:
a. Incubating a sample with a capture antibody and a detect antibody;
b. Incubating the mixture obtained in step a with a reagent suitable for detection by the detect antibody;
c. Measuring the signal emitted by the detect antibody;
wherein the capture antibody is selected from an antibody as defined in any one of claims 1 to 21.

58. The use of a TDP-43 binding molecule according to claim 57, wherein the detect antibody is selected from an antibody as defined in any one of claims 1 to 21.

59. The method of claim 56 or the use of a TDP-43 binding molecule according to claim 57 or 58, wherein the sample is human blood, cerebrospinal fluid (CSF), interstitial fluid (ISF) and/or urine, preferably CSF.

60. A kit for diagnosis of a disease, disorder and/or abnormality associated with TDP-43, or a TDP-43 proteinopathy, or for use according to any one of claims 31 to 41, or for use in a method of any one of claims 42 to 45 comprising a TDP-43 binding molecule according to any one of claims 1 to 21.
